(19) United States
(12) Patent Application Publication
(10) Pub. No.: US 2021/0323987 A1
(43) Pub. Date: Oct. 21, 2021

(54) Methods for the Synthesis of Heteroatom Containing Polycyclic Aromatic Hydrocarbons

(71) Applicant: The Regents of the University of California, Oakland, CA (US)

(72) Inventors: Neil K. Garg, Oakland, CA (US); Evan R. Darzi, Oakland, CA (US); Joyann S. Barber, Oakland, CA (US); Robert Susick, Oakland, CA (US); Jason Chari, Oakland, CA (US); Katie Spence, Oakland, CA (US)

(73) Assignee: The Regents of the University of California, Oakland, CA (US)

(21) Appl. No.: 17/269,224

(22) PCT Filed: Aug. 20, 2019
(86) PCT No.: PCT/US19/47344

§ 371 Date: Feb. 17, 2021

Related U.S. Application Data
(60) Provisional application No. 62/720,005, filed on Aug. 20, 2018.

(51) Int. Cl. C07F 15/00; C07D 273/04; C07D 333/38; C07D 413/04; C07D 491/052; C07D 217/04; C07D 471/04; C07D 409/04; C07D 417/14; C07D 209/58; C07D 209/90; C07D 471/14; B01J 31/22; B01J 31/24

(52) U.S. Cl. CPC C07F 15/0053; B01J 2231/4272; C07D 333/38; C07D 413/04; C07D 491/052; C07D 217/04; C07D 471/04; C07D 409/04; C07D 417/14; C07D 209/58; C07D 209/90; C07D 471/14; B01J 31/2291; B01J 31/2226; B01J 31/2419; B01J 2531/824; C07D 273/04

(57) ABSTRACT

Methods for the synthesis of polycyclic aromatic hydrocarbons and synthesis platforms for performing such syntheses are provided. Methods and platforms are provided that allow for the synthesis of aza-polycyclic aromatic hydrocarbons by an expedient ring assembly. Methods and platforms allow for a modular approach to synthesis that provide multiple new C—C bonds in sequential pericyclic reactions, thus giving access to compounds with multiple axes of substitution.

### REFERENCE TO RELATED APPLICATIONS

The current application claims priority to U.S. Provisional Application No. 62/720,005, filed Aug. 20, 2018, the disclosure of which is incorporated herein by reference.

### STATEMENT OF FEDERAL RIGHTS

This invention was made with government support under Grant Numbers GM122245 and GM008496, awarded by the National Institutes of Health. The government has certain rights in the invention.

### FIELD OF THE INVENTION

The current disclosure is directed to methods for the synthesis of polycyclic aromatic hydrocarbons and heteroatom-containing polycyclic aromatic hydrocarbons, and more particularly to methods for the modular synthesis thereof by an expedient ring assembly, and to the heteroatom-containing polycyclic aromatic hydrocarbon products thereof.

### BACKGROUND OF THE INVENTION

Polycyclic aromatic hydrocarbons (PAHs) are important molecules in the fields of materials science and molecular electronics (see, for example, M. J. Allen, et al., Chem. Rev. 2010, 110, 132-145; and A. C. Grimsdale, et al., Chem. Commun. 2005, 2197-2204, the disclosure of which are incorporated herein by reference). Specifically, PAHs have been employed in many widely-used devices, such as organic light emitting diodes (OLEDs), field effect transistors (OFETs), and photovoltaics (OPVs) (for example, P. M. Beaujuge, et al., J. Am. Chem. Soc. 2011, 133, 20009-20029, the disclosure of which is incorporated herein by reference). However, synthetic methods to rapidly generate heterocyclic PAHs, and especially non-symmetric heteroatom-containing PAHs, remain limited. For example, the assembly of non-symmetric PAHs that possess multiple functional groups usually requires long synthetic sequences (M. Stepien, et al., Chem. Rev. 2017, 117, 3479-3716, the disclosure of which is incorporated herein by reference). In addition, synthetic approaches to PAHs often necessitate harsh reaction conditions (e.g., high temperatures and strongly acidic or basic conditions), and/or use of strongly basic organometallic reagents, or transition metal catalyzed cross-coupling reactions, and, still, typically result in low overall yields (T. C. McMahon, et al., Am. Chem. Soc. 2015, 137, 4082-4085; J. T. Markiewicz, F. Wudl, ACS Appl. Mater. Interfaces 2015, 7, 28063-28085; J. L. Marshall, et al., ChemPlusChem 2017, 82, 967-1001, the disclosure of which are incorporated herein by reference). Accordingly, new straightforward synthetic strategies allowing rapid access to a diverse range of unsymmetrical, heteroatom-containing PAHs are highly sought after.

### BRIEF SUMMARY OF THE INVENTION

The application is directed to methods for the synthesis of heteroatom-containing polycyclic aromatic hydrocarbons, and more particularly to methods for the modular synthesis of heteroatom-containing polycyclic aromatic hydrocarbons via in situ generated strained heterocylic alkynes or arynes, and to the heteroatom-containing polycyclic aromatic hydrocarbon products thereof.

Many embodiments are directed to methods for forming heteroatom-containing polycyclic aromatic hydrocarbons including:
providing a first cyclic alkyne, generated in situ from a first corresponding silyl triflate;
providing an oxadiazinone of formula:

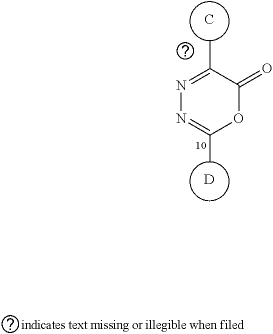

wherein rings C and D are functionalities individually chosen from: substituted or unsubstituted aromatic or heteroaromatic hydrocarbons, including polycyclic hydrocarbons;
providing a second cyclic alkyne or aryne, generated in situ from a second corresponding silyl triflate; and
reacting the first cyclic alkyne, the oxadiazinone, and the second cyclic alkyne or aryne in a plurality of sequential Diels-Alder reactions under reaction conditions to produce a polycyclic aromatic hydrocarbon comprising a 9,10-diarylanthracene scaffold.

In still many embodiments, the plurality of sequential Diels-Alder reactions includes a first Diels-Alder reaction, between the first cyclic alkyne and the oxadiazinone, to yield an intermediate pyrone; and a second Diels-Alder reaction, between the intermediate pyrone and the second cyclic alkyne or aryne, to yield the polycyclic aromatic hydrocarbon comprising a 9,10-diarylanthracene scaffold.

In yet many embodiments, the first cyclic alkyne includes in its ring at least one substituted or unsubstituted heteroatom selected from: N, O, S, Se, Si, B, P; and further comprises any number of substitutions and functional groups, each individually selected from: H, halide, alkyl, aryl, heteroaryl, alkoxy, PEG.

In still yet many embodiments, the first cyclic alkyne is 3,4,-piperidyne comprising an N-substitution selected from: H, alkyl, including Me, aryl, including phenyl, benzyl, carbamates, including Cbz and Boc, N-oxide, N-Borane.

In yet still many embodiments, the rings C and D, independently, include one or more functionality selected from: an electron-donating functional group, including para-methoxyphenyl, an electron-withdrawing functional group, including para-NO₂, and a halogen atom, including F, CI, Br, and I, heterocycles, including thiophene, alkenes, alkynes.

In still yet many embodiments, one or both of the rings C and D include a functional handle and wherein the functional handle is used to further extend, including polymerize, the polycyclic aromatic hydrocarbon comprising a 9,10-diarylanthracene scaffold and at least one heteroatom.

In yet still many embodiments, the second cyclic alkyne or aryne includes at least one feature selected from:
comprises at least one substituted or unsubstituted heteroatom selected from: N, O, S, Se, Si, B, P;
is polycyclic or polyheterocyclic, wherein the cycles are aromatic, non-aromatic, or both;
comprises any number of substitutions or functional groups, each individually selected from: H, alkyl, aryl, heteroaryl, electron-withdrawing groups, electron-donating groups.

In still yet many embodiments, the second cyclic alkyne or aryne is selected from: benzyne, naphthalyne, indolyne, and cyclohexyne, including cyclohexyne with at least one heteroatom, wherein the at least one heteroatom may be further functionalized.

In yet still many embodiments, the reaction conditions include additional reagents, reagent stoichiometry, and physical conditions selected to promote an elimination of silyl triflate from the first and the second corresponding silyl triflates, and to promote Diels-Alder reactions between the first cyclic alkyne and the oxadiazinone, and between an intermediate pyrone and the second cyclic alkyne or aryne.

In still yet many embodiments, the reaction conditions comprise an additional reagent providing F⁻, a solvent, a temperature, and a period of time.

In yet still many embodiments, the additional reagent providing F⁻ is selected from: CsF, LiF, KF, NaF, N(nBu)₄F, HF, HF.pyridine, Poly[4-vinylpyridinium poly(hydrogen fluoride)], tetrabutylammonium difluorotriphenylsilicate.

In still yet many embodiments, the solvent is selected from: acetonitrile, toluene, tetrahydrofuran, chloroform, dichloromethane, any other ethereal and halogenated solvents, and any mixture thereof.

In yet still many embodiments, the temperature is selected from: ambient, 30 to 60° C.

In still yet many embodiments, the period of time is 12 to 24 hours.

In yet still many embodiments, reacting the first cyclic alkyne, the oxadiazinone, and the second cyclic alkyne or aryne is conducted in a stepwise manner, wherein:
first, 1 equivalent of the first cyclic alkyne is reacted with 1 to 5 equivalents of the oxadiazinone and 1 to 10 equivalents of CsF in acetonitrile as 0.1 M solution relative to the first cyclic alkyne for 12 to 24 hours to produce an intermediate pyrone; and
next, 1 equivalent of the intermediate pyrone is reacted with 1 to 5 equivalents of the second cyclic alkyne or aryne and 1 to 10 equivalents of CsF in acetonitrile as 0.1 M solution relative to the intermediate pyrone for 12 to 24 hours.

In still yet many embodiments, wherein:
first, 1 equivalent of the first cyclic alkyne is reacted with 2 equivalents of the oxadiazinone and 2 equivalents of CsF in acetonitrile as 0.1 M solution relative to the first cyclic alkyne for 14 to 18 hours to produce an intermediate pyrone; and
next, 1 equivalent of the intermediate pyrone is reacted with 2 equivalents of the second cyclic alkyne or aryne and 5 equivalents of CsF in acetonitrile as 0.1 M solution relative to the intermediate pyrone for 18 hours.

In yet still many embodiments, the intermediate pyrone is isolated and purified prior to being reacted with the second cyclic alkyne or aryne.

In still yet many embodiments, the reacting of 1 equivalent of the first cyclic alkyne, 1 to 5 equivalents of the oxadiazinone, and 1 to 5 equivalents of the second cyclic alkyne or aryne is conducted in a one-pot manner, with addition of 1 to 10 equivalents of CsF in acetonitrile as 0.1 M solution relative to the first cyclic or heterocyclic alkyne for 12 to 24 hours.

In yet still many embodiments, the reacting of 1 equivalent of the first cyclic alkyne, 1 equivalent of the oxadiazinone, and 1 equivalent of the second cyclic alkyne or aryne is conducted in a one-pot manner, with addition of 3 equivalents of CsF in acetonitrile as 0.1 M solution relative to the first cyclic alkyne for 14 hours.

In still yet many embodiments, the polycyclic aromatic hydrocarbon comprising a 9,10-diarylanthracene scaffold further includes at least one heteroatom.

In yet still many embodiments, at least one heteroatom is nitrogen.

Various embodiments are directed to heteroatom-containing polycyclic aromatic hydrocarbon selected from the group consisting of:

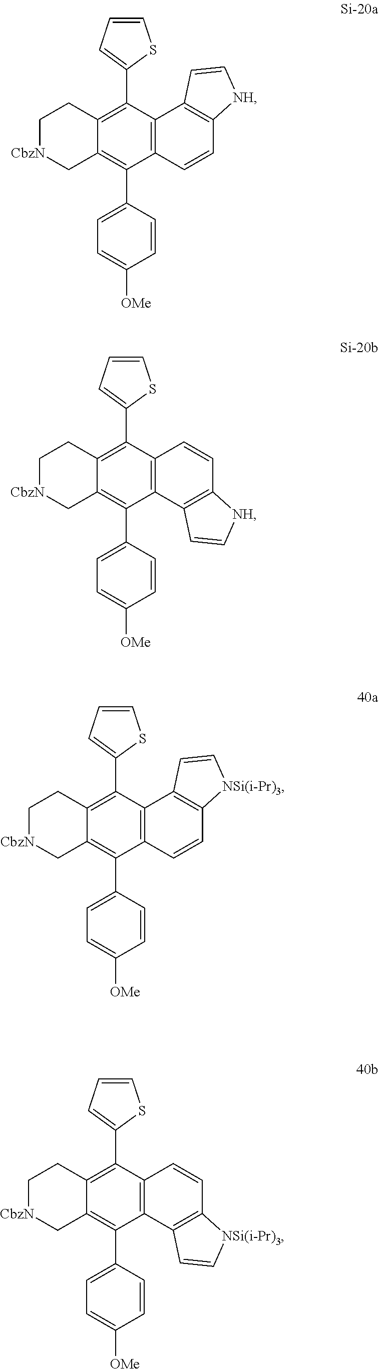

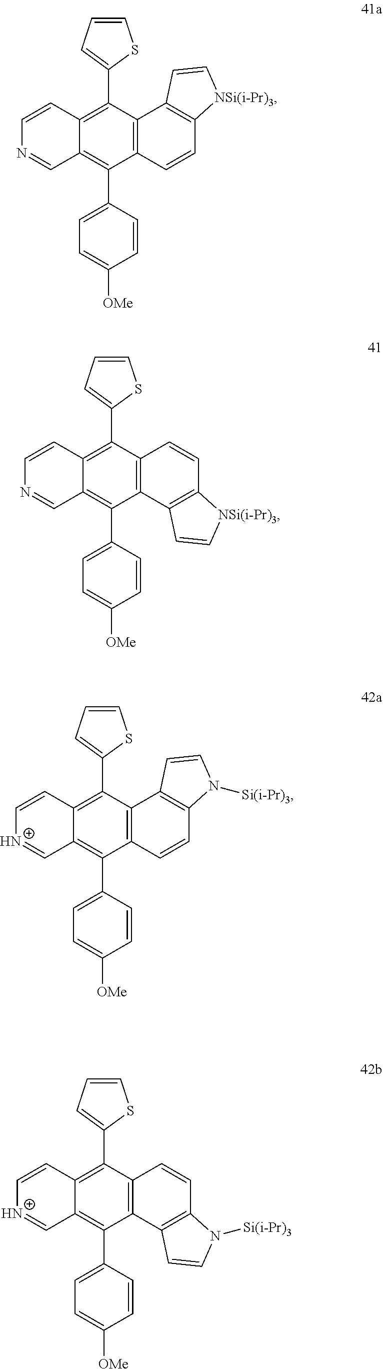

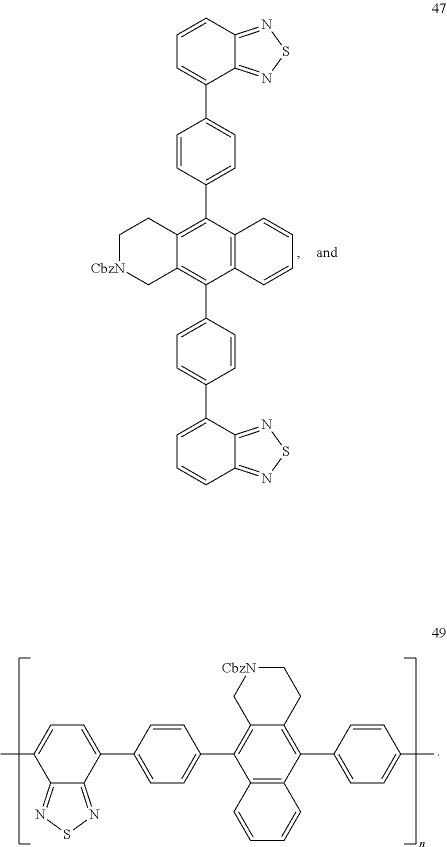

Some embodiments are directed to a method for forming polycyclic aromatic hydrocarbons including:
providing a cyclic alkyne or heterocyclic aryne, generated in situ from a corresponding silyl triflate;
providing a halo-biaryl; and
reacting the cyclic alkyne or aryne and the halo-biaryl in a transition metal-catalyzed cross-coupling reaction under reaction conditions to produce a polycyclic aromatic hydrocarbon comprising a triphenylene scaffold.

In still some embodiments, the cyclic alkyne or aryne comprises at least one feature selected from:
comprises at least one substituted or unsubstituted heteroatom selected from: N, O, S, Se, Si, B, P;
is polycyclic or polyheterocyclic, wherein the cycles are aromatic, non-aromatic, or both;
comprises any number of substitutions or functional groups, each individually selected from: H, alkyl, aryl, heteroaryl, electron-withdrawing groups, electron-donating groups.

In yet some embodiments, the cyclic alkyne or aryne is selected from: naphthalyne, indolyne, carbazolyne, and cyclohexyne, including cyclohexyne with at least one heteroatom, wherein the at least one heteroatom may be further functionalized.

In still yet some embodiments, the halo-biaryl is of formula:

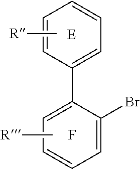

wherein rings E and F are functionalities individually chosen from: substituted or unsubstituted aromatic or heteroaromatic hydrocarbons, including polycyclic hydrocarbons; and
wherein R″ and R″′ are further functionalities individually chosen from H, alkyl, alkoxy, NO₂, amine, alkyl amine.

In yet still some embodiments, the halo-biaryl is selected from:

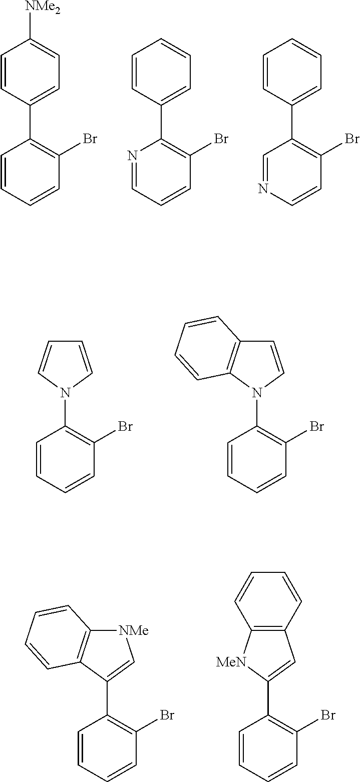

In still yet some embodiments, the reaction conditions include additional reagents, reagent stoichiometry, and physical conditions selected to promote an elimination of silyl triflate from the corresponding silyl triflate, and to promote the transition metal-catalyzed cross-coupling reaction between the cyclic alkyne or aryne and the halo-biaryl.

In yet still some embodiments, the reaction conditions include an additional reagent providing F⁻, the group 10 metal catalyst, a ligand, a solvent, reflux conditions, and a period of time.

In still yet some embodiments, the additional reagent providing F⁻ is selected from: CsF, LiF, KF, NaF, N(nBu)₄F, HF, HF.pyridine, Poly[4-vinylpyridinium poly(hydrogen fluoride)], tetrabutylammonium difluorotriphenylsilicate.

In yet still some embodiments, the cyclic alkyne or aryne is a cyclic or heterocyclic alkyne and an amount of CsOPiv is added.

In still yet some embodiments, the group 10 metal catalyst is Pd⁰ selected from: Pd(dba)₂ and Pd(OAc)₂; and the ligand including P(o-tolyl)₃.

In yet still some embodiments, the reflux conditions include a single or a mixture of solvents that can be heated to 90-150° C.

In still yet some embodiments, the period of time is 0.5 to 24 hours.

In yet still some embodiments, the reaction conditions comprise: 1 equivalent of the halo-biaryl, 2 equivalents of the cyclic alkyne or aryne, 1 to 20 equivalents of CsF, 5 to 100 mol % Pd⁰, 1:1 ratio of Pd⁰ to its ligand, a solvent or solvent mixture allowing heating to 90-150° C., 0.5 to 24 hours.

In still yet some embodiments, the reaction conditions comprise: 1 equivalent of the halo-biaryl, 2 equivalents of the cyclic alkyne or aryne, 10 equivalents of CsF, 5 mol % Pd(dba)₂, 5 mol % P(o-tolyl)₃, 1:1 acetonitrile/toluene 0.075M relative to halo-biaryl solvent mixture, 110° C., 24 hours.

In yet still some embodiments, the halo-biaryl is a part of a transition metal organometallic complex and the reaction conditions comprise: 1 equivalent of the halo-biaryl, 2 equivalents of the cyclic alkyne or aryne, 10 equivalents of CsF, 10 mol % Pd(OAc)₂, 10 mol % P(o-tolyl)₃, 1:1 acetonitrile/toluene 0.075M relative to halo-biaryl solvent mixture, 110° C., 0.5 hours.

In yet still some embodiments, the transition metal organometallic complex comprises a transition metal selected from: Co, Ir, Rh, Ni, Pd, Pt, Zn, Cu, Fe, Mn, Os.

In still yet some embodiments, at least one heteroatom is employed to further decorate or otherwise extend the polycyclic aromatic hydrocarbon comprising a triphenylene scaffold and at least one heteroatom.

In still yet some embodiments, the halo-biaryl is bromo-biaryl.

Certain embodiments are directed to a A heteroatom-containing polycyclic aromatic hydrocarbon selected from the group consisting of:

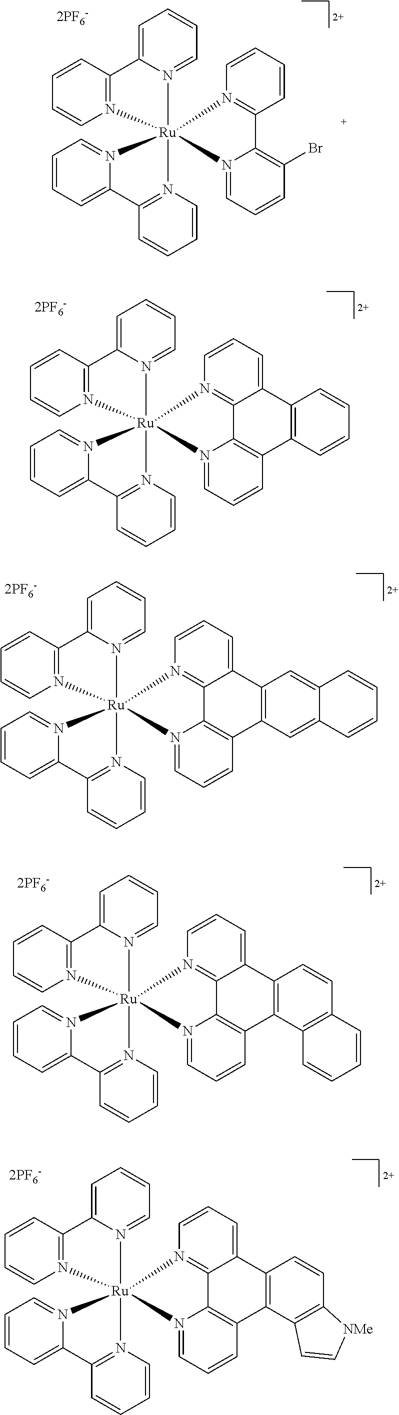

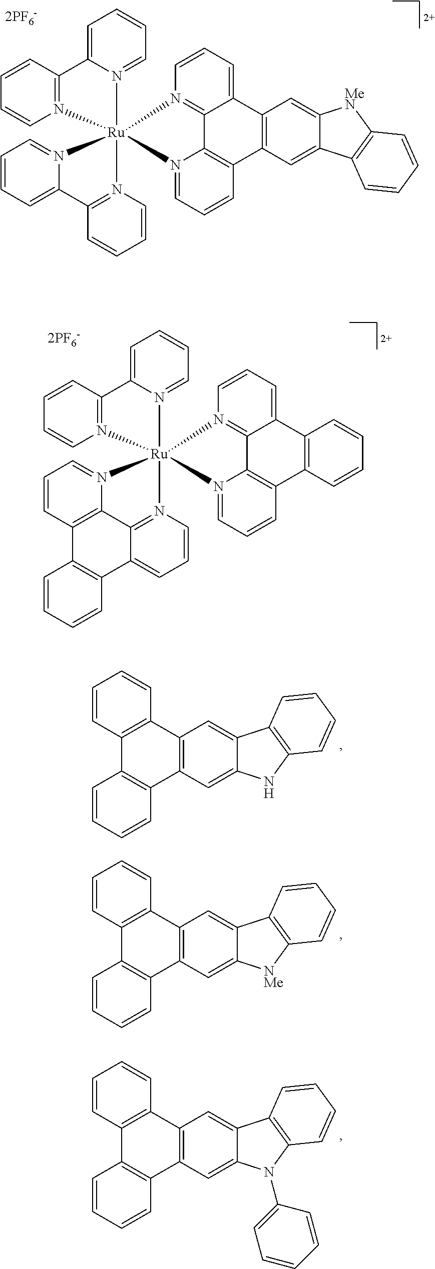

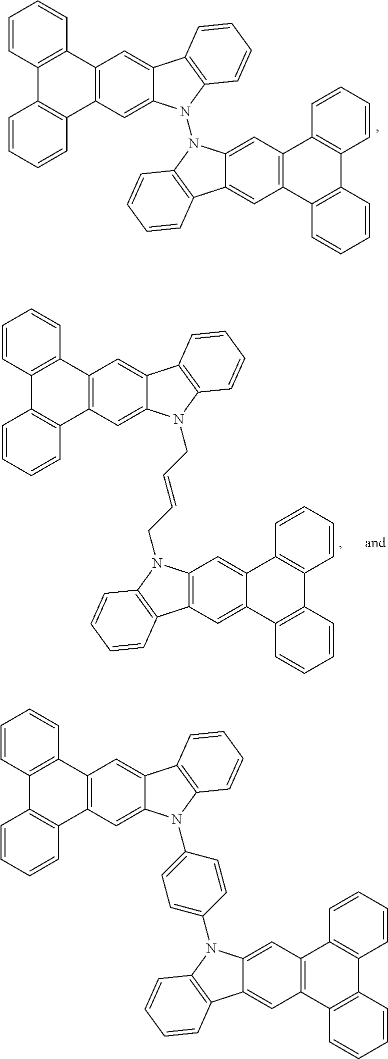 [SMILES: c1ccc2c(c1)c1ccccc1c1cc3c(cc21)c1ccccc1n3-n1c2ccccc2c2cc3c4ccccc4c4ccccc4c3cc21 ; C(=C\Cn1c2ccccc2c2cc3c4ccccc4c4ccccc4c3cc21)/Cn1c2ccccc2c2cc3c4ccccc4c4ccccc4c3cc21 ; c1ccc2c(c1)c1ccccc1c1cc3c(cc21)c1ccccc1n3-c1ccc(-n2c3ccccc3c3cc4c5ccccc5c5ccccc5c4cc32)cc1]

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIG. 1A provides examples of desirable polycyclic aromatic hydrocarbons (PAHs), in accordance with prior art.

FIG. 1B provides examples of cyclic alkynes, arynes, and heteroatom-containing variants thereof, in accordance with prior art.

FIG. 2A provides a schematic for 9,10-diphenylanthracene-based PAH scaffold with four quadrants of differentiation, while FIG. 2B provides a reaction diagram for forming the same, and FIG. 2C provides some examples of reaction components and possible products, in accordance with embodiments of the invention.

FIG. 3 provides a reaction diagram, with a chemically specific example, for installation of one of the quadrants of differentiation in the formation of heteroatom-containing PAHs and, more specifically, for forming a pre-PAH intermediate pyrone, together with a table illustrating the importance of reaction stoichiometry, in accordance with embodiments of the invention.

FIG. 4A provides a reaction diagram for installation of the second quadrant of differentiation in formation of heteroatom-containing PAHs, while FIG. 4B provides a table illustrating the diversity of resulting PAHs in accordance with embodiments of the invention.

FIG. 5 provides a reaction diagram for additional diversification of heteroatom-containing PAHs and examples of PAHs with four chemically different quadrants, in accordance with embodiments of the invention.

FIG. 6 provides an example of a three component/one pot process to produce a heteroatom-containing PAH in accordance with embodiments of the invention.

FIG. 7A provides a schematic for triphenylene-based PAH scaffold with three locations for differentiation, while FIG. 7B provides a reaction diagram for forming the same, in accordance with embodiments of the invention.

FIGS. 8A and 8B provide illustrative examples of the scope and diversity of the silyl triflate candidates for use in the formation of triphenylene scaffold PAHs, in accordance with embodiments of the invention.

FIG. 9 provides illustrative examples of the scope and diversity of the bromo-biaryl candidates for use in the formation of triphenylene scaffold PAHs, in accordance with embodiments of the invention.

FIG. 10 illustrates rapid diversification of carbazole scaffold PAH in accordance with embodiments of the invention.

FIGS. 11A and 11B illustrate strategic synthesis of a complex pH-responsive fluorophore in accordance with embodiments of the invention.

FIGS. 12A and 12B provide reaction diagrams for rapidly forming exemplary monomeric and polymeric fluorophores, while FIGS. 12C-12H provide spectroscopic data, including absorbance and emission spectra, for the same, in accordance with embodiments of the invention.

FIGS. 13A and 13B provide examples of diversification of useful Ru organometallic complexes in accordance with embodiments of the invention.

### DETAILED DESCRIPTION OF THE INVENTION

Turning now to the drawings, methods for the syntheses of polycyclic aromatic hydrocarbons (PAHs) and heteroatom-containing PAHs and platforms for performing such syntheses are provided. In many embodiments methods and platforms are provided that allow for the synthesis of aza-polycyclic aromatic hydrocarbons by an expedient ring assembly. Various such embodiments employ modular approaches that rely on the controlled generation of transient cyclic and heterocyclic alkynes, arynes and heteroarynes to provide multiple new C—C bonds in predetermined sequential reactions, thus giving access to diverse compounds with multiple axes of substitution. In some embodiments, four new C—C bonds are formed via sequential pericyclic reactions. In other embodiments, two new C—C bonds are formed via transition metal catalyzed couplings. In many embodiments, the synthetic sequences disclosed in the instant application are performed in a stepwise fashion, while in other embodiments, the same is achieved in a one-pot fashion. In many embodiments, previously inaccessible PAHs and new valuable organic materials are produced.

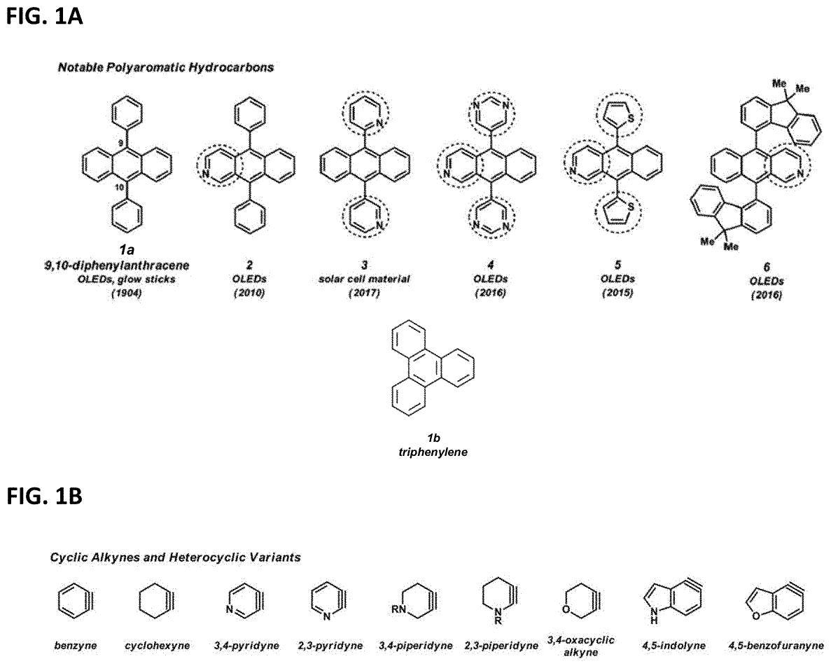

An important subset of PAHs are 9,10-diphenylanthracene derivatives. The parent compound, 9,10-diphenylanthracene (1a in FIG. 1A) has been widely studied since 1904 (A. Haller, et al., Seances Acad. Sci. 1904, 138, 1251-1254, the disclosure of which is incorporated herein by reference) and has been used in blue glow sticks (J. H. Carmel, et al., J. Chem. Educ. 2017, 94, 626-631, the disclosure of which is incorporated herein by reference) and OLEDs (W. J. Jo, et al., Synth. Met. 2009, 159, 1359-1364. the disclosure of which is incorporated herein by reference). Novel derivatives of 1a have been highly sought (M. Chen, et al., J. Mater. Chem. C 2018, 6, 7416-7444, the disclosure of which is incorporated herein by reference). In particular, adding heteroatoms into the 1a scaffold allows to modulate the properties and potential applications of such PAHs (J. E. Anthony, Chem. Rev. 2006, 106, 5028-5048, the disclosure of which is incorporated herein by reference). Heteroatoms may be included in the anthracene ring itself or on the C9/C10 substituents, as exemplified by 2 (see, F. Eiden, et al., Arch. Pharm. 1986, 319, 886-889; C. Bozzo, et al., Heterocycl. Commun. 1996, 2, 163-168; J. Li, et al., Dyes Pigm. 2015, 112, 93-98; S. J. Eum, et al., U.S. Pat. No. 8,153,279, 2012, the disclosure of which are incorporated herein by reference) and 3 (X. Li, et al., Angew. Chem. Int. Ed. 2017, 56, 5598-5602; Angew. Chem. 2017, 129, 5690-5694, the disclosure of which are incorporated herein by reference) respectively (FIG. 1A), and can impact material properties. Compounds possessing heteroatoms on both the anthracene ring and C9/C10 substituents, such as 4 of FIG. 1A, have also been prepared in the context of OLEDs (C. Gao, et al., Chinese Patent CN10508599, 2018, the disclosure of which is incorporated herein by reference). Furthermore, more exotic analogs of 1a and 2 have been prepared, where the C9/C10 substituents are replaced with heterocycles or substituted aromatics, as demonstrated by compounds 5 and 6 (C. Xia, et al., U.S. Patent US2016/0149139, 2016, the disclosure of which is incorporated herein by reference) (FIG. 1A). Another useful subset of PAHs are derivatives of triphenylene scaffolds (1b, FIG. 1A), which are also attractive molecules for optics and electronics applications, especially if such scaffolds can be diversified with addition of heteroatoms and other relevant functionalities. However, such additions typically require many additional synthetic steps and are generally difficult to achieve. All these PAH examples, many of which have been developed only recently, reflect a rapidly growing area of discovery (C. Gao, et al., World Patent WO2016/192346, 2016; and D.-H. Kim, et al., Korean Patent KR10-2010-0108120, 2010, the disclosure of which are incorporated herein by reference).

Some synthetic strategies towards PAHs rely on small, alkyne-containing aromatic rings-arynes (A. V. Dubrovskiy, et al., Org. Biomol. Chem. 2013, 11, 191-218; D. Perez, et al., Eur. J. Org. Chem. 2013, 5981-6013; X. Xiao, T. R. Hoye, Nat. Chem. 2018, 10, 838-844; S. E. Suh, et al., Chem. Sci. 2015, 6, 5128-5132; and Y. Mizukoshi, et al., J. Am. Chem. Soc. 2015, 137, 74-77, the disclosure of which are incorporated herein by reference). In recent years, strained cyclic alkynes, such as those depicted in FIG. 1B (A. E. Goetz, N. K. Garg, Nat. Chem. 2013, 5, 54-60; T. C. McMahon, et al., J. Am. Chem. Soc. 2015, 137, 4082-4085; C. Wentrup, et al., J. Am. Chem. Soc. 1988, 110, 1874-1880; S. F. Tlais, et al., J. Am. Chem. Soc. 2014, 136, 15489-15492; T. K. Shah, et al., J. Am. Chem. Soc. 2016, 138, 4948-4954; A. E. Goetz, et al., Chem. Commun. 2015, 51, 34-45; G.-Y. J. Im, et al., J. Am. Chem. Soc. 2010, 132, 17933-17944; M. G. Reinecke, Tetrahedron 1982, 38, 427-498, the disclosure of which are incorporated herein by reference) have gained significant attention and have been widely employed in synthetic methodology studies (R. Sanz, Org. Prep. Proced. Int. 2008, 40, 215-291; S. Yoshida, T. Hosoya, Chem. Lett. 2015, 44, 1450-1460; and S. S. Bhojgude, et al., Acc. Chem. Res. 2016, 49, 1658-1670, the disclosure of which are incorporated herein by reference). Such efforts have led to a greater understanding of aryne and cyclic alkyne reactivity and regioselectivities (V. Diemer, et al., Eur. J. Org. Chem. 2011, 341-354; P. H.-Y. Cheong, et al., J. Am. Chem. Soc. 2010, 132, 1267-1269; and N. F. Fine Nathel, et al., J. Am. Chem. Soc. 2016, 138, 10402-10405, the disclosure of which are incorporated herein by reference), and a host of synthetic applications impacting catalysis (C. C. Mauger, G. A. Mignani, Org. Process Res. Dev. 2004, 8, 1065-1071, the disclosure of which is incorporated herein by reference), agrochemistry (F. Schleth, et al., World Patent WO2011131544 A1, 2011, the disclosure of which is incorporated herein by reference), pharmaceuticals, academia (C. M. Gampe, E. M. Carreira, Angew. Chem. Int. Ed. 2012, 51, 3766-3778; Angew. Chem. 2012, 124, 3829-3842; and P. M. Tadross, B. M. Stoltz, Chem. Rev. 2012, 112, 3550-3577, the disclosure of which are incorporated herein by reference), and materials chemistry, including, more specifically, synthesis of PAHs.

This application is directed to embodiments of modular and rapid methods for syntheses of a diverse range of PAHs, including heteroatom-containing PAHs and PAHs that comprise both aromatic and non-aromatic rings, and further including small molecule fluorophores and conductive polymers. More specifically, in many embodiments, the synthetic methods of the instant application rely on trapping of in situ generated strained intermediates, that are transient cyclic and heterocyclic alkynes, arynes and heteroarynes, to furnish a multitude of diverse PAH scaffolds, including unsymmetrical, heteroatom-containing, and otherwise highly complex structures. In many embodiments, the PAH scaffolds resulting from the methods of the instant application comprise a plurality of aromatic and non-aromatic rings. In many embodiments, the transient intermediates are cyclic and heterocyclic hexynes. In many embodiments, the methods of the application allow for facile installation of heteroatoms and other desired molecular segments or functionalities into the PAH scaffold via judicious selection of simple reactive components. In some embodiments the full synthetic sequence is conducted stepwise, while in other embodiments, the desired PAHs are produced in a one-pot manner.

### 9,10-Diphenylanthracene Scaffold PAHs

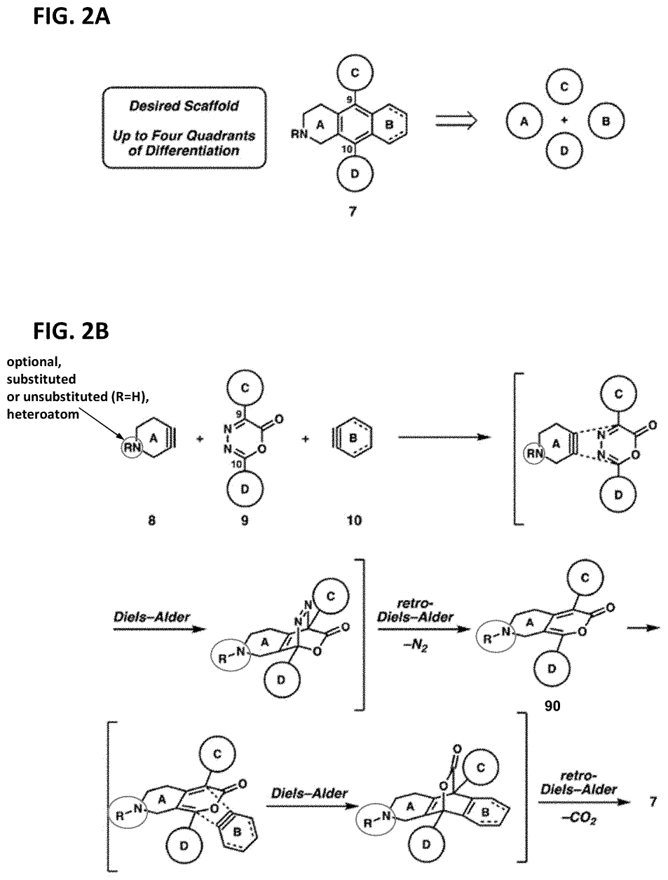

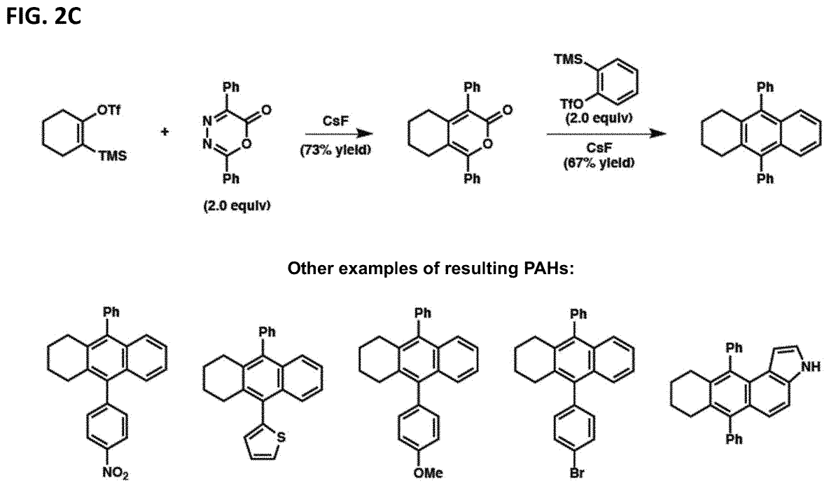

FIG. 2A depicts a 9,10-diphenylanthracene-based scaffold (7) targeted by the synthetic methods of some embodiments of the instant application. In many such embodiments, ring fragments A-D of scaffold 7 are united with formation of the central benzene ring (FIG. 2A). More specifically, in many embodiments, formation of scaffold 7 via synthetic methods of the instant application enables access to a diverse range of PAH scaffolds, with the possibility of accessing four quadrants (rings A, B, C, and D in FIG. 2A) of differentiation. To this end, FIG. 2B provides the schematically depicted process for the formation of such scaffold 7, wherein, according to many embodiments, reactive cyclohexynes (compounds 8) and cyclohexynes or arynes (compounds 10) are used as building blocks A and B of the PAH scaffold, respectively. Accordingly, in many embodiments, the strategic use of heteroatom-containing cyclic strained intermediates 8 and 10 allows to access the desired heteroatom-containing PAHs (for examples of potentially desirable heteroatom-containing PAHs, see, for example J. B. Lin, et al., J. Am. Chem. Soc. 2017, 139, 10447-10455, the disclosure of which is incorporated herein by reference), wherein the heteroatom is situated on the anthracene ring. For example, the use of 3,4-piperidyne as 8 allows straightforward access to otherwise difficult to achieve isoquinoline scaffolds.

Furthermore, in many embodiments, and further according to FIG. 2B, building blocks C and D of scaffold 7 are introduced via a readily accessible oxadiazinone core 9. In many such embodiments, oxadiazinones (or diazapyrones), such as 9, are easily prepared from simple precursors (see, for example, M. L. Tintas, et al., J. Mol. Struct. 2014, 1058, 106-113, the disclosure of which is incorporated herein by reference) and are known to readily undergo one or more Diels-Alder (DA) cycloaddition/retro-Diels-Alder (rDA) cycloaddition reactions (with sequential expulsion of N₂ and CO₂) (B. Rickborn, Org. React. 1998, 53, 223-629, the disclosure of which is incorporated herein by reference). Therefore, in many embodiments, heteroatoms or other desired functionalities are introduced into the PAHs of the instant application as part of C9/C10 substituents via judicious choice of blocks C and D.

More specifically, the synthetic methods of the instant application provide a means to allow for the controlled generation and trapping of fragments 8 and 10 to ultimately deliver scaffolds 7 through the cascade of events suggested in FIG. 2B. Specifically, in many embodiments, the synthesis of 7, proceeds as generally depicted in FIG. 2B, wherein a first strained cycloalkyne or heterocycloalkyne 8, first reacts with the provided oxadiazinone 9 in a Diels-Alder reaction, followed by the resulting intermediate undergoing a retro Diels-Alder with expulsion of nitrogen gas to produce a pyrone intermediate 90. In turn, in many embodiments, pyrone 90 next reacts with a second cycloalkyne/heterocycloalkyne or aryne/heteroaryne 10 in another DA, followed by another rDA, this time with expulsion of CO₂, to yield the desired PAH 7. In some embodiments, the synthetic sequence for obtaining 7 is performed stepwise, with isolation and purification of intermediates. In other embodiments, the synthetic sequence for obtaining 7 is performed in a one-pot fashion.

Here, it should be noted, that Steglich (W. Steglich, et al., Synthesis 1977, 252-253, the disclosure of which is incorporated herein by reference) had previously demonstrated the double addition of benzyne into oxadiazinones, in addition to the syntheses of conjugated materials by Nuckolls (Q. Miao, et al., J. Am. Chem. Soc. 2006, 128, 1340-1345, the disclosure of which is incorporated herein by reference) and Wudl (D. Chun, et al., Angew. Chem. Int. Ed. 2008, 47, 8380-8385; Angew. Chem. 2008, 120, 8508-8513, the disclosure of which is incorporated herein by reference) employing the same. However, an important limitation in all these cases is the inability to incorporate two different strained alkynes, instead delivering symmetric products with respect to building blocks A and B. It should also be noted, that DA reactions using substituted tetrazenes or isobenzofurans are also known, and also suffer from the inability to introduce two different strained intermediates in a controlled fashion. (For examples, see: S.-E. Suh, et al., Chem. Sci. 2018, 9, 7688-7693, the disclosure of which are incorporated herein by reference.) In contrast, the synthetic methods of some embodiments of the instant application allow a facile diversification of all four quadrants of the 9,10-diphenylanthracene scaffolds, as depicted, for example, in FIG. 2C.

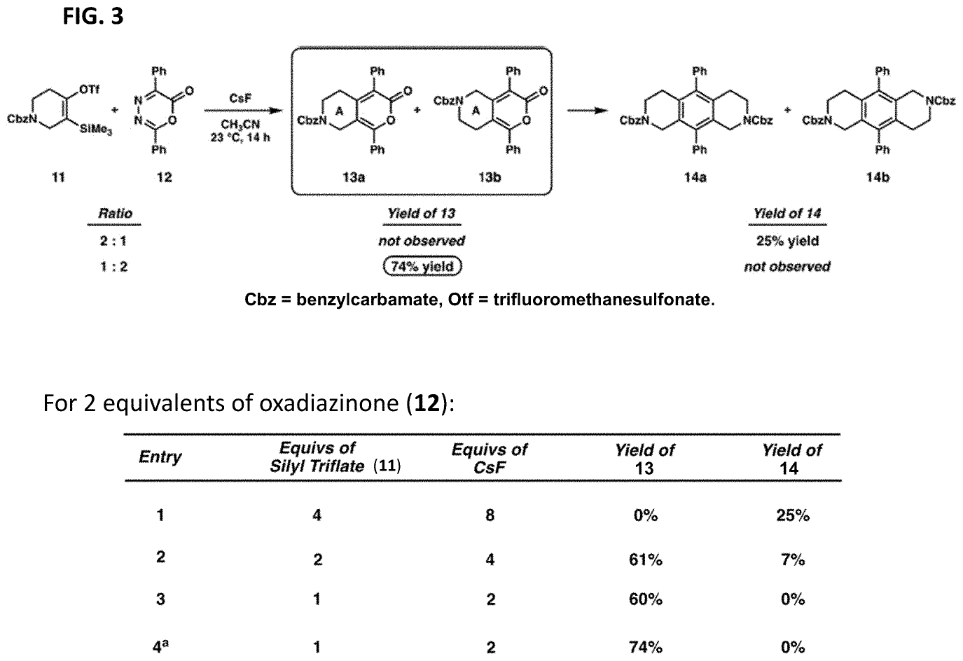

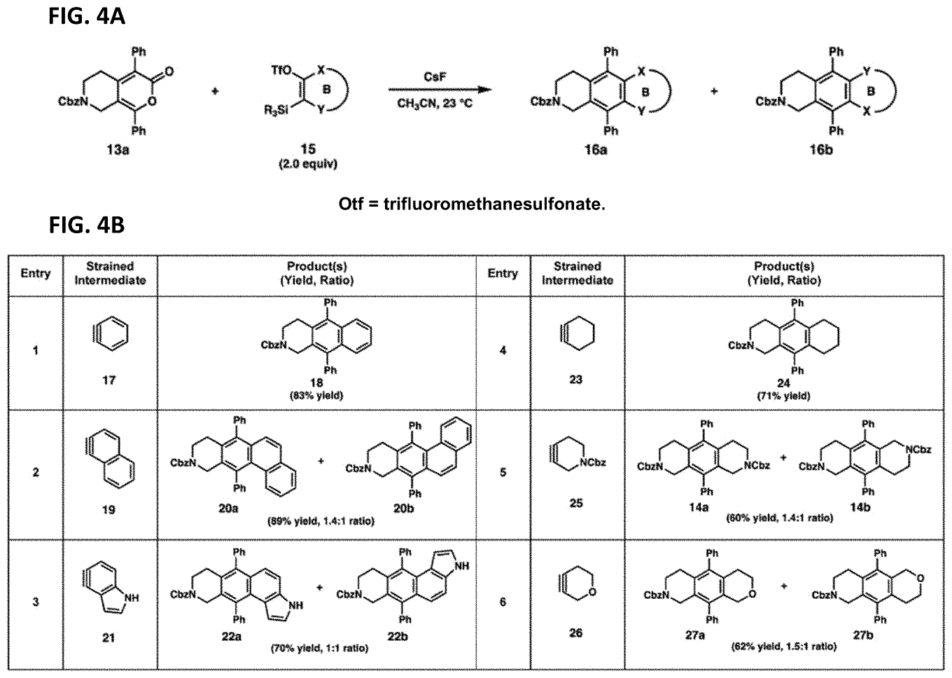

In order to further demonstrate and explain some of the principles of the methods of the instant application, FIGS. 3 and 4 provide illustrative and more detailed (than FIG. 2B) examples of the chemistries and processes employed in the production of scaffolds 7 at different stages of the overall reaction sequence. Accordingly, FIG. 3 demonstrates the oxadiazinone trapping with the first strained intermediate generated in situ to produce pyrone 13, and FIG. 4 demonstrates the conversion of 13 to the desired scaffold 7. In these examples, 13 is the simplest version of pyrone 90, wherein fragments C and D are both phenyl rings, however, any other oxadiazinone variant can be used according to many other embodiments of the instant application. Furthermore, although FIGS. 3 and 4 illustrate the methods of the instant application in a step-wise fashion, in many embodiments, scaffold 7 PAHs can be obtained in a one-pot procedure, without isolation of intermediates, such as pyrone intermediates.

As mentioned above, arynes, such as benzyne, are known to undergo oxadiazinone trapping. However, the resulting intermediate benzopyrone directly undergoes trapping with a second equivalent of the same aryne, precluding the opportunity to add two different strained alkyne fragments into the desired scaffold. Therefore, reacting benzyne with a simple model oxadiazinone 12 (wherein 12 is oxadiazinone 9 with phenyls for blocks C and D) produces only a double benzyne addition product—symmetric 9,10-diphenylanthracene 1a. Accordingly, although not to be bound by any theory, it is hypothesized, that the intermediate benzopyrone is more reactive than the initial oxadiazinone, and, therefore, prevents isolation or second addition of a different aryne. Nevertheless, Sauer and co-workers were able to trap non-aromatic cyclooctyne with an oxadiazinone to produce a pyrone intermediate (J. Balcar, et al., Tetrahedron Lett. 1983, 24, 1481-1484 the disclosure of which is incorporated herein by reference). However, no further reactions of this pyrone were described. Also of note, the cyclooctyne used by Sauer is stable and does not have to be generated in situ and furthermore cannot subsequently be transformed to an aromatic PAH. Accordingly, the methods of the instant application employ cyclic alkynes and optimized stoichiometry to trap the intermediate pyrone 90, yet avoid the second addition of the same alkyne. In many preferred embodiments, and in contrast to Sauer, the cyclic alkynes comprise 6-membered rings and are cyclohexynes or heterocyclohexynes. In many such embodiments, 6-membered ring alkynes possess a host of advantageous properties, including very good stability, which allows them to be isolated and makes them easier to use. In addition, in many embodiments, 6-membered ring alkynes yield PAHs with advantageous electronic and materials properties. However, in many other embodiments, the methods of the application may also rely on trapping of fleeting intermediates, which cannot be isolated, including cycloalkynes and heterocycloalkynes of various other ring sizes.

In many embodiments, cycloalkynes 8 further optionally comprise any number of desired functional groups and substitutions and any number of ring heteroatoms selected from: N, P, O, S, Se, B, Si; which, in turn, may be further substituted. In many embodiments 8 comprises at least one nitrogen atom. In many embodiments, 8 further comprises any number of substitutions and functional groups, including groups selected from: H, halide, alkyl, aryl, heteroaryl, alkoxy, PEG. In many embodiments, 8 is 3,4-piperidyne. In many such or other embodiments, 8 also comprises an N-substitution selected from: H, alkyl, including Me, aryl, including phenyl, benzyl, carbamates, including Cbz and Boc, N-oxide, N-Borane. However, in some embodiments 8 is a simple cyclohexyne (FIG. 2C). In many embodiments, the cyclic and heterocyclic alkynes are generated in situ from the corresponding silyl triflates via Kobayashi elimination (Y. Himeshima, T. Sonoda, H. Kobayashi, Chem. Lett. 1983, 12, 1211-1214, the disclosure of which is incorporated herein by reference). In turn, in many such embodiments, the desired silyl triflates can be purchased or prepared according to known methodologies. For example, silyl triflate 11 of FIG. 3 is commercially available, but can also be prepared as needed in 3 steps from 4-methoxypyridine).

FIG. 3 illustrates the installation of the 1^{st} quadrant A of the PAH scaffold 7, wherein Cbz protected 3,4-piperidyne (i.e., 8 with R=Cbz) generated in situ from 11 is trapped by oxadiazinone 12 to yield intermediate pyrones 13a and 13b (i.e., in this example, the simplest, diphenyl version of 90), according to many embodiments. In addition, FIG. 3 illustrates the effect the stoichiometry of the reaction components has on the outcome of the synthetic methods of the instant application. Notably, when silyl triflate of 8 (or its variants such as 11 in FIG. 3) is used in excess relative to the provided oxadiazinone 9 (or its variants such as 12 in FIG. 3), i.e, the components are combined in a 2:1 ratio respectively, the major products obtained are double adducts, such as 14a and 14b, and no pyrone intermediate is observed (FIG. 3 and entry 1 of the Table in FIG. 3), consistent with the results previously seen in oxadiazinone reactions with arynes. However, when, in many embodiments, an inverted 1:2 ratio of silyl triflate (e.g., 11) to oxadiazinone (e.g., 12) is utilized, the desired pyrone intermediates (e.g., 13a and 13b), arising from a single DA/rDA reaction, are isolated in high yield, without formation of the double addition products (FIG. 3 and entries 3 and 4 of the Table in FIG. 3). Furthermore, altering the stoichiometry, in accordance with various other embodiments, to a 1:1 ratio, produces both the pyrones (13a and 13b) and the double addition products (14a and 14b) (entry 2 of the Table in FIG. 3). Accordingly, in many embodiments, silyl triflate of 8 is combined with 1 to 5 equivalents of oxadiazinone.

In addition, other reaction conditions of the instant methods, such as additional reagents, concentration, solvent choice, temperature, and duration can also be optimized according to many embodiments to improve the yield of the single-adduct intermediate pyrone and, therefore, of the desired PAH scaffolds. For example, by increasing the concentration of silyl triflate of 8 to 0.1 M, in accordance with various embodiments, it is possible to obtain pyrones (13a and 13b) in 74% yield (entry 4 of the Table in FIG. 3), which is advantageous in many embodiments of the methods and systems. Overall, in many embodiments, the reaction conditions comprise ambient temperature and acetonitrile solvent. However, in some embodiments, the reaction may be heated to 30-60° C., and the choice of solvent may be adjusted accordingly. In many embodiments a mixture of solvents is employed, such as acetonitrile/toluene mixture. In many such embodiments, heating may reduce the reaction time. However, in many embodiments, the reaction time is 12 to 24 hours. In some embodiments the solvent is selected from: acetonitrile, toluene, tetrahydrofuran, chloroform, dichloromethane, any other ethereal and halogenated solvents, and any mixture thereof.

Furthermore, it should be noted, that although CsF reagent is used in many examples of the instant application to promote Kobayashi elimination of silyl triflate, any reagent that promotes elimination of silyl triflate to produce alkyne may be used according to embodiments. For example, in some embodiments, any reagent selected from: CsF, LiF, KF, NaF, N(nBu)₄F, HF, HF.pyridine, Poly[4-vinylpyridinium poly(hydrogen fluoride)], tetrabutylammonium difluorotriphenylsilicate, is used to generate a cycloalkyne or heterocycloialkyne in situ from the corresponding alkene silyl triflate. However, as mentioned above, in some embodiments, pyrone intermediate is produced as a mixture of regioisomers, such as 13a and 13b in FIG. 3, and, in many embodiments, the treatment of such mixture with excess CsF under oxidative conditions selectively decomposes 13b leaving 13a untouched. Furthermore, in many embodiments, the silyl triflate elimination reagent is used in excess relative to the silyl triflate-bearing compound (e.g., 11). In many embodiments, silyl triflate is combined with 1 to 10 equivalents of CsF or another Kobayashi elimination promoting reagent.

In summary, FIG. 3 demonstrates several important aspects of the synthetic methods of the instant application for formation of diverse PAHs. First, in many embodiments and for the first time, a selected strained cyclic alkyne intermediate derived from a Kobayashi silyl triflate precursor participates in a single cycloaddition with a provided oxadiazinone when reacted in proper stoichiometry. In many embodiments, the proper stoichiometry comprises equal amount or excess of oxadiazinone reaction component. In addition, in many embodiments, the processes of the instant application occur under exceptionally mild reaction conditions, such as ambient temperature and few simple reagents. Furthermore, in some embodiments, the methods of the instant application produce mixtures of pyrone regioisomers (e.g., 13a and 13b), further diversifying the pool of potential PAHs. Nevertheless, in some such embodiments a single regioisomer can be isolated in good yield, as in the case of 13a, and used to obtain the desired PAHs.

Next, FIG. 4A illustrates the installation of 2^{nd} quadrant B of the PAH scaffold 7 by adding it to pyrone intermediate 90 (i.e., illustrative example 13a) according to many embodiments, while the table in FIG. 4B demonstrates the diversity of resulting PAHs. More specifically, in many embodiments, as illustrated in FIGS. 4A and 4B, pyrones of embodiments, such as 13a, readily undergo a DA/rDA reaction sequence, with loss of CO₂, in the presence of arynes or nonaromatic cyclic alkynes generated in situ from silyl triflate precursors 15. In many embodiments, this reaction proceeds at ambient temperature, with addition of excess of CsF (or another reagent promoting Kobayashi elimination), in CH₃CN (0.1M relative to pyrone), for 12 to 24 hours. Furthermore, in many embodiments, 1 equivalent of pyrone is reacted with 1 to 5 equivalents of 15, and 1 to 10 equivalents of CsF. In many embodiments, 1 equivalent of pyrone is reacted with 2 equivalents of 15, and 5 equivalents of CsF. In many embodiments, the transformation proceeds with formation of two new C—C bonds and delivers non-symmetric (if desired) heterocyclic PAH skeletons, such as, for example, 16. FIG. 4B provides several examples of strained intermediates that can be obtained from corresponding silyl triflates 15 and used in the formation of desired PAHs, according to many embodiments. In many embodiments, 15 comprises at least one feature selected from: comprises at least one substituted or unsubstituted heteroatom selected from: N, O, S, Se, Si, B, P; is polycyclic or polyheterocyclic, wherein the cycles are aromatic, non-aromatic, or both; comprises any number of substitutions or functional groups, each individually selected from: H, alkyl, aryl, heteroaryl, electron-withdrawing groups, electron-donating groups. More specifically, strained aryls and heteroaryls: benzyne 17, 1,2-naphthalyne 19 (D. Pena, et al., Org. Lett. 1999, 1, 1555-1557, the disclosure of which is incorporated herein by reference), and 4,5-indolyne 21—all perform well in the methods of the embodiments, giving rise to products 18, 20 and 22 in good yields (entries 1-3 in FIG. 4B). In addition, non-aromatic cyclic alkynes, such as cyclohexyne 23, and heterocyclic strained cyclic alkynes 25 and 26 also perform smoothly (entries 4-6 FIG. 4B). In many embodiments, non-aromatic cyclic alkynes offer greater than aryls' sp3-character and improved solubility of eventual PAH products. Notably silyl triflate precursors to 17, 19, 21, and 25 are all commercially available. With regard to regioselectivities (entries 2, 5, and 6 in FIG. 4B), although not to be bound by any theory, the major product likely arises from initial bond formation occurring between the more electron-rich carbon adjacent to the carbonyl group of the pyrone (K. Afarinkia, et al., Tetrahedron 1992, 48, 9111-9171, the disclosure of which is incorporated herein by reference) and the more distorted carbon of the strained intermediate in a concerted asynchronous fashion.

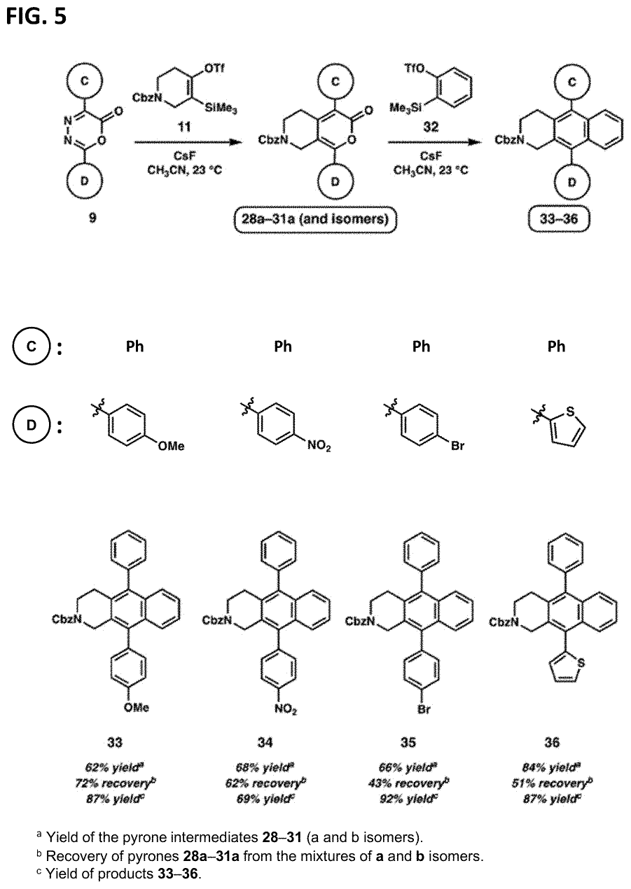

Furthermore, FIG. 5 illustrates the installation and diversification of quadrants C and D of PAHs 7 of the instant application. As noted earlier, in most known routes to 9,10-anthracene derivatives, the C and D rings are introduced through a double cross-coupling or by the double addition of an organometallic reagent, allowing for the formation of only symmetric products with limited functional group compatibility. However, the methods of the instant application allow for facile installation of many desirable aromatic and heteroaromatic rings in C and D quadrants of 7 via preparation of relevant oxadiazinones and their exposure to the desired cyclic or heterocyclic silyl triflates under the conditions described herein. For example, FIG. 5 demonstrates a series of differently substituted oxadiazinones 9 of many embodiments, prepared according to previously described methods, such that C is a phenyl and D is different aromatic moieties bearing various electron-donating and electron-withdrawing functional groups, as well as otherwise functional handles, and subjected to silyl triflate 11 under the relevant reaction conditions of some embodiments. Overall, in many embodiments, the rings C and D, may, independently, comprise one or more functionality selected from: an electron-donating functional group, including para-methoxyphenyl, an electron-withdrawing functional group, including para-NO₂, and a halogen atom, including F, Cl, Br, and I, heterocycles, including thiophene, alkenes, alkynes. For these examples, the desired sequence takes place to deliver pyrone isomers 28a/28b-31a/31b in yields ranging from 66 to 84%. Furthermore, re-subjection of isolated isomers 28a-31a (42 to 72% recovery of the single isomer from the mixture of isomers for depicted compounds) to benzyne precursor 32, under the same conditions of many embodiments, produces diverse PAHs 33-36 in good to excellent yields according to many embodiments. Here, para-bromide-containing PAH 35 can be further extended or decorated via a variety of known cross-coupling methods. Furthermore, incorporation of thiophene makes PAH 36 an attractive candidate for electronics applications, given the prevalence of thiophenes in organic electronics.

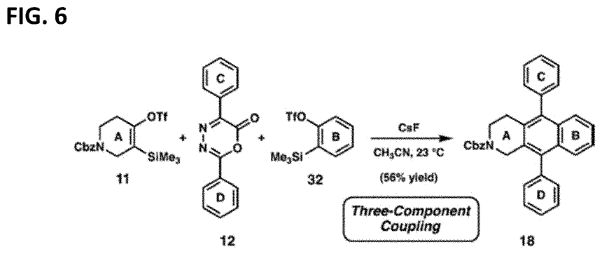

FIG. 6 illustrates a 3-component, “one pot” coupling of two different silyl triflates, for example 11 and 32, and oxadiazinone, e.g., 12, to produce PAH scaffold 18 according to some embodiments. In many such embodiments, operationally, CsF (or another Kobayashi elimination promoting reagent) is added to an equimolar solution of the three reactants. In one example, wherein 18 was produced from the simplest components—piperidyne, benzyne, and diphenyl oxadiazinone, 18 was obtained in 56% yield, along with its corresponding pyrone intermediate accounting for the remaining mass balance. Notably, in this example, the products of double piperidyne or benzyne addition were not observed, suggesting high selectivity of the methods of the instant application for the controlled formation of the PAHs and for the reaction of the two strained intermediates of the embodiments. Not to be bound by any theory, it is believed that, non-aromatic cylic silyl triflates, such as 11 of the instant application, undergo fluoride-mediated elimination to form the corresponding reactive alkyne more readily compared to benzyne precursor silyl triflate, such as, for example, 32, as a result of the lower strain energy associated with cyclic alkyne (e.g. 3,4-piperidyne) compared to benzyne (56). Accordingly, such judicious selection of the reaction components allows for the transformation of many embodiments to proceed by way of 4 consecutive pericyclic reactions to create 4 new C—C bonds and deliver a heterocyclic PAH scaffold 7 in one-pot.

### Triphenylene Scaffold PAHs

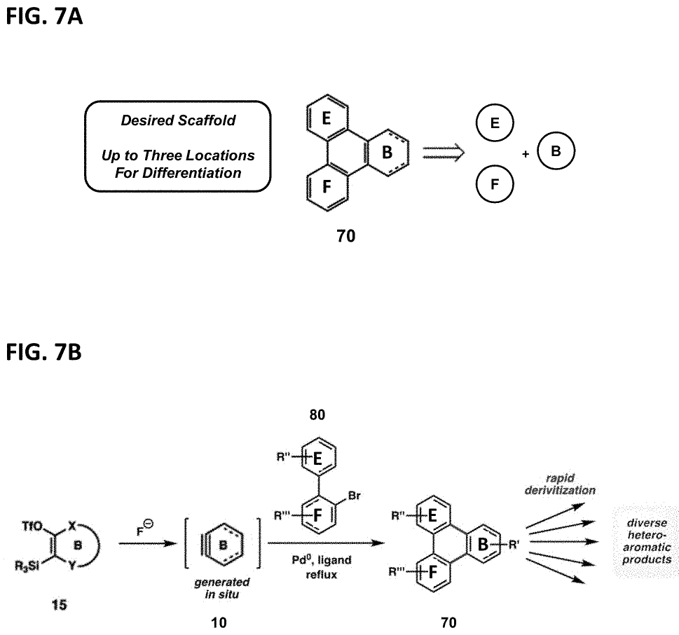

Alternatively, FIG. 7A depicts a triphenylene-based PAH scaffold (70) targeted by the synthetic methods of some other embodiments of the instant application. In many such embodiments, aromatic ring fragments E, F, and ring fragment B of scaffold 7 (wherein B is a either aromatic or non-aromatic and may contain a heteroatom) are united with formation of the central benzene ring and overall scaffold 70 (FIG. 7A). In many embodiments, 70 contains at least one nitrogen in ring B. More specifically, in many embodiments, formation of scaffold 70 via synthetic methods of the instant application enables access to a diverse range of triphenylene-based PAH scaffolds, with the possibility of accessing three structural locations (rings E, F, and B in FIGS. 7A and 7B) for differentiation. To this end, FIG. 7B provides the schematically depicted process for the formation of such scaffolds 70, wherein, according to many embodiments, reactive cyclohexyne or aryne 10 (building block B) is controllably generated in situ from the corresponding silyl triflate 15 (as already described herein), and cross-coupled with bromo-biaryl 80 (building blocks E and F) to produce heteroatom-containing triphenylene-based PAHs of the desired substitution and functionalization (i.e., with desired R′, R″, and R″′ in rings B, E, and F, respectively). In many embodiments, any or all of the thus installed R′, R″, and R″′ allow for further rapid derivatization to produce diverse heteroaromatic products.

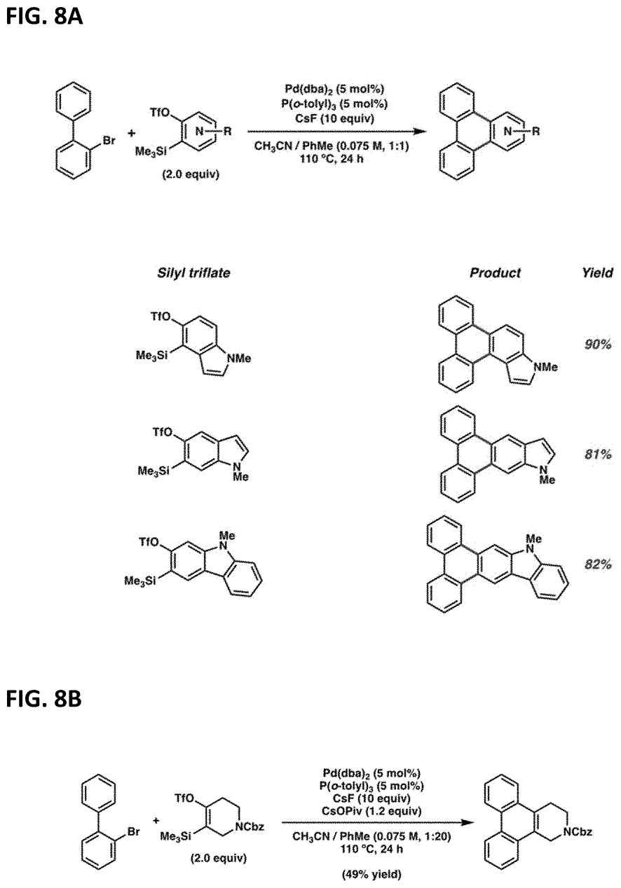

In many embodiments, the reaction conditions of the methods of the instant application comprise excess of silyl triflate 15 relative to bromo-biaryl 80. In some such embodiments 2 equivalents of silyl triflate are provided for each equivalent of bromo-biaryl. However, in some embodiments, only 1 equivalent of 15 relative to bromo-biaryl 80 is provided and such conditions still generate the desired PAH product, albeit with a lower yield. In many embodiments, the reaction conditions further comprise, 1 to 20 equivalents of CsF (or another Kobayashi elimination reagent), 5 to 100 mol % (but optimally 5 mol %) of Pd⁰ catalyst, the same amount of an appropriate ligand, and reflux conditions, including appropriately chosen solvent or solvent mixture, for 0.5 to 24 hours. In some such embodiments, the reaction conditions comprise: 1 equivalent of bromo-biaryl, 2 equivalents of cyclohexyne or aryne 10, 10 equivalents of CsF, 5 mol % Pd(dba)₂, 5 mol % P(o-tolyl)3, a 1:1 acetonitrile/toluene solvent mixture, wherein the concentration of bromo-biaryl is 0.075M, 110° C., and 24 hours. In some embodiments, especially wherein ring B is non-aromatic, it has been noticed that, unexpectedly, addition of CsOPiv significantly improves the overall yield of the desired PAH (FIG. 8B). Accordingly, in some embodiments, wherein it is desirable to have a PAH scaffold with non-aromatic rings, and wherein cyclohexyne or heterocyclohexyne is used in the cross-coupling reaction of embodiments, 1.2 equivalents of CsOPiv is added to the reaction mixture of the instantly disclosed methods. In some embodiments, the synthetic sequence for obtaining 70 depicted in FIG. 7B is performed stepwise, with sequential addition of components and reagents. In other embodiments, the synthetic sequence for obtaining 70 is performed in a one-pot fashion.

FIGS. 8A and 8B illustrate the scope and diversity of silyl triflate candidates 15 for use in the methods of the instant application and the resulting triphenylene scaffold PAHs. Here, FIG. 8A provides examples of diversification of hetero-aromatic B ring, while FIG. 8B provides an example where building block B is non-aromatic heterocyclohexyne, in accordance with many embodiments. More specifically, in many embodiments, as illustrated by FIGS. 8A and 8B, a great variety of aromatic and non-aromatic silyl triflates readily undergo Pd-catalyzed cross-coupling with bromo-biaryl, such as bromo-biphenyl, in the presence of Kobayashi elimination promoting CsF to produce useful new PAH architectures, including architectures featuring carbazole moiety, in good yields.

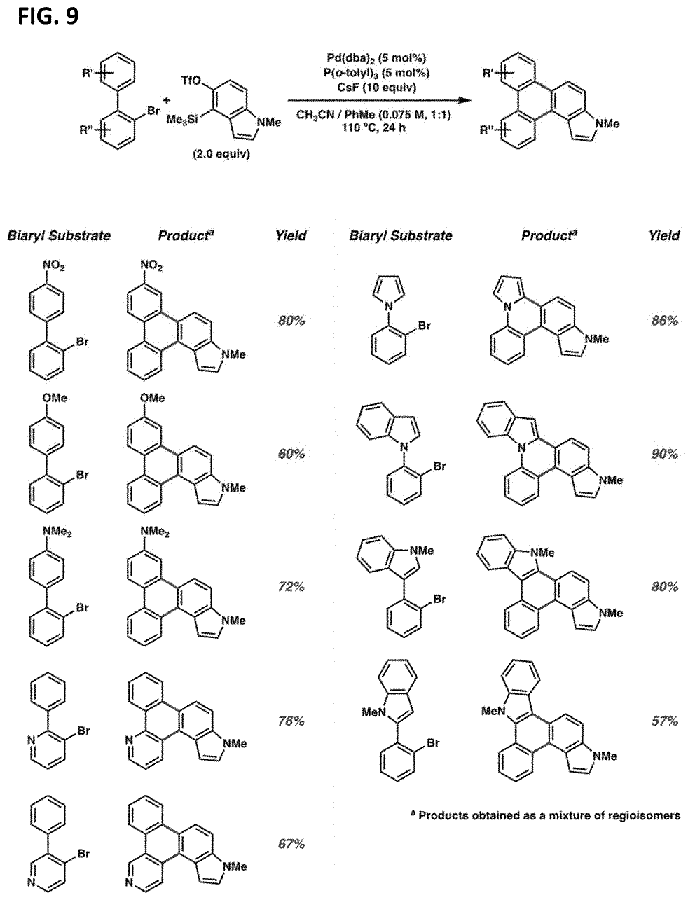

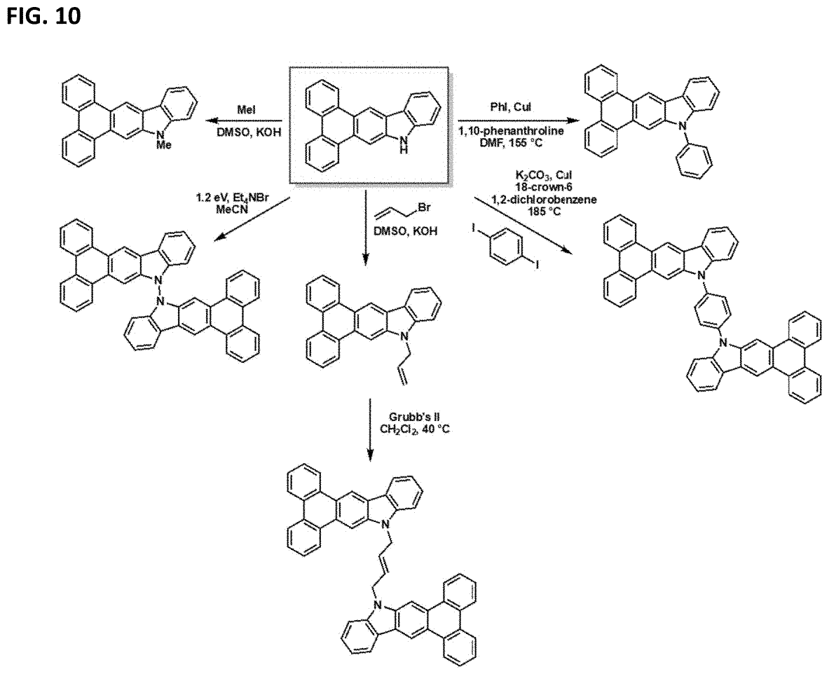

In addition, FIG. 9 illustrates the scope and diversity of bromo-biaryl substrates suitable for use in the methods of the instant application and the resulting triphenylene scaffold PAHs with diversified E and F rings, in accordance with many embodiments. More specifically, in many embodiments, as illustrated by FIG. 9, a variety of bromo-biaryls readily undergoes Pd-catalyzed cross-coupling with arynes, including hetero-arynes and, more specifically, aza-arynes, generated in situ from the corresponding silyl triflates via Kobayashi elimination, to produce diverse PAH architectures. The examples presented in FIG. 9 include bromo-biaryls with electron-donating and electron withdrawing functional groups, a heteroatom (e.g., N), and various aromatic architectures, all of which produce heteroatom-containing phenylene-based PAHs in good to excellent yields when reacted with, for example, indolyne (i.e., its silyl trifalte precursor). In some embodiments, the PAHs of embodiments are obtained as a mixture of stereoisomers, further expanding the architectural diversity.

FIG. 10 illustrates the potential for rapid diversification PAHs of the instant application according to many embodiments. In many embodiments, the diversification strategies illustrated in FIG. 10 can be applied to either 9,10-diphenylanthracene scaffolds 7 or triphenylene scaffolds 70, or any other similar scaffold comprising an available for decoration heteroatom. In this example, a carbazole-based PAH scaffold obtained via the cross-coupling method of the embodiments from bromo-biphenyl substrate and silyl triflate of carbazole is converted into a variety of complex structures in one to two simple and robust steps. More specifically, this scaffold's nitrogen atom can be easily methylated for protection, or phenylated for protection or improved electronic properties, or it can be used to dimerize the scaffold with or without inclusion of other moieties to obtain an even greater variety of complex useful organic architectures.

### Exemplary Embodiments

Experiments were conducted to demonstrate the capabilities of the methods and platforms in accordance with embodiments. These results and discussion are not meant to be limiting, but merely to provide examples of operative methods and platforms and their features.

### Examples Related to 9,10-Diphenylanthracene PAH Scaffolds

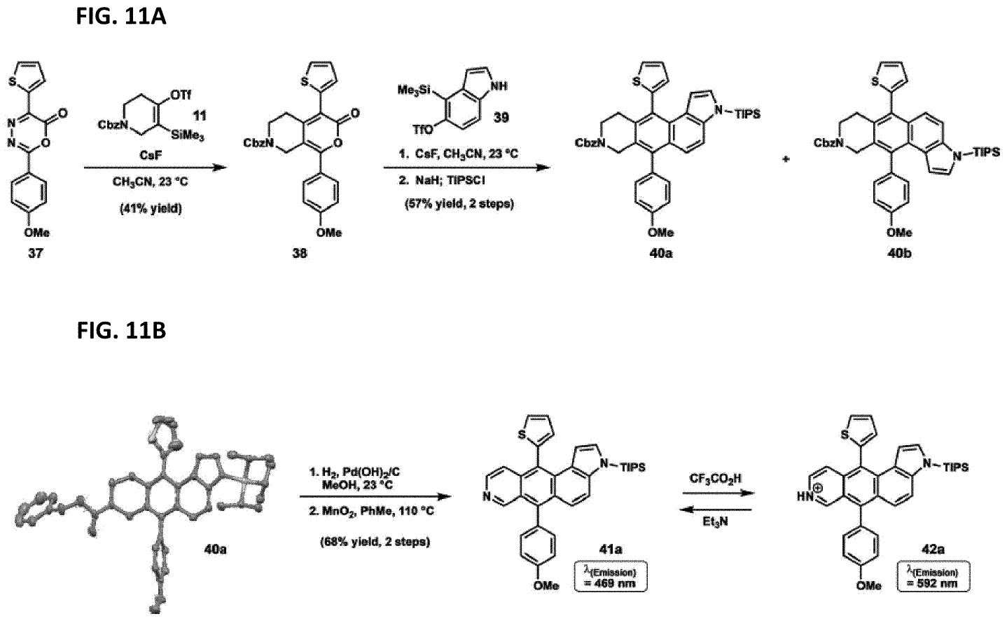

FIGS. 11A and 11B illustrate synthetic applications of the methods of the instant application, wherein complex, heteroatomic PAH scaffolds incorporating motifs commonly utilized in materials chemistry are accessed according to many embodiments. To this end, in one exemplary embodiment shown in FIG. 11A, oxadiazinone 37 (prepared from the corresponding hydrazide and glyoxylic acid) was first treated with silyl triflate 11 and CsF to furnish pyrone 38. Next, cycloaddition between 38 with silyl triflate 39 under similar conditions afforded the corresponding expected regioisomeric products of the DA/rDA sequence. Finally, silyl protection of the indole nitrogen provided separable isomers 40a and 40b. Notably, according to embodiments, compounds 40a and 40b bear all of thiophene, indole, pyridine, and para-methoxyphenyl motifs (i.e., four different aromatic groups on the four quadrants of substitution, three of which are heterocycles), and, therefore, represent a powerful demonstration of the modularity of methods and systems of embodiments, which allow facile and rapid access to compounds with four axes of substitution.

Furthermore, as depicted in FIG. 11B, removal of the Cbz-protecting group of 40a under hydrogenolysis conditions, followed by MnO₂-mediated oxidation produces 41a, which bears three heterocycles and a p-OMe-Ph motif. Notably, 41a exhibits pH-responsive fluorescence switching properties, wherein neutral 41a displays a blue fluorescence emission, whereas the protonated version, 42a, displays an orange fluorescence emission. Stimuli responsive materials, such as 42a obtained according to embodiments, are important for a host of materials-related applications such as pH fluorescence sensors (see, for example: X. Liu, et al., New J. Chem. 2017, 41, 10607-10612; and Q.-J. Ma, et al., Sens. Actuators B 2012, 166, 68-74, the disclosure of which are incorporated herein by reference) and solid-state fluorescent switches (for example, L. Tan, et al., J. Mater. Chem. C 2018, 6, 10270-10275, the disclosure of which is incorporated herein by reference).

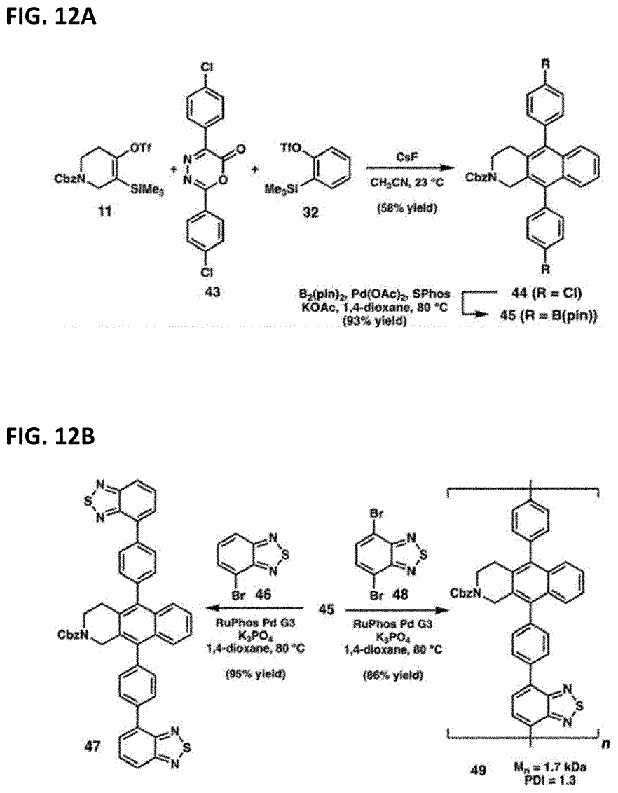

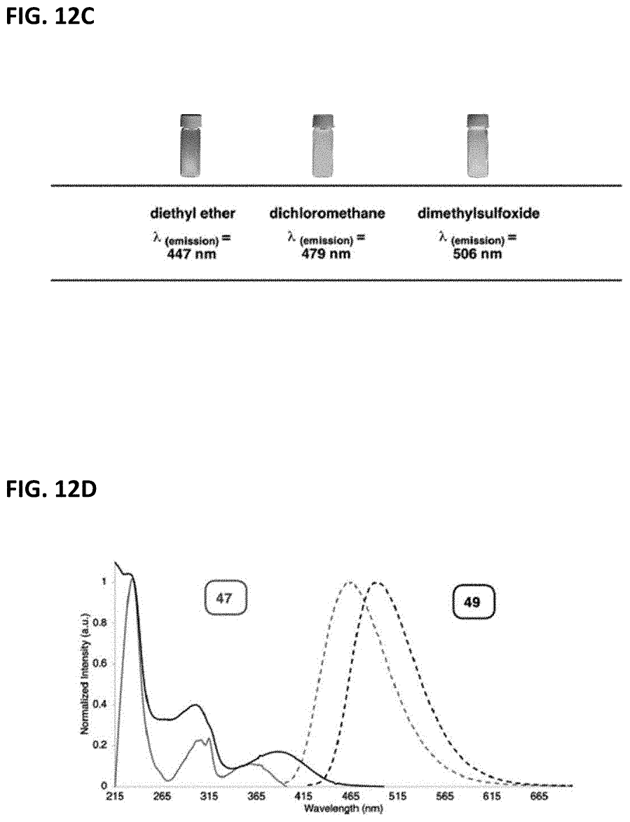

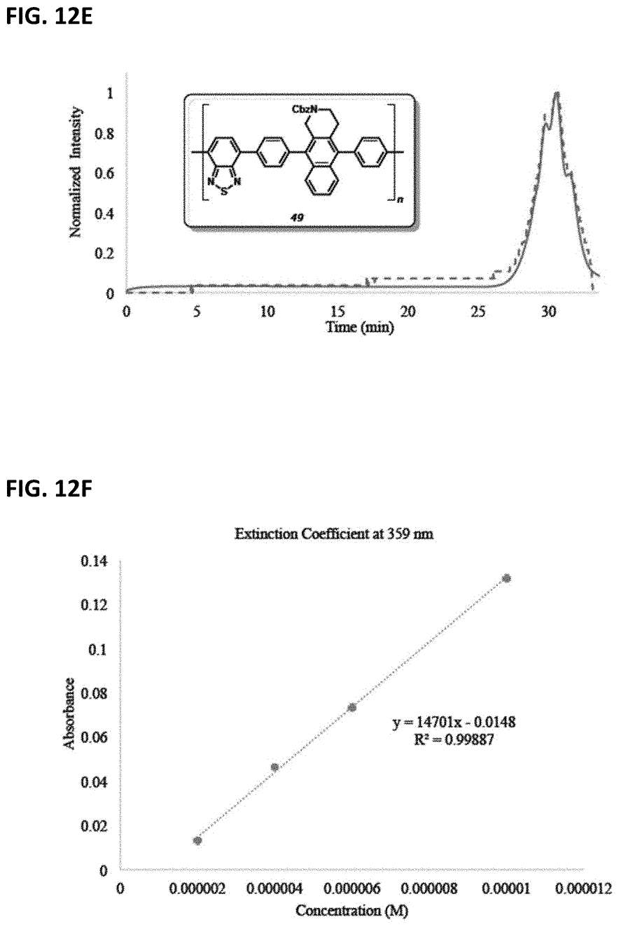

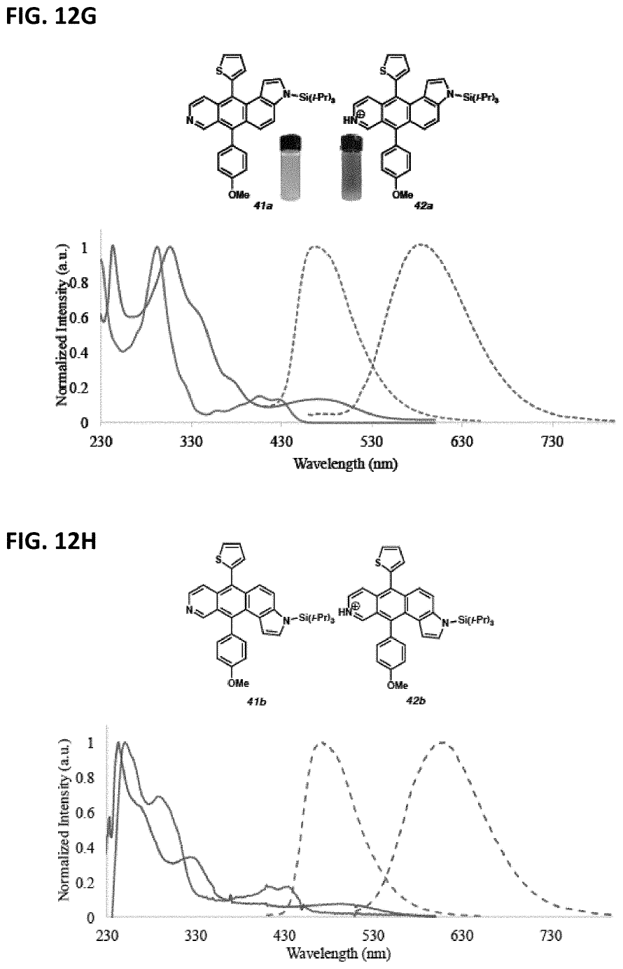

In addition FIGS. 12A and 12B illustrate a synthetic strategy according to embodiments to rapidly access monomeric and oligomeric donor-acceptor fluorophores. Specifically, first, the one-pot, 3-component coupling of silyl triflates 11 and 32 with dichlorooxadiazinone 43 (FIG. 12A) leads to the controlled formation of dichloride 44 in 58% yield. Next, 44 readily undergoes Pd-catalyzed borylation to give (bis)boronic ester 45, which is now available for further manipulation.

Accordingly, FIG. 12B shows the various transformation of borylated PAH 45 formed in accordance with various embodiments. For example, a Suzuki-Miyaura cross-coupling of 45 with mono-brominated benzothiadiazole 46 affords donor-acceptor fluorophore 47 in excellent yield (95% yield). Notably, 47 was found to be solvatochromic (see C. Reichardt, Chem. Rev. 1994, 94, 2319-2358, the disclosure of which is incorporated herein by reference), indicative of a donor-acceptor system (FIG. 12C). In another example, 45 is employed as a building block for polymer synthesis, wherein Suzuki-Miyaura cross-coupling polymerization (K.-B. Seo, et al., J. Am. Chem. Soc. 2018, 140, 4335-4343, the disclosure of which is incorporated herein by reference) of bis(boronate) 45 with bis-brominated, 4,7-dibromobenzothiadiazole 48 to provide donor-acceptor oligomer 49. Notably, oligomer 49 of embodiments was found to have a polydispersity index (PDI) of 1.3 and a number average molecular weight (Mn) of 1.7 kDa. Furthermore, the donor-acceptor oligomer 49 displays a red-shifted absorbance and emission relative to 47 with a longest-wavelength absorption maximum of I=391 nm and an emission maximum of I=491 nm (FIG. 12D). The chromatographic shifts can be attributed to the extended conjugation present in oligomer 49 compared to 47. In addition, FIG. 12E provides normalized UV trace (solid line) and refractive index trace (dashed line) for polymer 49; FIG. 12F shows extinction coefficient of 47 at 359 nm in tetrahydrofuran (THF); FIG. 12G demonstrates UV/Vis (solid lines) and emission (dashed lines) of neutral 41a and protonated 42a (shifted to the right lines) in dichloromethane; and FIG. 12H provides UV/Vis (solid lines) and emission (dashed lines) of neutral 41b (blue lines) and protonated 42b (shifted to the right lines) in dichloromethane. Together, these results demonstrate how a common adduct obtained according to methods of embodiments (i.e., 44) can be used to rapidly access donor-acceptor monomers and oligomers displaying differing photophysical properties.

### Examples Related to Triphenylene PAH Scaffolds

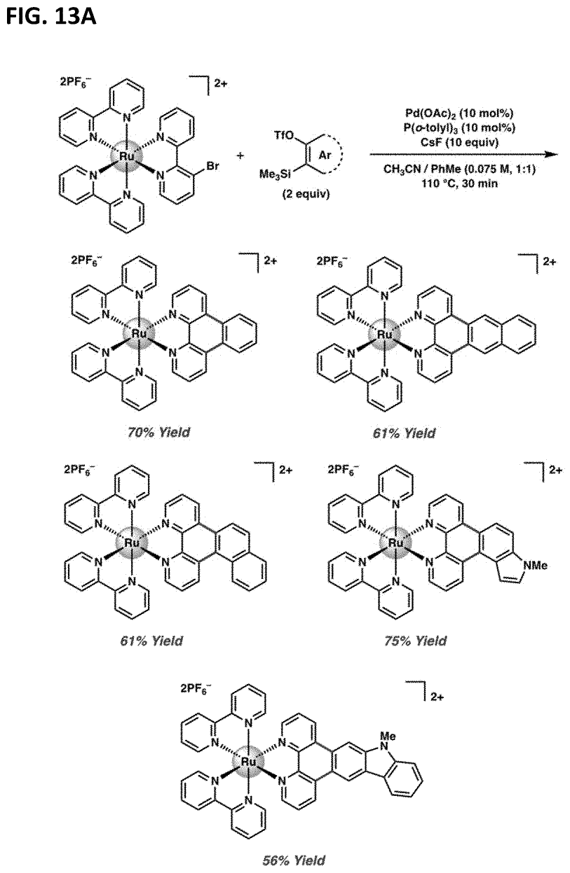

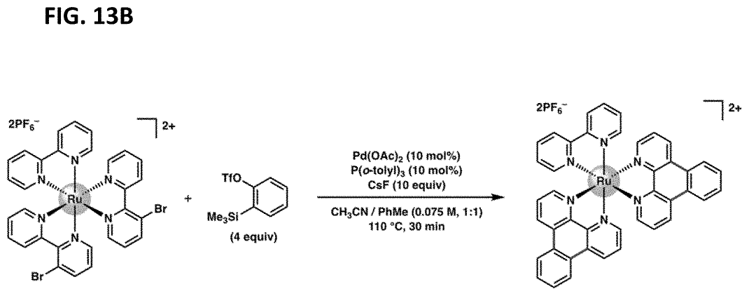

FIGS. 13A and 13B illustrate and provide scope for silyl triflate annulations, including double annulation, on Ruthenium organometallic complexes according to some embodiments. Ru(II) polypyridyl complexes, exemplified by [Ru(bpy)]²⁺, have seen broad utility as photosensitizer molecules in a wide variety of light-based applications. This includes photoredox catalysis (see, for example: Chem. Rev. 2013, 113, 5322-5363, the disclosure of which is incorporated herein by reference), solar energy conversion (see, for example, J. Photochem. Photobiol., C 2003, 4, 145-153, the disclosure of which is incorporated herein by reference), luminescence sensing (for example: Coord. Chem. Rev. 2000, 205, 201-228, the disclosure of which is incorporated herein by reference), and more recently, photodynamic therapy (see, for example: Chem. Rev. 2019, 119, 797-828, the disclosure of which is incorporated herein by reference). Synthetic methods for direct manipulation of these ligand-metal complexes are sparse, thus precluding the ability to rapidly diversify the Ru(bpy)₃ scaffold, Crucially, it has recently been demonstrated that expanded ligand aromaticity can result in enhanced photosensitizer activity. Accordingly, diversification of Ruthenium's bpy ligands via direct □-extension may expand the scope of its applications even further. FIG. 13A provides examples of such facile and rapid diversification, wherein a tris(bipyridine)ruthenium(II) complex has one of its bipyridine ligands replaced with a bromo-bipyridine, making it available for cross-coupling with a silyl triflate of embodiments according to the methods of the instant application. FIG. 13A, thus, provides a number of examples, wherein one of the Ru's bpy ligands is extended into various triphenylene-type PAH scaffolds with different geometries and inclusions of additional heteroatoms (e.g., N). FIG. 13B shows that more than one ligand can be converted at a time according to embodiments. Accordingly, and notably, the methods of the instant application uniquely allow to directly enhance and diversify transition metal containing complexes with transition metal catalyzed processes. Also of note, it has been found, that, unexpectedly, when the reaction conditions for the embodiments involving Ru complexes comprise Pd(OAc)₂ catalyst, rather than Pd(dba)₂, the product yields are much improved. In many embodiments, organometallic complexes comprising a transition metal selected from: Co, Ir, Rh, Ni, Pd, Pt, Zn, Cu, Fe, Mn, Os, can all be enhanced with the methods of the instant application.

### Experimental Section

### Materials and Methods Related to 9,10-Diphenylanthracene PAH Scaffolds.

Unless stated otherwise, reactions were conducted in flame-dried glassware under an atmosphere of nitrogen using anhydrous solvents (freshly distilled or passed through activated alumina columns). All commercially obtained reagents were used as received unless otherwise specified. Cesium fluoride (CsF) and manganese (IV) oxide (MnO₂) were obtained from Strem Chemicals. Benzyl 4-(trifluoromethylsulfonyloxy)-3-(trimethylsilyl)-5,6-dihydropyridine-1(2H)-carboxylate (11), Garg 4,5-indolyne precursor (precursor to 21), 2-(trimethylsilyl)phenyl trifluoromethanesulfonate (32), sodium hydride (NaH, 60% dispersion in mineral oil), palladium hydroxide on carbon (20% wt. loading, Pd(OH)₂/C), bis(pinacolato) diboron (B₂(pin)₂), SPhos, and 4-bromo-benzothiadiazole (46) were obtained from Sigma Aldrich. Triisopropylsilyl chloride (TIPSCI) 4,7-dibromo-benzothiadiazole (48), and RuPhos-Pd-G3 were obtained from Combi-Blocks. 1-(Trimethylsilyl)-2-naphthyl trifluoromethanesulfonate (precursor to 19) was obtained from TCI America. Potassium acetate (KOAc) was obtained from Fisher Scientific and ground up and dried in an oven overnight prior to use. Potassium phosphate (K₃PO₄) was obtained from Acros. TIPSCI was freshly distilled before use. 1,4-Dioxane was dried overnight with 10% wt/wt 5 Å molecular sieves and sparged with nitrogen for 30 min immediately before use. Reaction temperatures were controlled using an IKAmag temperature modulator and, unless stated otherwise, reactions were performed at room (i.e, ambient) temperature (approximately 23° C.). Thin layer chromatography (TLC) was conducted with EMD gel 60 F254 pre-coated plates (0.25 mm) and visualized using a combination of UV light, anisaldehyde, and potassium permanganate staining. Silicycle Siliaflash P60 (particle size 0.040-0.063 mm) was used for flash column chromatography. For some chromatographic purifications, an automated Biotage Isolera™ with SNAP Ultra™ cartridges, Teledyne Isco CombiFlash® with RediSep Rf cartridges, or Yamazen Smart Flash AKROS with ELS detector and Universal columns were used. ¹H-NMR spectra were recorded on Bruker spectrometers (at 400, 500, and 600 MHz) and are reported relative to the residual solvent signal. Data for ¹H-NMR spectra are reported as follows: chemical shift (δ ppm), multiplicity, coupling constant (Hz) and integration. ¹³C-NMR spectra were recorded on Bruker spectrometers (at 100 and 125 MHz) and are reported relative to the residual solvent signal. Data for ¹³C-NMR spectra are reported in terms of chemical shift and, when necessary, multiplicity, and coupling constant (Hz). IR spectra were obtained on a Perkin-Elmer UATR Two FT-IR spectrometer and are reported in terms of frequency of absorption (cm⁻¹). Uncorrected melting points were measured using a Digimelt MPA160 melting point apparatus. DART-MS spectra were collected on a Thermo Exactive Plus MSD (Thermo Scientific) equipped with an ID-CUBE ion source and a Vapur Interface (lonSense Inc.). Both the source and MSD were controlled by Excalibur software v. 3.0. The analyte was spotted onto OpenSpot sampling cards (IonSense Inc.) using CDCl₃ as the solvent. Ionization was accomplished using UHP He (Airgas Inc.) plasma with no additional ionization agents. The mass calibration was carried out using Pierce LTQ Velos ESI (+) and (−) Ion calibration solutions (Thermo Fisher Scientific). Separation of compounds 40a and 40b was carried out by Scott Virgil at California Institute of Technology on a Jasco 2000 SFC (supercritical fluid chromatography) Preparative System using a Chiral Technologies AD-H column. UV-Vis spectra were recorded using an JASCO C-770 UV-Visible/NIR spectrophotometer. Fluorescence spectra were recorded using a Horiba Instruments PTI Quanta Master Series Fluorometer. The UV-Vis and fluorescence spectra were recorded using a 1-cm quartz cuvette, with freshly distilled tetrahydrofuran (THF), methylene chloride (DCM), diethyl ether, and acetonitrile. Gel permeation chromatography (GPC) was conducted on a Shimadzu HPLC Prominence-i system equipped with a UV detector, Wyatt DAWN Heleos-II Light Scattering detector, Wyatt Optilab T-rEX RI detector, one MZ-Gel SDplus guard column, and two MZ-Gel SDplus 100 Å 5 μm 300×8.0 mm columns. Tetrahydrofuran (THF) at 40° C. was used as the eluent (flow rate: 0.70 mL/min). For polymer 49 near-monodisperse poly(styrene) standards (Polymer Laboratories) were employed for calibration and molecular weights were calculated from refractive index.

Oxadiazinone SI-13, hydrazones SI-3, SI-5 and SI-8 (see M. L. Tintas, et al., J. Mol. Struct. 2014, 1058, 106-113, the disclosure of which is incorporated herein by reference), and silyl triflates SI-15 (A. S. Devlin, et al., Chem. Sci. 2013, 4, 1059-1063, the disclosure of which is incorporated herein by reference) and SI-16 (T. K. Shah, et al., J. Am. Chem. Soc. 2016, 138, 4948-4954, the disclosure of which is incorporated herein by reference) are known compounds and were prepared following literature procedures. ¹H-NMR spectral data matched those reported in the literature. Silyl triflate precursors to 17, 19, 21, and 25 are all commercially available from Sigma-Aldrich (www.sigmaaldrich.com) or TCI (www.tcichemicals.com). The Sigma-Aldrich product numbers are as follows: 2-(trimethylsilyl)phenyl trifluoromethanesulfonate (precursor to 17): 470430; Garg 4,5-indolyne precursor (precursor to 21): 795569; benzyl 4-(trifluoromethylsulfonyloxy)-3-(trimethylsilyl)-5,6-dihydropyridine-1(2H)-carboxylate (precursor to 25): 803928. The TCI product number for 1-(trimethylsilyl)-2-naphthyl trifluoromethanesulfonate (precursor to 19) is T2465.

### Experimental Procedures.

A. Syntheses of Oxadiazinones.

General Procedure A (Preparation of Oxadiazinone 12 is Used as an Example of Embodiments).

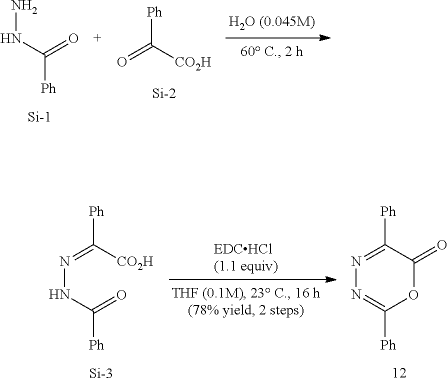

Oxadiazinone 12. To a 60° C. solution of benzohydrazide SI-1 (7.00 g, 51.4 mmol, 1.00 equiv) in deionized water (640 mL) and open to air, was added a solution of glyoxylic acid SI-2 (7.72 g, 51.5 mmol, 1.00 equiv) dissolved in deionized water (645 mL) dropwise over 2 h. After stirring for 2.5 h at 60° C., the reaction flask was cooled to 23° C. and the products were allowed to crystallize overnight. Filtration afforded hydrazine SI-3 (15.5 g crude mass) as a white solid, which was carried forward to the next reaction without purification.

To a solution of hydrazine SI-3 (15.5 g, 57.8 mmol, 1.00 equiv) in THF (580 mL) was added EDC.HCl (12.2 g, 63.6 mmol, 1.10 equiv). After stirring at 23° C. for 16.5 h, the reaction was concentrated under reduced pressure. The crude reaction mixture was transferred to a separatory funnel using Et₂O (200 mL), H₂O (200 mL), and a minimal amount of THF to dissolve precipitates (150 mL). The layers were then separated and the organic layer was washed successively with deionized water (3×100 mL) and brine (1×100 mL), dried over MgSO₄, filtered, and concentrated under reduced pressure to yield oxadiazinone 12 (10.1 g, 78% yield based on SI-1) as a yellow solid. Oxadiazinone 12: mp 137-138° C.; R_{f} 0.41 (9:1 EtOAc:MeOH); ¹H-NMR (400 MHz, CDCl₃): δ 8.36-8.32 (m, 2H), 8.32-8.27 (m, 2H), 7.68-7.62 (m, 1H), 7.60-7.49 (m, 5H); ¹³C-NMR (100 MHz, CDCl₃): δ 157.8, 153.0, 148.4, 133.9, 132.3, 131.2, 129.3, 129.2, 128.9, 128.4, 127.7; IR (film): 3061, 1685, 1578, 1480, 1257 cm⁻¹; HRMS-APCI (m/z) [M+H]⁺ calcd for C₁₅H₁₁N₂O₂⁺, 251.0815; found, 251.0797. Melting point matched those previous reported (see, W. Steglich, et al., Synthesis 1977, 252-253, the disclosure of which is incorporated herein by reference).

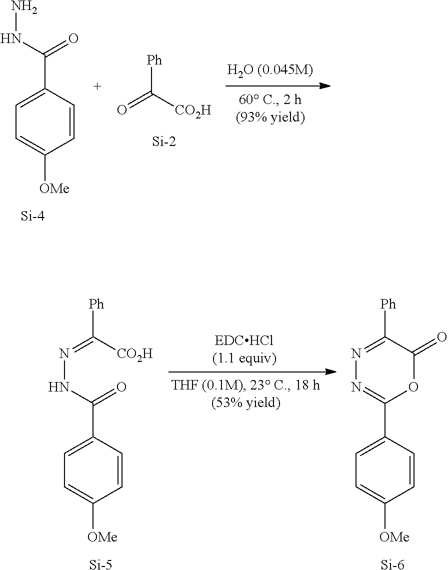

Oxadiazinone SI-6. Followed General Procedure A using hydrazide SI-4 (3.00 g, 18.1 mmol) to afford oxadiazinone SI-6 (2.00 g, 49% yield over two steps) as a yellow solid after recrystallization from hot EtOAc. Oxadiazinone SI-6: mp 178-180° C.; R_{f} 0.46 (9:1 EtOAc:MeOH); ¹H-NMR (500 MHz, CDCl₃): δ 8.33-8.29 (m, 2H), 8.26-8.21 (m, 2H), 7.57-7.53 (m, 1H), 7.53-7.48 (m, 2H), 7.06-7.02 (m, 2H), 3.91 (s, 3H); ¹³C-NMR (125 MHz, CDCl₃): δ 164.3, 157.9, 152.0, 148.6, 131.9, 131.4, 130.5, 129.0, 128.7, 119.7, 114.8, 55.8; IR (film): 3075, 2846, 1763, 1604, 1258 cm⁻¹; HRMS-APCI (m/z) [M+H]⁺ calcd for C₁₆H₁₃N₂O₃⁺, 281.0921; found, 281.0916. IR and HRMS matched those previously reported (see M. Christl, et al., Chem. Ber. 1985, 118, 2940-2973, the disclosure of which is incorporated herein by reference).

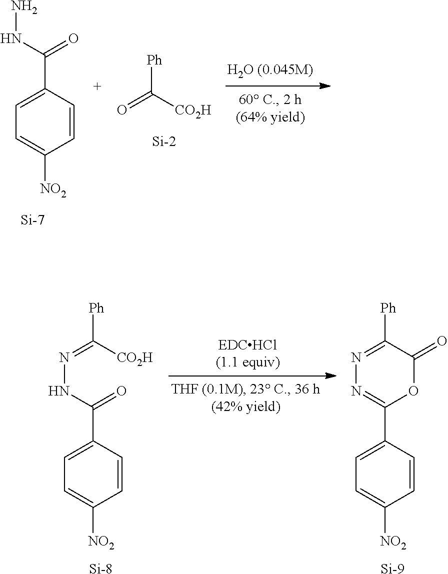

Oxadiazinone SI-9. Followed General Procedure A using hydrazide SI-7 (3.00 g, 16.6 mmol) to afford oxadiazinone SI-9 (1.32 g, 27% yield over two steps) as a yellow solid after recrystallization from hot EtOAc. Oxadiazinone SI-9: mp 220-224° C.; R_{f} 0.58 (9:1 EtOAc:MeOH); ¹H-NMR (500 MHz, CDCl₃): δ 8.48 (d, J=8.8, 2H), 8.41 (d, J=8.8, 2H), 8.35 (d, J=7.9, 2H), 7.62 (dd, J=7.3, 7.3, 1H), 7.54 (dd, J=7.6, 7.6, 2H); ¹³C-NMR (125 MHz, CDCl₃): δ 155.8, 153.9, 150.9, 147.5, 133.4, 132.9, 130.7, 129.4, 129.3, 129.0, 124.4; IR (film): 3079, 1775, 1563, 1521, 1350 cm⁻¹; HRMS-APCI (m/z) [M+H]⁺ calcd for C₁₅H₁₀N₃O₄⁺, 296.0658; found, 296.0666. Melting point and IR spectrum matched those previously reported (see E. Feineis, et al., Chem. Ber. 1993, 126, 1743-1748, the disclosure of which is incorporated herein by reference).

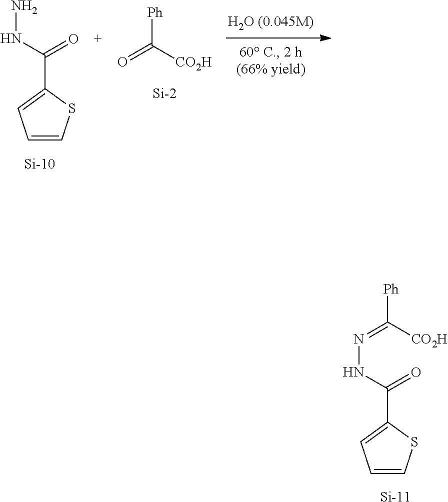

Hydrazone SI-11. Followed General Procedure A using hydrazide SI-10 (3.00 g, 21.1 mmol) to afford hydrazone SI-11 (3.80 g, 66% yield) as a white solid. Hydrazone SI-11: mp 186-187° C.; R_{f} 0.24 (9:1 EtOAc:MeOH); ¹H-NMR (600 MHz, DMSO-d₆, 55° C.): δ 12.14 (br s, 1H), 7.97-7.93 (m, 2H), 7.76-7.72 (m, 2H), 7.48-7.44 (m, 3H), 7.24 (dd, J=4.9, 3.8, 1H); ¹³C-NMR (151 MHz, DMSO-d₆, 65° C.): δ 164.5, 163.2, 134.2, 129.4, 128.9, 128.5, 128.2, 127.9, 127.8, 127.09, 127.05; IR (film): 3247, 3109, 3029, 1699, 1404 cm⁻¹; HRMS-APCI (m/z) [M+H]⁺ calcd for C₁₃H₁₁N₂O₃S⁺, 275.0485; found, 275.0475.

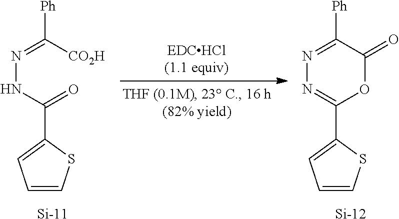

Oxadiazinone SI-12. Followed General Procedure A using hydrazone SI-11 (3.00 g, 10.9 mmol) to afford oxadiazinone SI-12 (2.30 g, 82% yield) as a yellow solid after recrystallization from hot Et₂O. Oxadiazinone SI-12: mp 142-143° C.; R_{f} 0.70 (9:1 EtOAc:MeOH); 1H-NMR (500 MHz, CDCl₃): δ 8.32-8.28 (m, 2H), 8.00 (dd, J=3.8, 1.2, 1H), 7.72 (dd, J=5.0, 1.2, 1H), 7.58-7.54 (m, 1H), 7.52-7.48 (m, 2H), 7.22 (dd, J=5.0, 3.8, 1H); ¹³C-NMR (125 MHz, CDCl₃): δ 155.0, 152.1, 147.8, 134.4, 133.2, 132.1, 131.3, 130.7, 129.1, 128.9, 128.8; IR (film): 3096, 1767, 1561, 1422, 1152 cm⁻¹; HRMS-APCI (m/z) [M+H]⁺ calcd for C₁₃H₉N₂O₂S⁺, 257.0379; found, 257.0371.

B. Syntheses of Pyrone Intermediates.

General Procedure B (Preparation of Pyrones 13a and 13b is Used as an Example of Embodiments).

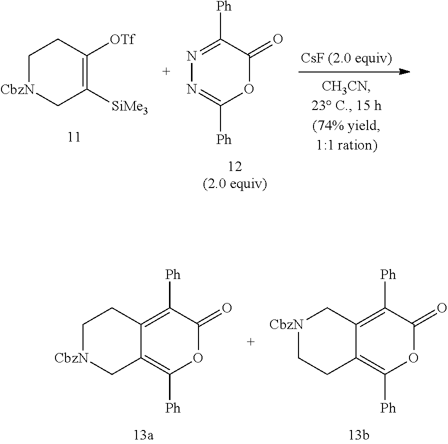 [SMILES: C[Si](C)(C)C1=C(OS(=O)(=O)C(F)(F)F)CCN(C(=O)OCc2ccccc2)C1 ; O=c1oc(-c2ccccc2)nnc1-c1ccccc1 ; O=C(OCc1ccccc1)N1CCc2c(c(-c3ccccc3)oc(=O)c2-c2ccccc2)C1 ; O=C(OCc1ccccc1)N1CCc2c(-c3ccccc3)oc(=O)c(-c3ccccc3)c2C1]

Pyrones 13a and 13b. To a stirred solution of silyl triflate 11 (66 mg, 0.15 mmol, 1.0 equiv) and oxadiazinone 12 (75 mg, 0.30 mmol, 2.0 equiv) in acetonitrile (1.5 mL) was added CsF (46 mg, 0.30 mmol, 2.0 equiv) in one portion. The reaction was purged with nitrogen for ten minutes before being sealed with a Teflon cap and left to stir at 23° C. After 15 h, the reaction mixture was filtered through celite (monster pipette, ˜4 cm tall) with EtOAc (˜10 mL) as the eluent and then concentrated under reduced pressure. Purification of the crude material via flash chromatography (Yamazen, 8 g SiO₂, 100% hexanes→3:2 hexanes:EtOAc) afforded pyrones 13a and 13b (48.4 mg, 74% yield) as a light yellow foam. Pyrones 13a and 13b: R_{f} 0.13 (5:1 hexanes:EtOAc); ¹H-NMR (500 MHz, CD₃CN, 60° C.): δ 7.66-7.59 (m, 5H), 7.57-7.52 (m, 6H), 7.50-7.41 (m, 7H), 7.37-7.28 (m, 12H), 5.07 (s, 4H), 4.53 (s, 2H, 13a), 4.35 (s, 2H, 13b), 3.57-3.49 (m, 4H), 2.82 (t, J=6.4, 2H, 13b), 2.72 (t, J=6.5, 2H, 13a); HRMS-APCI (m/z) [M+H]⁺ calcd for C₂₈H₂₄NO₄⁺, 438.1700; found, 438.1683.

General Procedure C (Preparation of Pyrone 13a is Used as an Example of Embodiments).

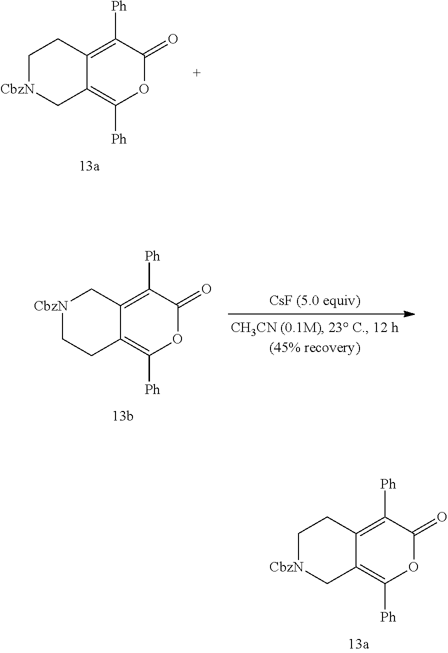 [SMILES: O=C(OCc1ccccc1)N1CCc2c(c(-c3ccccc3)oc(=O)c2-c2ccccc2)C1 ; O=C(OCc1ccccc1)N1CCc2c(-c3ccccc3)oc(=O)c(-c3ccccc3)c2C1 ; O=C(OCc1ccccc1)N1CCc2c(c(-c3ccccc3)oc(=O)c2-c2ccccc2)C1]

Pyrone 13a. To a solution of silyl triflates 13a and 13b (66 mg, 0.15 mmol, 1:1 ratio of regioisomers, 1.0 equiv) in acetonitrile (0.4 mL) was added CsF (57 mg, 0.37 mmol, 5.0 equiv) in one portion. The reaction was purged with nitrogen for ten minutes before being left to stir at 23° C. After 12 h, the reaction mixture was filtered through celite (monster pipette, ˜4 cm tall) with EtOAc (˜10 mL) as the eluent and then concentrated under reduced pressure. Purification of the crude material via flash chromatography (Isco, 12 g SiO₂, 100% hexanes→100% EtOAc) afforded pyrone 13a (29.8 mg, 45% recovery) as a light yellow foam. Pyrone 13a: R_{f} 0.13 (5:1 hexanes:EtOAc); ¹H-NMR (500 MHz, CD₃CN, 60° C.): δ 7.62-7.53 (m, 5H), 7.50-7.30 (m, 10H), 5.07 (s, 2H), 4.53 (s, 2H), 3.52 (t, J=6.4, 4H), 2.72 (t, J=6.4, 2H); ¹³C-NMR (125 MHz, CD₃CN): δ 162.6, 156.2, 152.5, 138.4, 135.3, 133.1, 131.6, 131.3, 130.0, 129.9, 129.7, 129.6, 129.3, 129.1, 128.8, 125.1, 118.3, 113.5, 68.0, 42.3, 42.1, 28.2; IR (film): 3062, 3029, 2952, 1700, 1234 cm⁻¹; HRMS-APCI (m/z) [M+H]³⁰ calcd for C₂₈H₂₄NO₄⁺, 438.1700; found, 438.1691.

Note, that substantial amount of insoluble white solid was formed during the reaction. Not to be bound by any theory, it is hypothesized that isomer 13b decomposes under the reaction conditions to give this amorphous polymeric material. The mechanism of decomposition is not known at this time. % recovery is defined as follows:

$$\% recovery = \frac{pure iosmer mass}{initial mixture mass} \times 100.$$

For example:

$$\frac{39.8 mg}{66.0 mg} \times 100 = 45 \% recovery .$$

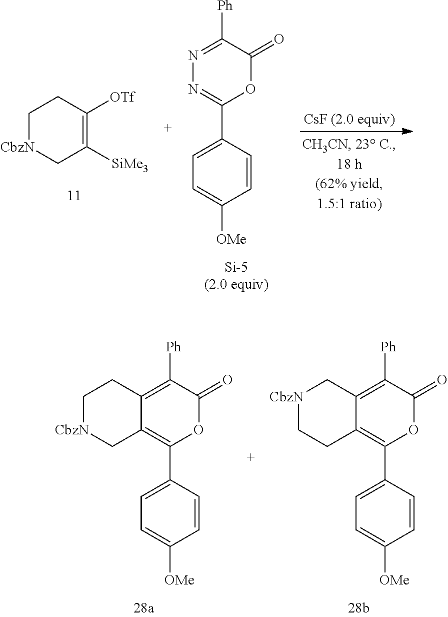 [SMILES: C[Si](C)(C)C1=C(OS(=O)(=O)C(F)(F)F)CCN(C(=O)OCc2ccccc2)C1 ; COc1ccc(-c2nnc(-c3ccccc3)c(=O)o2)cc1 ; COc1ccc(-c2oc(=O)c(-c3ccccc3)c3c2CN(C(=O)OCc2ccccc2)CC3)cc1 ; COc1ccc(-c2oc(=O)c(-c3ccccc3)c3c2CCN(C(=O)OCc2ccccc2)C3)cc1]

Pyrones 28a and 28b. Followed General Procedure B using silyl triflate 11 (66 mg, 0.15 mmol, 1.0 equiv) afforded pyrones 28a and 28b (62% yield, average of two experiments) as a yellow foam. Pyrones 28a and 28b: R_{f} 0.52 (1:1 hexanes:EtOAc); ¹H-NMR (500 MHz, CD3CN, 60° C):δ 7.60-7.52 (m, 5H), 7.50-7.39 (m, 6H), 7.37-7.28 (m, 11H), 7.18-7.13 (m, 2H), 7.09-7.04 (m, 4H), 5.09-5.04 (m, 4H), 4.55-4.51 (m, 3H), 4.34 (s, 1H), 3.89-3.86 (m, 7H), 3.56-3.48 (m, 4H), 2.95 (t, J=6.5, 2H), 2.71 (t, J=6.5, 2H); HRMS-APCI (m/z) [M+H]⁺ calcd for C₂₉H₂₆NO₅⁺, 468.1806; found, 468.1774.

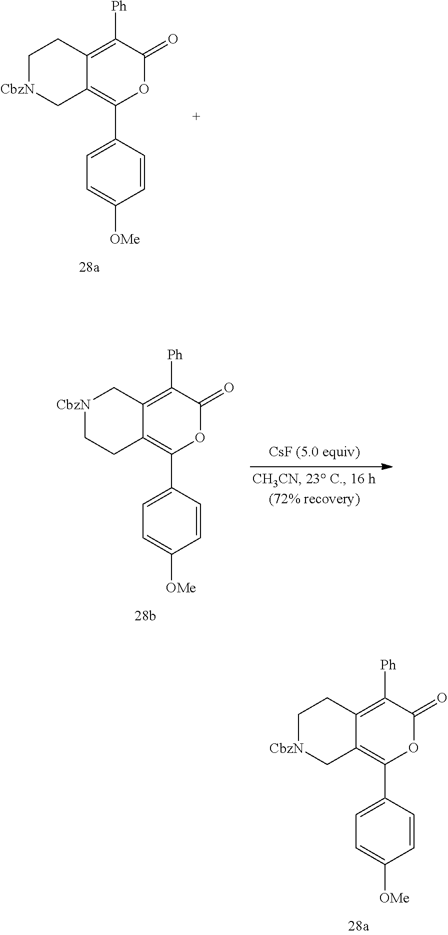 [SMILES: COc1ccc(-c2oc(=O)c(-c3ccccc3)c3c2CN(C(=O)OCc2ccccc2)CC3)cc1 ; COc1ccc(-c2oc(=O)c(-c3ccccc3)c3c2CCN(C(=O)OCc2ccccc2)C3)cc1 ; COc1ccc(-c2oc(=O)c(-c3ccccc3)c3c2CN(C(=O)OCc2ccccc2)CC3)cc1]

Pyrone 28a. Followed General Procedure C using pyrones 28a and 28b (90 mg, 0.193 mmol, 2.4:1 ratio of regioisomers) afforded pyrone 28a (65.0 mg, 72% recovery) as a yellow foam. Pyrone 28a: R_{f} 0.52 (1:1 hexanes:EtOAc); ¹H-NMR (500 MHz, CD₃CN, 60° C.): δ 7.58-7.52 (m, 2H), 7.49-7.44 (m, 2H), 7.43-7.39 (m, 1H), 7.36-7.24 (m, 7H), 7.10-7.04 (m, 2H), 5.07 (s, 2H), 4.54 (s, 2H), 3.89 (s, 3H), 3.51 (t, J=6.4, 2H), 2.71 (t, J=6.4, 2H); ¹³C-NMR (125 MHz, CD₃CN, 60° C.): δ (22 of 23 signals observed) 162.62, 162.59, 156.2, 152.6, 138.4, 135.3, 131.5, 131.3, 129.6, 129.5, 129.2, 129.1, 128.8, 125.3, 124.4, 115.6, 112.8, 68.0, 56.5, 42.3, 42.1, 28.2; IR (film): 3032, 2937, 2837, 1697, 1255 cm⁻¹; HRMS-APCI (m/z) [M+H]⁺ calcd for C₂₉H₂₆NO₅⁺, 468.1806; found, 468.1784.

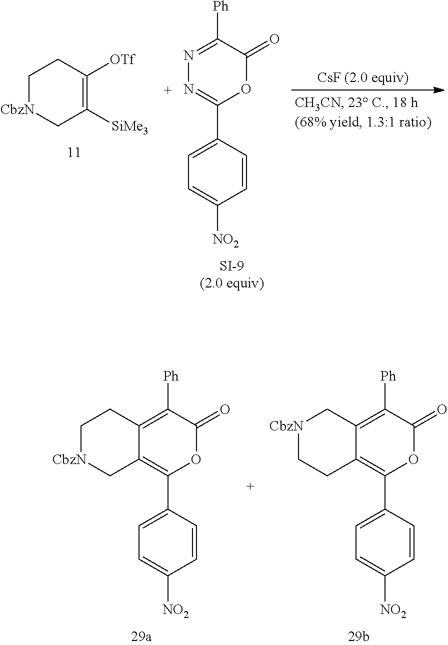 [SMILES: C[Si](C)(C)C1=C(OS(=O)(=O)C(F)(F)F)CCN(C(=O)OCc2ccccc2)C1 ; O=c1oc(-c2ccc([N+](=O)[O-])cc2)nnc1-c1ccccc1 ; O=C(OCc1ccccc1)N1CCc2c(c(-c3ccc([N+](=O)[O-])cc3)oc(=O)c2-c2ccccc2)C1 ; O=C(OCc1ccccc1)N1CCc2c(-c3ccc([N+](=O)[O-])cc3)oc(=O)c(-c3ccccc3)c2C1]

Pyrones 29a and 29b. Followed General Procedure B using silyl triflate 11 (66 mg, 0.15 mmol, 1.0 equiv) afforded pyrones 29a and 29b (68% yield, average of two experiments) as a yellow foam. Pyrones 29a and 29b: R_{f} 0.17 (7:3 hexanes:EtOAc); ¹H-NMR (500 MHz, CD₃CN, 70° C.): δ 8.36-8.28 (m, 4H), 7.89-7.79 (m, 4H), 7.52-7.41 (m, 7H), 7.37-7.27 (m, 13H), 5.07 (s, 4H), 4.53 (s, 2H, 29a), 4.37 (s, 2H, 29b), 3.58-3.52 (m, 4H), 2.84 (t, J=6.3, 2H, 29b), 2.75 (t, J=6.6, 2H 29a); HRMS-APCI (m/z) [M+H]⁺ calcd for C₂₈H₂₃N₂O₆⁺, 483.1551; found, 483.1538.

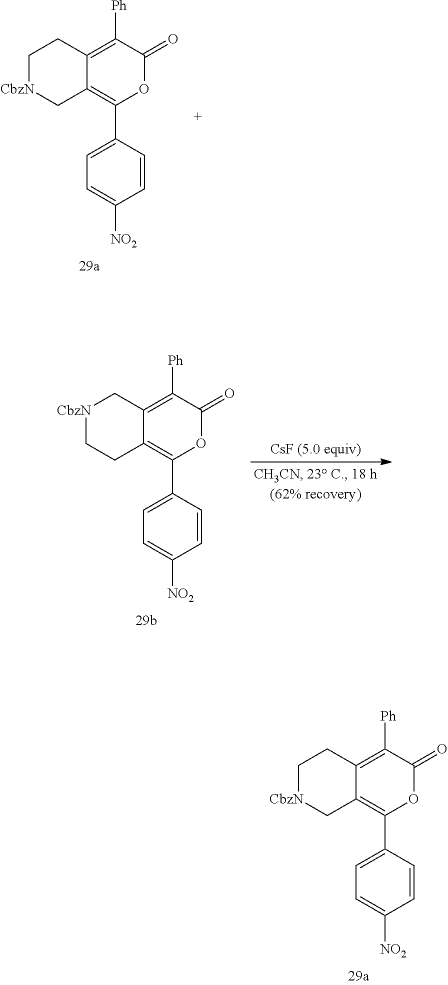 [SMILES: O=C(OCc1ccccc1)N1CCc2c(c(-c3ccc([N+](=O)[O-])cc3)oc(=O)c2-c2ccccc2)C1 ; O=C(OCc1ccccc1)N1CCc2c(-c3ccc([N+](=O)[O-])cc3)oc(=O)c(-c3ccccc3)c2C1 ; O=C(OCc1ccccc1)N1CCc2c(c(-c3ccc([N+](=O)[O-])cc3)oc(=O)c2-c2ccccc2)C1]

Pyrone 29a. Followed General Procedure C using pyrones 29a and 29b (115 mg, 0.236 mmol, 1.5:1 ratio of regioisomers) afforded pyrone 29a (71 mg, 62% recovery) as a yellow foam. Pyrone 29a: R_{f} 0.17 (7:3 hexanes:EtOAc); ¹H-NMR (500 MHz, CD₃CN, 70° C.): δ 8.35-8.28 (m, 2H), 7.86-7.79 (m, 2H), 7.52-7.41 (m, 4H), 7.37-7.30 (m, 6H), 5.07 (s, 2H), 4.54 (s, 2H), 3.57-3.52 (m, 2H), 2.78-2.72 (m, 2H); ¹³C-NMR (125 MHz, CD₃CN, 70° C.): (17 of 22 signals observed) δ 160.7, 150.8, 137.5, 133.7, 129.88, 129.87, 128.4, 128.3, 128.2, 127.9, 127.6, 125.1, 123.8, 66.9, 41.0, 40.7, 27.0; IR (film): 3061, 2948, 1699, 1520, 1342 cm⁻¹; HRMS-APCI (m/z) [M+H]⁺ calcd for C₂₈H₂₃N₂O₆+, 483.1551; found, 483.1546.

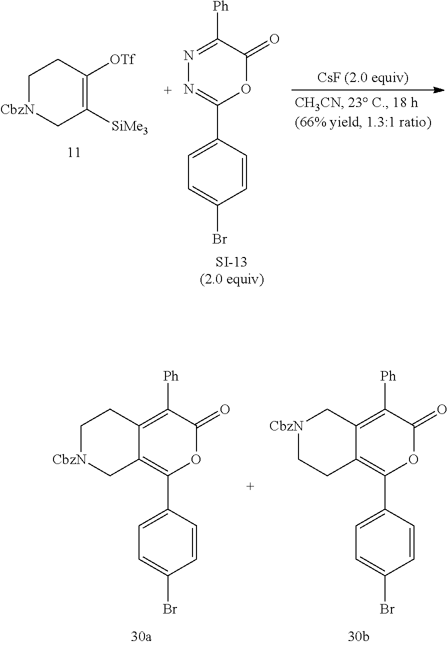 [SMILES: C[Si](C)(C)C1=C(OS(=O)(=O)C(F)(F)F)CCN(C(=O)OCc2ccccc2)C1 ; O=c1oc(-c2ccc(Br)cc2)nnc1-c1ccccc1 ; O=C(OCc1ccccc1)N1CCc2c(c(-c3ccc(Br)cc3)oc(=O)c2-c2ccccc2)C1 ; O=C(OCc1ccccc1)N1CCc2c(-c3ccc(Br)cc3)oc(=O)c(-c3ccccc3)c2C1]

Pyrones 30a and 30b. Followed General Procedure B using silyl triflate 11 (66 mg, 0.15 mmol, 1.0 equiv) afforded pyrones 30a and 30b (66% yield, average of two experiments) as a yellow foam. Pyrones 30a and 30b: R_{f} 0.18 (5:1 hexanes:EtOAc); ¹H-NMR (500 MHz, CD₃CN, 60° C.): δ 7.72-7.69 (m, 3H), 7.56-7.54 (m, 2H), 7.53-7.45 (m, 8H), 7.44-7.40 (m, 3H), 7.36-7.29 (m, 12H), 5.07 (s, 4H), 4.50 (s, 2H, 30a), 4.35 (s, 2H, 30b), 3.56-3.49 (m, 4H), 2.80 (t, J=6.3, 2H, 30b), 2.72 (t, J=6.6, 2H, 30a); HRMS-APCI (m/z) [M+H]⁺ calcd for C₂₈H₂₃BrNO₄⁺, 516.0805; found, 516.0796.

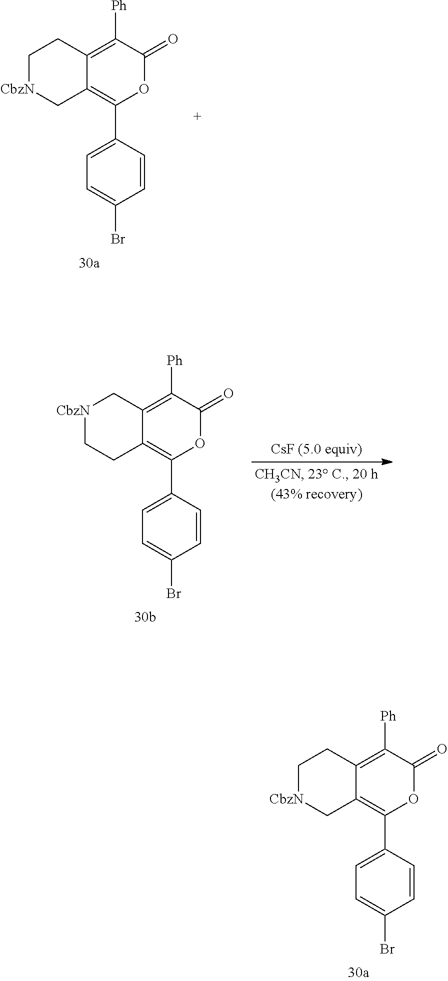 [SMILES: O=C(OCc1ccccc1)N1CCc2c(c(-c3ccc(Br)cc3)oc(=O)c2-c2ccccc2)C1 ; O=C(OCc1ccccc1)N1CCc2c(-c3ccc(Br)cc3)oc(=O)c(-c3ccccc3)c2C1 ; O=C(OCc1ccccc1)N1CCc2c(c(-c3ccc(Br)cc3)oc(=O)c2-c2ccccc2)C1]

Pyrone 30a. Followed General Procedure C using pyrones 30a and 30b (40.0 mg, 0.0775 mmol, 1.3:1 ratio of regioisomers) afforded pyrone 30a (17 mg, 43% recovery) as a yellow foam. Crystals suitable for X-ray diffraction studies were obtained by concentration of pyrone 30a from a mixture of hexanes and EtOAc (CCDC #1876924). Pyrone 30a: mp 71-74° C.; R_{f} 0.18 (5:1 hexanes:EtOAc); ¹H-NMR (500 MHz, CD₃CN, 60° C.): δ 7.72-7.67 (m, 2H), 7.54-7.45 (m, 4H), 7.45-7.40 (m, 1H), 7.38-7.23 (m, 7H), 5.07 (s, 2H), 4.51 (s, 2H), 3.52 (t, J=6.5, 2H), 2.72 (t, J=6.5, 2H); ¹³C-NMR (125 MHz, CD₃CN, 60° C.): (20 of 27 observed) δ 162.3, 154.8, 152.3, 135.1, 134.3, 133.2, 132.1, 131.7, 131.2, 129.6, 129.5, 129.32, 129.27, 129.1, 128.8, 125.4, 113.9, 68.0, 42.3, 41.9; IR (film): 3057, 2924, 2854, 1700, 1417, 1235 cm⁻¹; HRMS-APCI (m/z) [M +H]⁺ calcd for C₂₈H₂₃BrNO₄⁺, 516.0805; found, 516.0772.

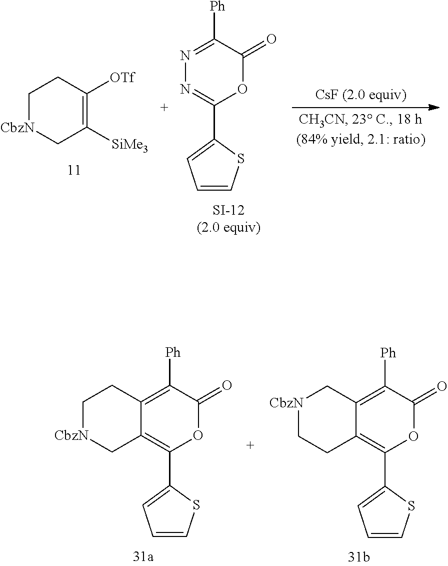 [SMILES: C[Si](C)(C)C1=C(OS(=O)(=O)C(F)(F)F)CCN(C(=O)OCc2ccccc2)C1 ; O=c1oc(-c2cccs2)nnc1-c1ccccc1 ; O=C(OCc1ccccc1)N1CCc2c(c(-c3cccs3)oc(=O)c2-c2ccccc2)C1 ; O=C(OCc1ccccc1)N1CCc2c(-c3cccs3)oc(=O)c(-c3ccccc3)c2C1]

Pyrones 31a and 31b. Followed General Procedure B using silyl triflate 11 (66 mg, 0.150 mmol, 1.0 equiv) afforded pyrones 31a and 31b (84% yield, average of two experiments) as a yellow foam. Pyrones 31a and 31b: R_{f} 0.62 (1:1 hexanes:EtOAc); ¹H-NMR (500 MHz, CD₃CN, 60° C.): 31a: δ 7.72 (dd, J=5.1, 1.1, 1H), 7.56-7.52 (m, 1H), 7.49-7.44 (m, 2H), 7.44-7.39 (m, 1H), 7.38-7.27 (m, 7H), 7.26-7.22 (m, 1H), 5.13 (s, 2H), 4.71 (s, 2H), 3.54 (t, J=6.2, 2H), 2.66 (t, J=6.2, 2H); 31b: δ 7.70 (dd, J=5.1, 1.1, 1H), 7.61 (dd, J=3.8, 1.1, 1H), 7.49-7.44 (m, 2H), 7.44-7.39 (m, 1H), 7.38-7.27 (m, 7H), 7.26-7.22 (m, 1H), 5.07 (s, 2H), 4.32 (s, 2H), 3.65 (t, J=6.2, 2H), 2.95 (t, J=6.2, 2H); HRMS-APCI (m/z) [M+H]⁺ calcd for C₂₆H₂₂NO₄S⁺, 444.1264; found, 444.1254.

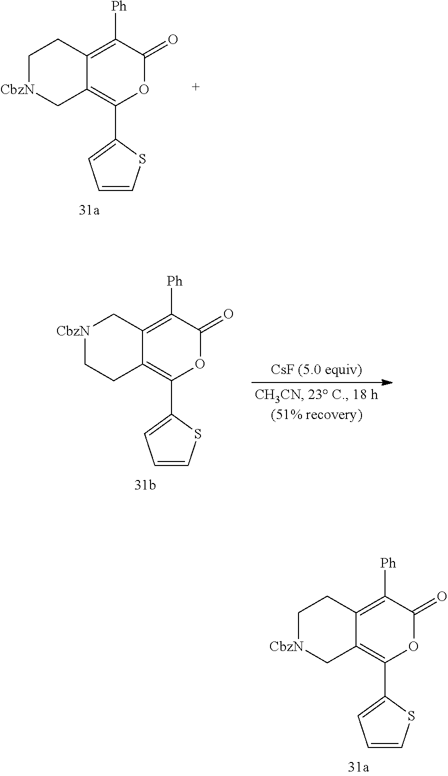 [SMILES: O=C(OCc1ccccc1)N1CCc2c(c(-c3cccs3)oc(=O)c2-c2ccccc2)C1 ; O=C(OCc1ccccc1)N1CCc2c(-c3cccs3)oc(=O)c(-c3ccccc3)c2C1 ; O=C(OCc1ccccc1)N1CCc2c(c(-c3cccs3)oc(=O)c2-c2ccccc2)C1]

Pyrone 31a. Followed General Procedure C using pyrones 31a and 31b (160 mg, 0.361 mmol, 2.1:1 ratio of regioisomers) afforded pyrone 31a (81 mg, 51% recovery) as a yellow foam. Pyrone 31a: R_{f} 0.62 (1:1 hexanes:EtOAc); ¹H-NMR (500 MHz, CD₃CN, 60° C.): δ 7.72 (dd, J=5.0, 0.9, 1H), 7.56-7.52 (m, 1H), 7.49-7.44 (m, 2H), 7.44-7.39 (m, 1H), 7.38-7.29 (m, 7H), 7.26-7.22 (m, 1H), 5.13 (s, 2H), 4.71 (s, 2H), 3.54 (t, J=6.2, 2H), 2.66 (t, J=66.2, 2H); ¹³CNMR (125 MHz, CD₃CN, 60° C.): δ 161.6, 152.2, 150.4, 138.4, 135.2, 135.0, 131.2, 131.0, 130.8, 129.7, 129.5, 129.4, 129.3, 129.1, 128.8, 125.0, 112.4, 68.1, 42.8, 41.8, 28.7; IR (film): 3032, 2925, 2854, 1700, 1418 cm⁻¹; HRMS-APCI (m/z) [M+H]⁺ calcd for C₂₆H₂₂NO₄S⁺, 444.1264; found, 444.1240.

C. Syntheses of Tricyclic Products.

General Procedure D (Preparation of Cycloadduct 18 is Used as an Example of Embodiments).

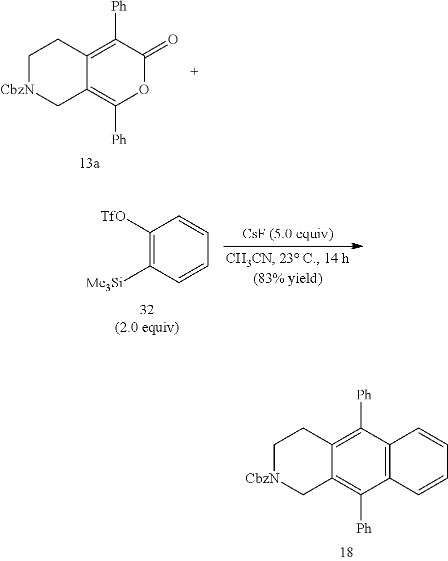 [SMILES: O=C(OCc1ccccc1)N1CCc2c(c(-c3ccccc3)oc(=O)c2-c2ccccc2)C1 ; C[Si](C)(C)c1ccccc1OS(=O)(=O)C(F)(F)F ; O=C(OCc1ccccc1)N1CCc2c(c(-c3ccccc3)c3ccccc3c2-c2ccccc2)C1]

Cycloadduct 18. To a stirred solution of pyrone 13a (44 mg, 0.10 mmol, 1.0 equiv) and silyl triflate 32 (60 mg, 0.20 mmol, 2.0 equiv) in acetonitrile (1.0 mL) was added CsF (76 mg, 0.50 mmol, 5.0 equiv) in one portion. The reaction was purged with nitrogen for ten minutes before being sealed with a Teflon cap and left to stir at 23° C. After 14 h, the reaction mixture was filtered through celite (monster pipette, ˜4 cm tall) with EtOAc (˜10 mL) as the eluent and then concentrated under reduced pressure. Purification of the crude material via flash chromatography (7:3 hexanes:EtOAc) afforded cycloadduct 18 (83% yield, average of two experiments) as a light yellow foam. Cycloadduct 18: R_{f} 0.57 (5:1 hexanes:EtOAc); ¹H-NMR (500 MHz, CD₃CN, 60° C.): δ 7.61-7.50 (m, 6H), 7.41-7.30 (m, 12H), 5.05 (s, 2H), 4.48 (s, 2H), 3.59 (t, J=6.3, 2H), 2.77 (t, J=6.3, 2H); ¹³C-NMR (125 MHz, CD₃CN, 60° C.): δ 140.2, 139.5, 138.9, 138.6, 137.5, 132.9, 132.8, 132.6, 131.5, 131.4, 131.2, 130.0, 129.8, 129.6, 129.0, 128.9, 128.7, 127.2, 127.1, 126.8, 126.6, 67.7, 45.9, 43.1, 28.8; IR (film): 3062, 2935, 2887, 1698, 1233 cm⁻¹; HRMS-APCI (m/z) [M+Na]⁺ calcd for C₃₃H₂₇NO₂Na⁺, 492.1934; found, 492.1898.

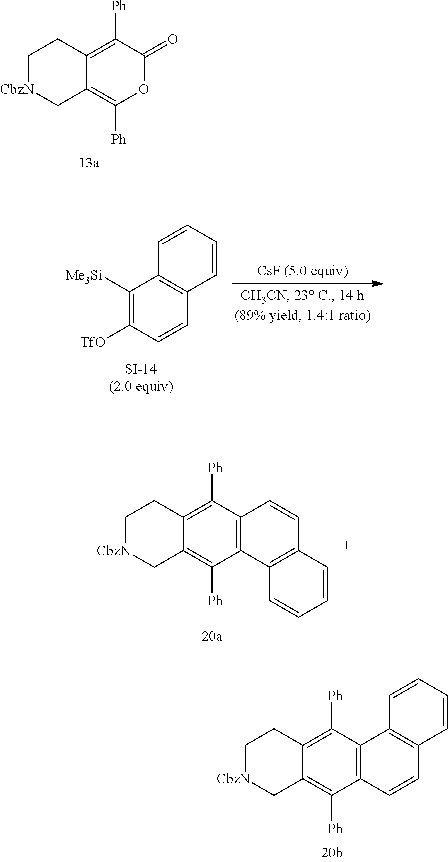 [SMILES: O=C(OCc1ccccc1)N1CCc2c(c(-c3ccccc3)oc(=O)c2-c2ccccc2)C1 ; C[Si](C)(C)c1c(OS(=O)(=O)C(F)(F)F)ccc2ccccc12 ; O=C(OCc1ccccc1)N1CCc2c(c(-c3ccccc3)c3c(ccc4ccccc43)c2-c2ccccc2)C1 ; O=C(OCc1ccccc1)N1CCc2c(c(-c3ccccc3)c3ccc4ccccc4c3c2-c2ccccc2)C1]

Cycloadducts 20a and 20b. Followed General Procedure D using pyrone 13a (44 mg, 0.100 mmol, 1.0 equiv) afforded, after purification via flash chromatography (Biotage 10 g SiO₂, 19:1→2:3 hexanes:EtOAc), cycloadducts 20a and 20b (89% yield, 1.4:1 ratio of regioisomers, average of two experiments) as a yellow foam. Cycloadducts 20a and 20b: R_{f} 0.37 (5:2 hexanes:EtOAc); ¹H-NMR (500 MHz, CD₃CN, 60° C.): δ 7.79 (dd, J=7.5, 1.6, 2H), 7.63-7.48 (m, 18H), 7.42-7.38 (m, 2H), 7.37-7.28 (m, 18H), 7.05-7.00 (m, 2H), 5.08-5.03 (m, 4H), 4.45 (s, 4H), 3.63-3.56 (m, 4H), 2.75-2.69 (m, 4H); ¹³C-NMR (125 MHz, CD₃CN): δ (52 of 62 signals observed) 156.3, 144.3, 143.6, 140.8, 140.4, 140.1, 139.7, 138.7, 138.6, 134.8, 134.6, 134.5, 133.6, 133.5, 132.23, 132.16, 131.8, 131.7, 131.4, 131.2, 131.00, 130.95, 130.83, 130.80, 130.1, 130.0, 129.7, 129.64, 129.60, 129.3, 129.1, 129.01, 128.97, 128.9, 128.70, 128.68, 128.5, 128.3, 128.2, 127.2, 127.1, 126.3, 126.19, 126.18, 126.0, 67.7, 46.6, 46.2, 43.0, 42.7, 29.4, 29.0; IR (film): 3055, 2939, 2889, 1698, 1235 cm⁻¹; HRMS-APCI (m/z) [M+H]⁺ calcd for C₃₇H₃₀NO₂⁺, 520.2271; found, 520.2242. Note: All peaks in the ¹H-NMR spectrum of 20a and 20b were overlapping. However, the ratio of the two compounds was determined via deconvolution of the peaks at 5.06 and 5.04 ppm.

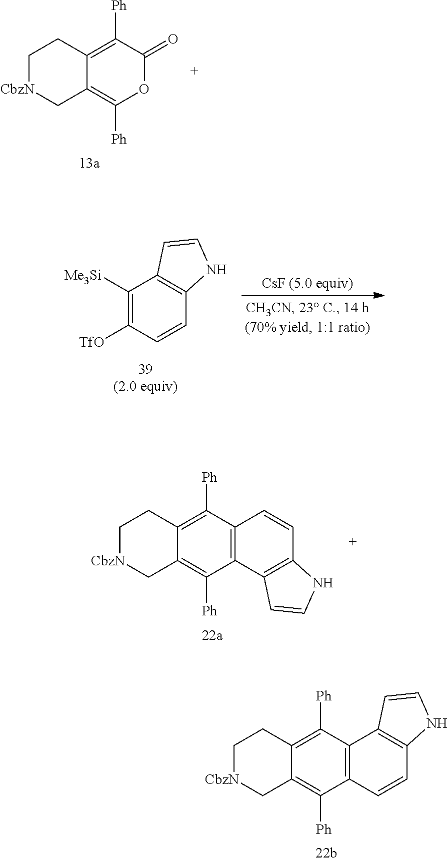 [SMILES: O=C(OCc1ccccc1)N1CCc2c(c(-c3ccccc3)oc(=O)c2-c2ccccc2)C1 ; C[Si](C)(C)c1c(OS(=O)(=O)C(F)(F)F)ccc2[nH]ccc12 ; O=C(OCc1ccccc1)N1CCc2c(c(-c3ccccc3)c3c(ccc4[nH]ccc43)c2-c2ccccc2)C1 ; O=C(OCc1ccccc1)N1CCc2c(c(-c3ccccc3)c3ccc4[nH]ccc4c3c2-c2ccccc2)C1]

Cycloadducts 22a and 22b. Followed General Procedure D using pyrone 13a (44 mg, 0.100 mmol, 1.0 equiv) afforded, after purification via flash chromatography (Isco 4 g gold SiO₂, 100% hexanes→100% EtOAc), cycloadducts 22a and 22b (70% yield, average of two experiments) as a yellow foam. Cycloadducts 22a and 22b: R_{f} 0.38 (5:2 hexanes:EtOAc); ¹H-NMR (500 MHz, CD₃CN, 60° C.): δ 9.44 (br s, 2H), 7.63-7.41 (m, 15H), 7.38-7.21 (m, 17H), 7.11 (dd, J=9.0, 6.1, 2H), 6.91 (dd, J=2.9, 2.9, 2H), 5.08-5.02 (m, 6H), 4.46 (s, 2H), 4.44 (s, 2H), 3.60 (t, J=6.1, 4H), 2.75-2.68 (m, 4H); ¹³C-NMR (125 MHz, CD₃CN, 60° C.): (47 of 58 signals observed) δ 156.4, 143.4, 142.6, 141.7, 141.0, 139.9, 138.6, 138.3, 137.8, 134.6, 134.4, 132.5, 131.4, 131.23, 131.19, 131.2, 131.1, 130.5, 130.3, 129.9, 129.8, 129.6, 129.3, 129.0, 128.94, 128.89, 128.63, 128.62, 128.58, 128.4, 128.2, 127.5, 127.1, 122.9, 122.74, 122.68, 122.0, 121.9, 114.4, 114.3, 105.9, 105.8, 67.7, 46.2, 46.1, 43.12, 43.08; IR (film): 3321, 3054, 2937, 1681, 1239 cm⁻¹; HRMS-APCI (m/z) [M−Cbz+H]⁺ calcd for C₂₇H₂₃N₂⁺, 375.1856; found, 375.1834. Note: All peaks in the ¹H-NMR spectrum of 22a and 22b were overlapping. However, the ratio of the two compounds was determined via deconvolution of the peaks at 4.46 and 4.44 ppm.

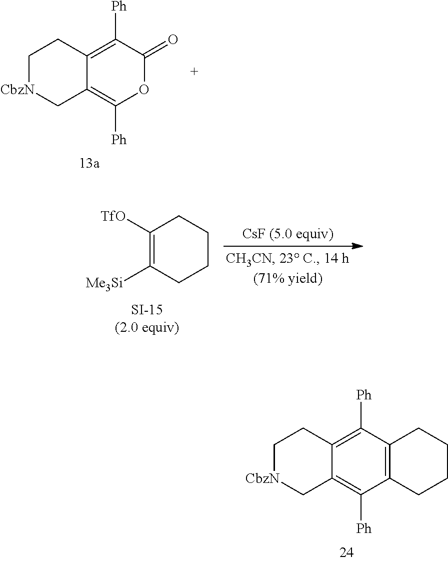 [SMILES: O=C(OCc1ccccc1)N1CCc2c(c(-c3ccccc3)oc(=O)c2-c2ccccc2)C1 ; C[Si](C)(C)C1=C(OS(=O)(=O)C(F)(F)F)CCCC1 ; O=C(OCc1ccccc1)N1CCc2c(c(-c3ccccc3)c3c(c2-c2ccccc2)CCCC3)C1]

Cycloadduct 24. Followed General Procedure D using pyrone 13a (44 mg, 0.100 mmol, 1.0 equiv) afforded, after purification via flash chromatography (Biotage 10 g SiO₂, 19:1→2:3 hexanes:EtOAc), cycloadduct 24 (71% yield, average of two experiments) as a yellow foam. Cycloadduct 24: R_{f} 0.54 (5:2 hexanes:EtOAc); ¹H-NMR (500 MHz, CD₃CN, 60° C.): δ 7.51-7.45 (m, 4H), 7.44-7.29 (m, 6H), 7.28-7.24 (m, 1H), 7.17-7.13 (m, 4H), 5.03 (s, 2H), 4.16 (s, 2H), 3.49 (t, J=6.0, 2H), 2.42 (t, J=6.0, 2H), 2.36-2.31 (m, 4H), 1.64-1.59 (m, 4H); ¹³C-NMR (125 MHz, CD₃CN, 60° C.): (24 of 27 signals observed) δ 156.3, 141.9, 141.5, 140.8, 134.8, 134.4, 131.3, 130.4, 130.24, 130.17, 130.0, 129.8, 129.6, 128.9, 128.3, 128.0, 67.6, 46.0, 42.7, 31.0, 29.7, 29.4, 23.88, 23.85; IR (film): 3054, 2932, 2857, 1702, 1434 cm⁻¹; HRMS-APCI (m/z) [M+H]⁺ calcd for C₃₃H₃₂NO₂⁺, 474.2428; found, 474.2318.

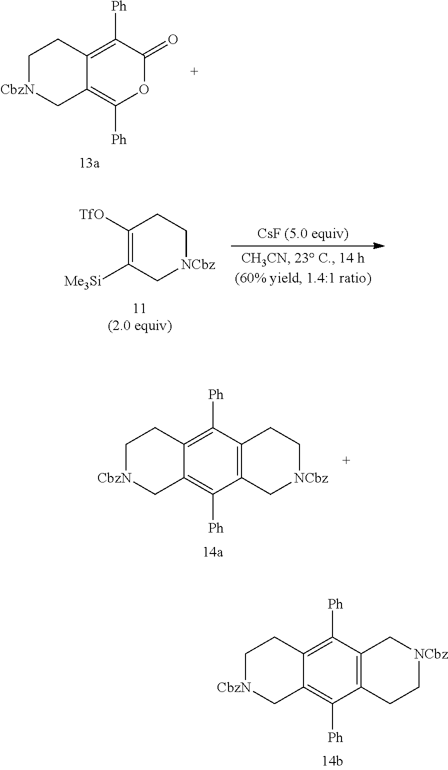 [SMILES: O=C(OCc1ccccc1)N1CCc2c(c(-c3ccccc3)oc(=O)c2-c2ccccc2)C1 ; C[Si](C)(C)C1=C(OS(=O)(=O)C(F)(F)F)CCN(C(=O)OCc2ccccc2)C1 ; O=C(OCc1ccccc1)N1CCc2c(c(-c3ccccc3)c3c(c2-c2ccccc2)CCN(C(=O)OCc2ccccc2)C3)C1 ; O=C(OCc1ccccc1)N1CCc2c(c(-c3ccccc3)c3c(c2-c2ccccc2)CN(C(=O)OCc2ccccc2)CC3)C1]

Cycloadducts 14a and 14b. Followed General Procedure D using pyrone 13a (44 mg, 0.100 mmol, 1.0 equiv) afforded, after purification via flash chromatography (Biotage 10 g SiO₂, 19:1→2:3 hexanes:EtOAc), cycloadducts 14a and 14b (60% yield, 1.4:1 ratio of regioisomers, average of two experiments) as a yellow foam. Cycloadducts 14a and 14b: R_{f} 0.27 (5:2 hexanes:EtOAc); ¹H-NMR (500 MHz, CD₃CN, 70° C.): δ 7.54-7.41 (m, 12H), 7.38-7.22 (m, 20H), 7.21-7.14 (m, 8H) 5.04 (s, 8H), 4.23 (s, 4H), 4.21 (s, 4H), 3.51 (t, J=6.0, 8H), 2.52-2.44 (m, 8H); ¹³C-NMR (125 MHz, CD₃CN, 70° C.): (31 of 37 signals observed) δ 156.4, 141.8, 140.6, 140.0, 139.9, 139.2, 138.7, 138.3, 132.9, 132.5, 131.7, 131.2, 130.6, 130.4, 130.3, 130.2, 130.1, 129.9, 129.6, 129.1, 129.01, 128.96, 128.70, 128.68, 128.5, 67.7, 46.1, 45.9, 42.7, 28.6, 28.3; IR (film): 3057, 3030, 2937, 1697, 1232 cm⁻¹; HRMS-APCI (m/z) [M−Cbz]^{⋅-} calcd for C₃₂H₂₉N₂O₂^{⋅-}, 473.2224; found, 473.2201. Note: All peaks in the ¹H-NMR spectrum of 14a and 14b were overlapping. However, the ratio of the two compounds was determined via deconvolution of the peaks at 4.23 and 4.21 ppm.

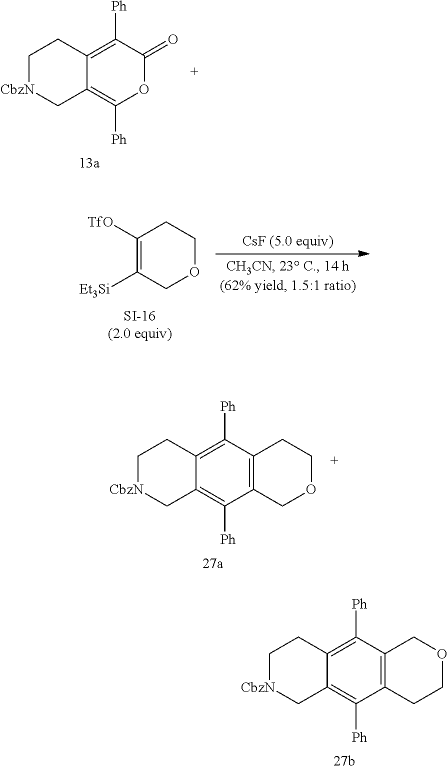

Cycloadduct 27a and 27b. Followed General Procedure D using pyrone 13a (28 mg, 0.065 mmol, 1.0 equiv) afforded, after purification via preparative thin layer chromatography (5:2 hexanes:EtOAc), cycloadducts 27a and 27b (62% yield, average of two experiments) as a yellow foam. Cycloadducts 27a and 27b: R_{f} 0.57 (9:1 hexanes:EtOAc); ¹H-NMR (500 MHz, CD₃CN, 60° C.): δ 7.53-7.46 (m, 8H), 7.46-7.39 (m, 4H), 7.36-7.29 (m, 6H), 7.28-7.22 (m, 3H), 7.21-7.15 (m 8H), 5.03 (s, 4H), 4.31 (s, 2H), 4.30 (s, 2H), 4.21 (s, 2H), 4.20 (s, 2H), 3.77-3.73 (m, 4H), 3.51 (t, J=6.1, 4H), 2.49-2.43 (m, 4H), 2.42-2.37 (m, 4H); ¹³C-NMR (125 MHz, CD₃CN, 70° C.): (44 of 52 signals observed) δ 156.4, 142.0, 140.6, 140.2, 139.9, 139.5, 139.0, 138.8, 138.7, 137.2, 132.8, 132.6, 132.3, 132.1, 131.5, 131.4, 131.0, 130.9, 130.5, 130.4, 130.3, 130.2, 130.14, 130.12, 130.0, 129.9, 129.6, 129.0, 128.9, 128.7, 128.63, 128.62, 128.4, 68.2, 68.0, 67.7, 65.89, 65.88, 46.0, 45.7, 42.70, 42.68, 28.7, 28.5; IR (film): 3057, 2954, 2850, 1698, 1231 cm⁻¹; HRMS-APCI (m/z) [M+K]⁺ calcd for C₃₂H₂₉NO₃K⁺, 514.1779; found, 514.1752. Note: All peaks in the ¹H-NMR spectrum of 27a and 27b were overlapping. However, the ratio of the two compounds was determined via deconvolution of the peaks at 4.22 and 4.20 ppm.

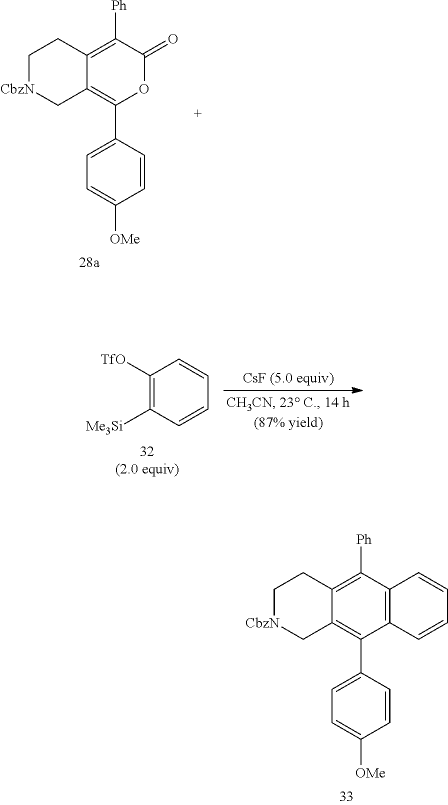 [SMILES: COc1ccc(-c2oc(=O)c(-c3ccccc3)c3c2CN(C(=O)OCc2ccccc2)CC3)cc1 ; C[Si](C)(C)c1ccccc1OS(=O)(=O)C(F)(F)F ; COc1ccc(-c2c3c(c(-c4ccccc4)c4ccccc24)CCN(C(=O)OCc2ccccc2)C3)cc1]

Cycloadduct 33. Followed General Procedure D using pyrone 28a (21 mg, 0.045 mmol, 1.0 equiv) afforded, after purification via preparative thin layer chromatography (4:1 hexanes:EtOAc), cycloadduct 33 (87% yield, average of two experiments) as a yellow foam. Cycloadduct 33: R_{f} 0.41 (4:1 hexanes:EtOAc); ¹H-NMR (500 MHz, CD₃CN, 60° C.): δ 7.60-7.55 (m, 2H), 7.53-7.49 (m, 1H), 7.46-7.42 (m, 1H), 7.40-7.26 (m, 10H), 7.25-7.21 (m, 2H), 7.15-7.09 (m, 2H), 5.06 (s, 2H), 4.50 (s, 2H), 3.92 (s, 3H), 3.58 (t, J=6.2, 2H), 2.76 (t, J=6.2, 2H); ¹³C-NMR (125 MHz, CD₃CN, 60° C.): (25 of 28 signals observed) δ 160.6, 156.3, 140.2, 138.7, 138.6, 137.2, 132.9, 132.8, 132.4, 131.9, 131.4, 129.8, 129.6, 129.0, 128.7, 128.6, 127.2, 127.1, 126.7, 126.5, 115.5, 67.7, 56.3, 45.9, 43.1; IR (film): 3063, 3031, 2934, 1697, 1243 cm⁻¹; HRMS-APCI (m/z) [M+K]⁺ calcd for C₃₄H₂₉NO₃K⁺, 538.1779; found, 538.1752.

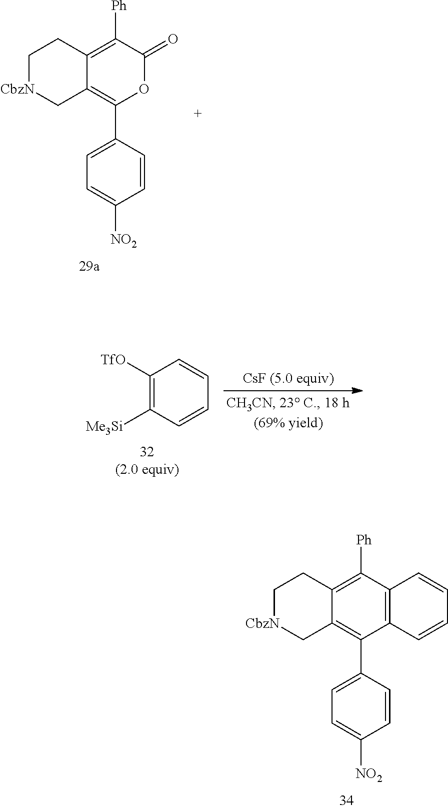 [SMILES: O=C(OCc1ccccc1)N1CCc2c(c(-c3ccc([N+](=O)[O-])cc3)oc(=O)c2-c2ccccc2)C1 ; C[Si](C)(C)c1ccccc1OS(=O)(=O)C(F)(F)F ; O=C(OCc1ccccc1)N1CCc2c(c(-c3ccc([N+](=O)[O-])cc3)c3ccccc3c2-c2ccccc2)C1]

Cycloadduct 34. Followed General Procedure D using pyrone 29a (27 mg, 0.056 mmol, 1.0 equiv) afforded, after purification via flash chromatography (Isco 4 g gold SiO₂, 1:1 hexanes:EtOAc), cycloadduct 34 (69% yield, average of two experiments) as a yellow foam. Cycloadduct 34: R_{f} 0.63 (7:3 hexanes:EtOAc); ¹H-NMR (500 MHz, CD₃CN, 70° C.): δ 8.41-8.33 (m, 2H), 7.62-7.50 (m, 5H), 7.46-7.41 (m, 1H), 7.40-7.25 (m, 10H), 5.05 (s, 2H), 4.46 (s, 2H), 3.60 (t, J=6.1, 2H), 2.79 (t, J=6.1, 2H); ¹³C-NMR (125 MHz, CD₃CN, 70° C.): (22 of 27 observed) δ 146.8, 140.0, 139.9, 133.04, 132.97, 132.8, 131.9, 131.8, 131.4, 129.9, 129.6, 129.1, 128.82, 128.80, 127.5, 127.1, 126.7, 125.1, 67.9, 45.7, 43.1, 28.7; IR (film): 3062, 2839, 2862, 1699, 1518, 1348 cm⁻¹; HRMS-APCI (m/z) [M−Cbz]⁻ calcd for C₂₅H₁₉N₂O₂⁻, 379.1441; found, 379.1432.

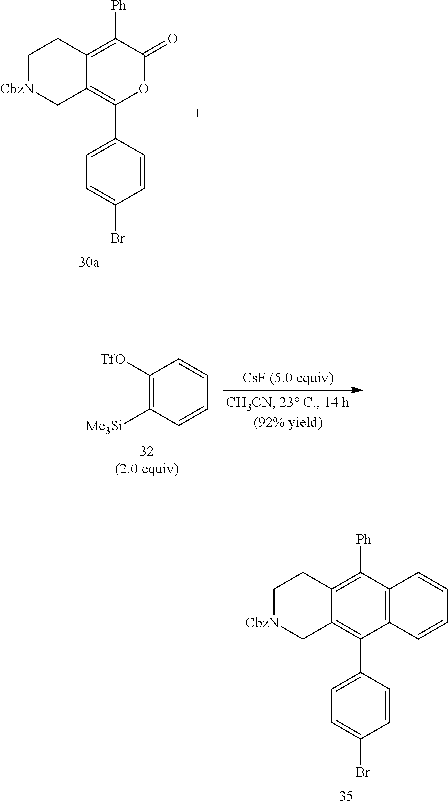 [SMILES: O=C(OCc1ccccc1)N1CCc2c(c(-c3ccc(Br)cc3)oc(=O)c2-c2ccccc2)C1 ; C[Si](C)(C)c1ccccc1OS(=O)(=O)C(F)(F)F ; O=C(OCc1ccccc1)N1CCc2c(c(-c3ccc(Br)cc3)c3ccccc3c2-c2ccccc2)C1]

Cycloadduct 35. Followed General Procedure D using pyrone 30a (21 mg, 0.041 mmol, 1.0 equiv) afforded, after purification via flash chromatography (Biotage 10 g SiO₂, 5:1 hexanes:EtOAc), cycloadduct 35 (92% yield, average of two experiments) as a yellow foam. Cycloadduct 35: R_{f} 0.50 (4:1 hexanes:EtOAc); ¹H-NMR (500 MHz, CD₃CN, 60° C.): δ 7.76-7.71 (m, 2H), 7.60-7.51 (m, 3H), 7.42-7.23 (m, 13H), 5.05 (s, 2H), 4.47 (s, 2H), 3.62-3.57 (m, 2H), 2.79-2.74 (m, 2H); ¹³C-NMR (125 MHz, CD₃CN, 60° C.): δ 156.3, 140.0, 139.3, 138.6, 138.5, 136.0, 133.3, 133.1, 132.9, 132.8, 132.3, 131.7, 131.6, 131.3, 129.8, 129.6, 129.0, 128.7, 127.3, 126.91, 126.90, 126.8, 122.6, 67.8, 45.7, 43.1, 28.7; IR (film): 3062, 3032, 2929, 1700, 1215 cm⁻¹; HRMS-APCI (m/z) [M−Cbz]⁻ calcd for C₂₅H₁₉BrN⁻, 412.0695; found, 412.0669.

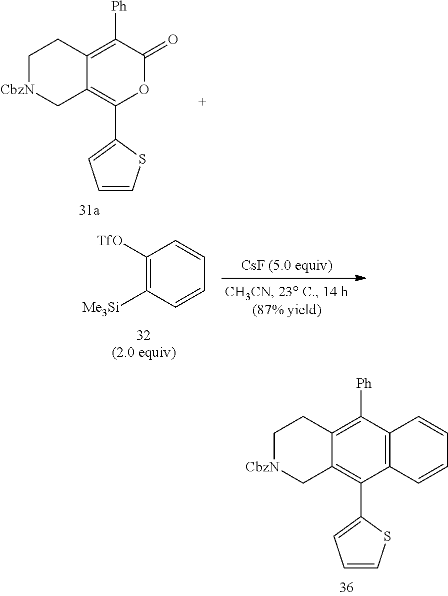 [SMILES: O=C(OCc1ccccc1)N1CCc2c(c(-c3cccs3)oc(=O)c2-c2ccccc2)C1 ; C[Si](C)(C)c1ccccc1OS(=O)(=O)C(F)(F)F ; O=C(OCc1ccccc1)N1CCc2c(c(-c3cccs3)c3ccccc3c2-c2ccccc2)C1]

Cycloadduct 36. Followed General Procedure D using pyrone 31a (38 mg, 0.086 mmol, 1.0 equiv) afforded, after purification via flash chromatography (Isco gold 4 g SiO₂, 1:1 hexanes:EtOAc), cycloadduct 36 (87% yield, average of two experiments) as a yellow foam. Cycloadduct 36: R_{f} 0.78 (7:3 hexanes:EtOAc); ¹H-NMR (500 MHz, CD₃CN, 60° C.): δ 7.68-7.66 (m, 1H), 7.61-7.55 (m, 3H), 7.53-7.49 (m, 1H), 7.42-7.33 (m, 5H), 7.32-7.29 (m, 2H), 7.10-7.07 (m, 1H), 5.08 (s, 2H), 4.62 (s, 2H), 3.59 (t, J=6.2, 2H), 2.74 (t, J=6.2, 2H); ¹³CNMR (125 MHz, CD₃CN, 60° C.): δ 156.3, 140.2, 139.9, 139.1, 138.5, 134.2, 133.7, 132.8, 132.7, 131.2, 129.9, 129.8, 129.6, 129.5, 129.0, 128.8, 128.72, 128.69, 128.2, 127.2, 127.00, 126.98, 126.8, 67.7, 45.9, 43.0, 28.8; IR (film): 3064, 2939, 2888, 1697, 1420 cm⁻¹; HRMS-APCI (m/z) [M−Cbz]⁻ calcd for C₂₃H₁₈NS⁻, 340.1155; found, 340.1133.

D. One-Pot Three-Component Coupling.

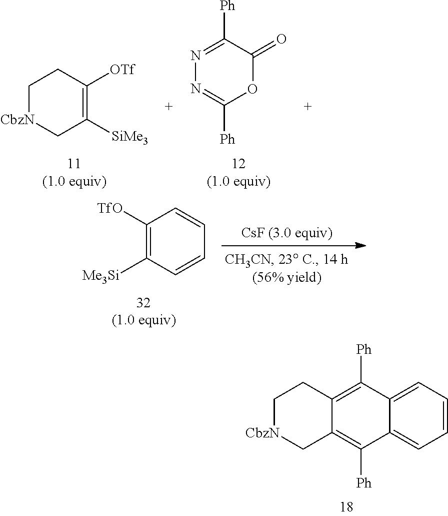 [SMILES: C[Si](C)(C)C1=C(OS(=O)(=O)C(F)(F)F)CCN(C(=O)OCc2ccccc2)C1 ; O=c1oc(-c2ccccc2)nnc1-c1ccccc1 ; C[Si](C)(C)c1ccccc1OS(=O)(=O)C(F)(F)F ; O=C(OCc1ccccc1)N1CCc2c(c(-c3ccccc3)c3ccccc3c2-c2ccccc2)C1]

Cycloadduct 18. A solution of silyl triflate 11 (25.0 mg, 0.057 mmol, 1.0 equiv), oxadiazinone 12 (14.3 mg, 0.057 mmol, 1.0 equiv), and silyl triflate 32 (17.0 mg, 0.057 mmol, 1.0 equiv) in acetonitrile (5.7 mL) was purged with nitrogen for 10 min. Then, CsF (26.0 mg, 0.171 mmol, 3.0 equiv) was added and the reaction was allowed to stir at 23° C. for 14 h. Upon completion of the reaction, it was filtered through celite (monster pipette, ˜4 cm tall) using CH₂Cl₂ (˜10 mL) as the eluent and concentrated under reduced pressure. The crude residue was purified using preparative thin layer chromatography (100% benzene) to afford cycloadduct 18 (15.0 mg, 56% yield) as a yellow oil. Spectral data matched those already reported herein for 18.

E. Synthesis of Four Coordinate Products.

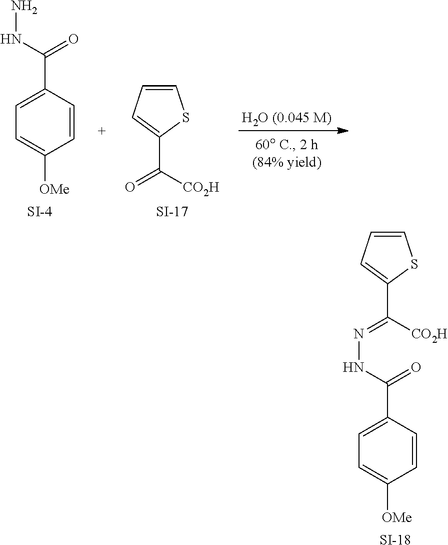

Hydrazone SI-18. Followed General Procedure A using hydrazide SI-4 (0.500 g, 3.20 mmol) to afford hydrazone SI-18 (0.820 g, 84% yield) as a tan solid. Hydrazone SI-18: mp 172-173° C.; R_{f} 0.26 (9:1 EtOAc:MeOH); ¹H-NMR (500 MHz, DMSO-d₆): δ 12.88 (br s, 1H), 7.86-7.82 (m, 2H), 7.67 (dd, J=3.6, 1.0, 1H), 7.63 (dd, J=5.0, 1.1, 1H), 7.13-7.08 (m, 3H), 3.85 (s, 3H); ¹³CNMR (125 MHz, DMSO-d₆): (9 of 12 signals observed) δ 163.1, 162.5, 139.0, 129.4, 128.9, 127.8, 124.7, 114.2, 55.5; IR (film): 3210, 3012, 2838, 1691, 1483 cm⁻¹; HRMS-APCI (m/z) [M+H]⁺ calcd for C₁₄H₁₃N₂O₄S⁺, 305.0591; found, 305.0595.

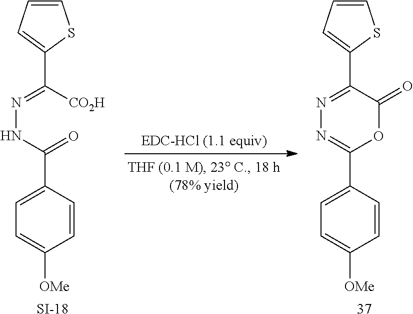

Oxadiazinone 37. Followed General Procedure A using hydrazone SI-18 (0.750 g, 3.20 mmol) to afford oxadiazinone 37 (0.550 g, 78% yield) as a yellow solid after recrystallization from hot EtOAc. Oxadiazinone 37: mp 146-147° C.; R_{f} 0.67 (5:2 hexanes:EtOAc); ¹H-NMR (500 MHz, CDCl₃): δ 8.31 (dd, J=3.8, 1.1, 1H), 8.22-8.18 (m, 2H), 7.61 (dd, J=5.1, 1.1, 1H), 7.19 (dd, J=5.1, 3.8, 1H), 7.04-7.00 (m, 2H), 3.90 (s, 3H); ¹³C-NMR (125 MHz, CDCl₃): δ 164.2, 156.9, 147.9, 147.8, 135.0, 133.3, 132.5, 130.3, 128.8, 119.9, 114.8, 55.7; IR (film): 3123, 3076, 2847, 1750, 1603 cm⁻¹; HRMS-APCI (m/z) [M+H]⁺ calcd for C₁₄H₁₁N₂O₃S⁺, 287.0485; found, 287.0470.

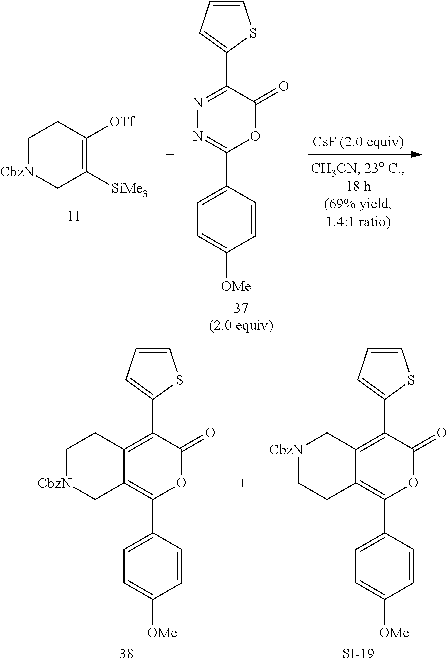

Pyrones 38 and SI-19. Followed General Procedure B using silyl triflate 11 (680 mg, 1.55 mmol, 1.0 equiv) afforded pyrones 38 and SI-19 (69% yield, 1.4:1 ratio of regioisomers) as a yellow oil. Pyrones 38 and SI-19: R_{f} 0.24 (7:3 hexanes:EtOAc); ¹H-NMR (500 MHz, CD₃CN, 60° C.): δ 7.61-7.52 (m, 5H), 7.50-7.38 (m, 10H), 7.37-7.27 (m, 16H), 7.09-7.04 (m, 5H), 5.08-5.04 (m, 4H), 4.54 (s, 2H), 4.34 (s, 2H), 3.88 (s, 3H), 3.87 (s, 3H), 3.56-3.48 (m, 4H), 2.83 (t, J=6.4, 2H), 2.71 (t, J=6.4, 2H); HRMS-APCI (m/z) [M+H]⁺ calcd for C₂₇H₂4NO₅S⁺, 474.1370; found, 474.1362.

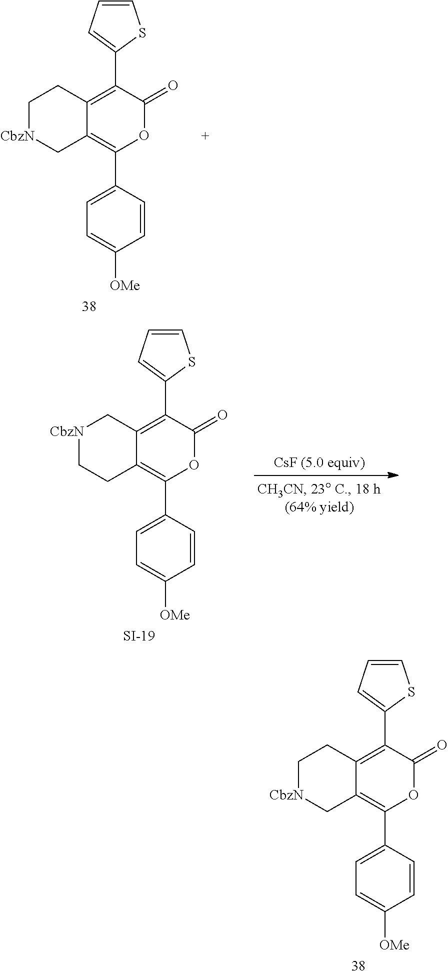

Pyrone 38. Followed General Procedure C using pyrones 38 and SI-19 (85 mg, 0.179 mmol, 1.0 equiv) afforded pyrone 38 (64% yield) as a yellow oil. Pyrone 38: R_{f} 0.24 (7:3 hexanes:EtOAc); ¹H-NMR (500 MHz, CD₃CN, 60° C.): δ 7.57-7.52 (m, 3H), 7.36-7.24 (m, 5H), 7.17 (dd, J=3.6, 1.3, 1H), 7.16-7.13 (m, 1H), 7.09-7.04 (m, 2H), 5.07 (s, 2H), 4.53 (s, 2H), 3.88 (s, 3H), 3.54 (t, J=6.5, 2H), 2.95 (t, J=6.5, 2H); ¹³C-NMR (125 MHz, CD₃CN, 60° C.): (21 of 23 observed) δ 162.7, 162.1, 156.4, 153.9, 138.4, 135.4, 131.5, 130.6, 129.6, 129.1, 128.8, 128.6, 127.8, 125.1, 117.6, 115.5, 112.9, 68.0, 56.5, 42.14, 42.10, 28.7; IR (film): 3072, 2937, 1699, 1507, 1257 cm⁻¹; HRMS-APCI (m/z) [M+H]⁺ calcd for C₂₇H₂₄NO₅S⁺, 474.1370; found, 474.1334. Note: Pyrone 38 could be directly accessed in one step from silyl triflate 11 and oxadiazinone 37 using 5.0 equiv of CsF. This reaction results in a 41% yield (as previously shown herein) of pyrone 38 as a single regioisomer.

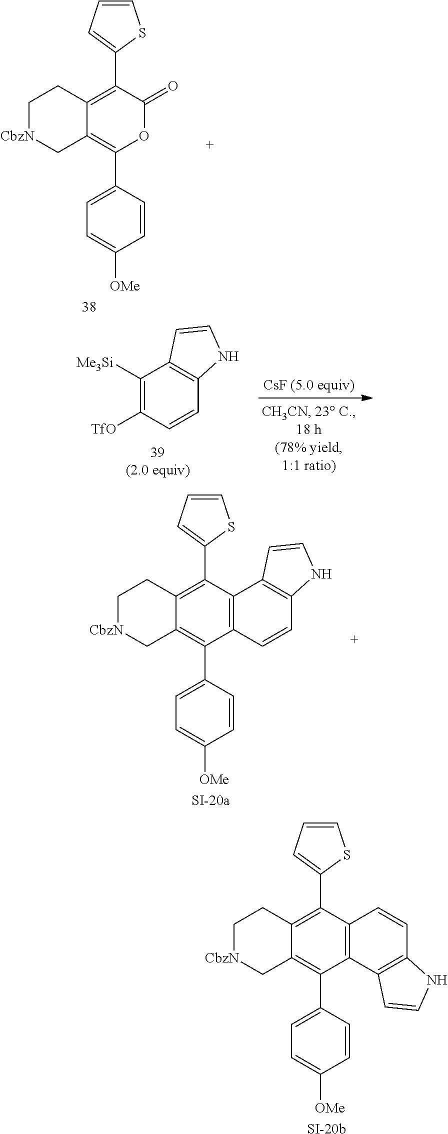

Cycloadducts SI-20a and SI-20b. Followed General Procedure D using pyrone 38 (200 mg, 0.422 mmol, 1.0 equiv) afforded, after purification via flash chromatography (7:3 hexanes:EtOAc), cycloadducts SI-20a and SI-20b (78% yield, 1:1 ratio of regioisomers) as a yellow oil. Cycloadducts SI-20a and SI-20b: R_{f} 0.25 (3:1 hexanes:EtOAc); ¹H-NMR (500 MHz, CD₃CN, 60° C.): δ 9.48 (br s, 2H), 7.69 (dd, J=5.2, 1.2, 1H), 7.63 (dd, J=5.2, 1.2, 1H), 7.52 (dd, J=9.2, 0.8, 1H), 7.48 (dd, J=9.1, 1.0, 1H), 7.37-7.25 (m. 12H), 7.23-7.19 (m, 4H), 7.17-7.08 (m, 5H), 7.06-7.04 (m, 2H), 7.03-7.01 (m, 1H), 6.96-6.94 (m, 1H), 5.31-5.29 (m, 1H), 5.16-5.13 (m, 1H), 5.08-5.04 (m, 4H), 5.48-5.42 (m, 4H), 3.94 (s, 3H), 3.91 (s, 3H), 3.65-3.60 (m, 4H), 2.92-2.80 (m, 4H); ¹³C-NMR (125 MHz, CD₃CN, 60° C.): (57 of 60 observed) δ 159.7, 159.3, 155.1, 142.5, 140.5, 138.2, 134.1, 133.6, 133.3, 133.2, 131.4, 131.00, 130.97, 130.71, 130.70, 130.6, 130.4, 130.3, 129.1, 128.6, 128.37, 128.36, 128.2, 128.1, 127.86, 127.85, 127.8, 127.73, 127.69, 127.54, 127.53, 127.44, 127.44, 127.43, 127.36, 126.6, 126.4, 126.3, 121.9, 121.7, 121.3, 120.8, 120.4, 114.8, 114.2, 113.6, 113.1, 104.7, 104.3, 66.46, 66.45, 55.2, 55.1, 44.9, 44.8, 41.8, 41.7; IR (film): 3416, 3032, 2931, 1678, 1244 cm⁻¹; HRMS-APCI (m/z) [M+H]⁺ calcd for C₃₄H₂₉N₂O₃S⁺, 545.1893; found, 545.1887.

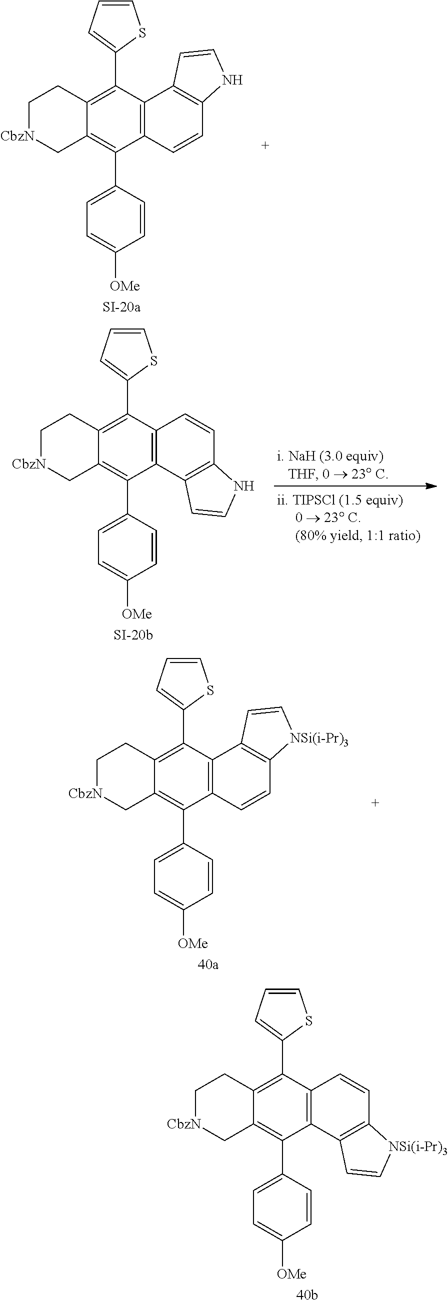

Cycloadducts 40a and 40b. To a solution of NaH (33.0 mg, 60% w/w dispersion in mineral oil, 0.826 mmol, 3.0 equiv) in THF (5.0 mL) at 0° C., was cannula transferred a 0° C. solution of cycloadducts SI-20a and SI-20b (150 mg, 0.275 mmol, 1.0 equiv) in THF (14.0 mL) dropwise over 3 minutes. The reaction was allowed to warm to 23° C. and stirred for 1 h before being cooled back down to 0° C. Then, TIPSCI (0.880 mL, 0.413 mmol, 1.5 equiv) was added to the reaction mixture dropwise over 5 minutes at 0° C. The reaction was allowed to warm 23° C. and stirred for 18 h, before being quenched with saturated ammonium chloride (2.0 mL) and deionized water (10.0 mL). The layers were transferred to a separatory funnel and the aqueous layer was extracted with diethyl ether (3×10 mL). The combined organic layers were then washed with brine (1×10 mL), dried over Na₂SO₄, filtered, and concentrated under reduced pressure. The crude residue was purified via flash chromatography (7:3 hexanes:EtOAc) to afford cycloadducts 40a and 40b (155 mg, 80% yield, 1:1 ratio of regioisomers) as a clear oil. Cycloadducts 40a and 40b: R_{f} 0.69 (7:3 hexanes:EtOAc); ¹H-NMR (500 MHz, CD₃CN, 60° C.): δ 7.69 (dd, J=5.1, 1.2, 1H), 7.65 (dd, J=9.3, 1.0, 1H), 7.63-7.59 (m, 2H), 7.37-7.25 (m, 13H), 7.23-7.19 (m, 4H), 7.16-7.08 (m, 6H), 7.06-7.04 (m, 3H), 5.45 (dd, J=3.2, 0.9, 1H), 5.30 (dd, J=3.3, 0.9, 1H), 5.08-5.04 (m, 4 H), 4.47-4.42 (m, 4H), 3.93 (s, 3H), 3.91 (s, 3H), 3.64-3.58 (m, 4H), 2.92-2.85 (m, 2H), 2.83 (t, J=6.1, 2H), 1.75-1.65 (m, 6H), 1.10 (d, J=2.9, 18H), 1.08 (d, J=2.9, 18H); ¹³C-NMR (125 MHz, CD₃CN, 60° C.): (56 of 64 observed) δ 160.9, 160.6, 156.3, 143.9, 141.7, 139.6, 139.3, 139.2, 138.6, 137.4, 135.5, 134.4, 132.5, 132.3, 132.23, 132.20, 131.9, 131.8, 131.3, 130.33, 130.27, 130.1, 129.6, 129.4, 129.14, 129.14, 129.02, 128.98, 128.92, 128.88, 128.8, 128.7, 128.6, 127.9, 127.6, 127.5, 126.8, 126.4, 121.6, 121.2, 117.0, 116.6, 116.1, 115.5, 108.5, 108.0, 67.71, 67.69, 56.41, 56.35, 46.2, 46.0, 43.1, 43.0, 18.7, 13.8; IR (film): 3066, 2947, 2867, 1701, 1243 cm⁻¹; HRMS-APCI (m/z) [M+H]⁺ calcd for C₄₃H₄₉N₂O₃SSi⁺, 701.3228; found, 701.3213.

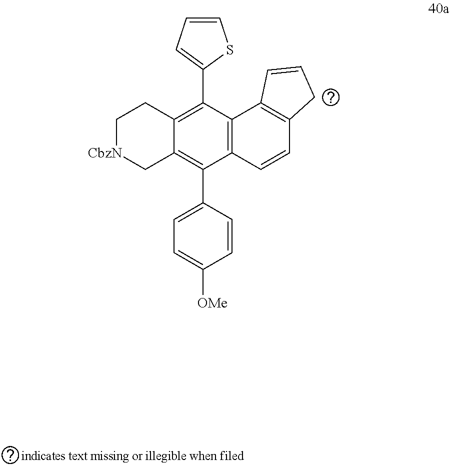 [SMILES: COc1ccc(-c2c3c(c(-c4cccs4)c4c5c(ccc24)CC=C5)CCN(C(=O)OCc2ccccc2)C3)cc1]

Cycloadduct 40a. Crystals suitable for X-ray diffraction studies were obtained by slow concentration of cycloadduct 40a from EtOAc (CCDC #1876925). Cycloadduct 40a: R_{f} 0.69 (7:3 hexanes:EtOAc); ¹H-NMR (500 MHz, CD₃CN, 60° C.): δ 7.70 (dd, J=5.2, 1.1, 1H), 7.61 (dd, J=9.4, 0.8, 1H), 7.37-7.25 (m, 6H), 7.25-7.20 (m, 2H), 7.16-7.09 (m, 4H), 7.06 (dd, J=3.4, 1.2, 1H), 5.45 (3.2, 0.8, 1H), 5.07 (s, 2H), 4.46 (s, 2H), 3.94 (s, 3H), 3.65-3.59 (m, 2H), 2.93-2.86 (m, 2H), 1.71 (sep, J=7.5, 3H), 1.10 (d, J=7.5, 18H); ¹³C-NMR (125 MHz, CD₃CN, 60° C.): (27 of 32 observed) δ 160.6, 156.4, 143.8, 139.6, 135.5, 132.5, 132.2, 130.3, 129.6, 129.1, 129.01, 128.96, 128.9, 128.8, 128.7, 127.9, 126.3, 121.6, 116.6, 115.5, 108.0, 67.7, 56.3, 46.0, 43.1, 18.6, 13.8; IR (film): 3030, 2947, 2868, 1702, 1245 cm⁻¹; HRMS-APCI (m/z) [M+H]⁺ calcd for C₄₃H₄₉N₂O₃SSi⁺, 701.3228; found, 701.3217.

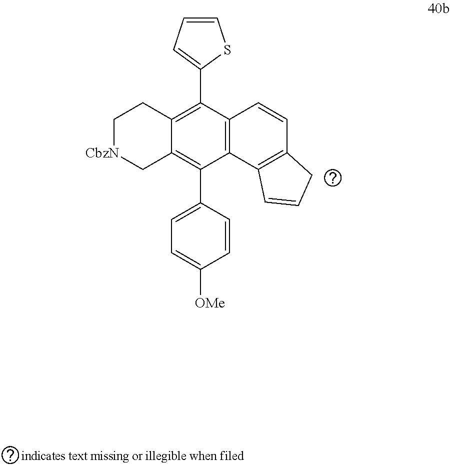 [SMILES: COc1ccc(-c2c3c(c(-c4cccs4)c4ccc5c(c24)C=CC5)CCN(C(=O)OCc2ccccc2)C3)cc1]

Cycloadduct 40b. Cycloadduct 40b: R_{f} 0.69 (7:3 hexanes:EtOAc); 1H-NMR (500 MHz, CD₃CN, 60° C.): δ 7.68-7.62 (m, 2H), 7.38-7.20 (m, 9H), 7.19-7.13 (m, 2H), 7.08-7.05 (m, 2H), 5.30 (dd, J=3.2, 0.8, 1H), 5.07 (s, 2H), 4.46 (s, 2H), 3.94 (s, 3H), 3.63 (t, J=6.2, 2H), 2.84 (t, J=6.2, 2H); ¹³C-NMR (125 MHz, CD₃CN, 60° C.): (31 of 32 observed) δ 160.9, 156.3, 141.7, 139.3, 138.6, 137.4, 134.4, 132.2, 131.9, 131.8, 131.3, 130.3, 130.1, 129.6, 129.4, 129.0, 128.7, 128.6, 127.6, 127.5, 126.8, 121.2, 117.0, 116.1, 108.5, 67.7, 56.4, 46.2, 43.0, 18.6, 13.8; IR (film): 3030, 2947, 2868, 1702, 1245 cm⁻¹; HRMS-APCI (m/z) [M+H]⁺ calcd for C₄₃H₄₉N₂O₃SSi⁺, 701.3228; found, 701.3216.

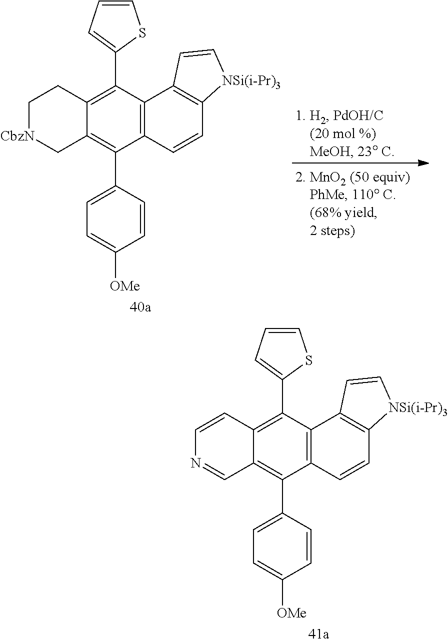

Indoloisoquinoline 41a. A solution of cycloadduct 40a (20 mg, 0.029 mmol, 1.0 equiv) and palladium hydroxide on carbon (20% wt %, 20 mg, 100% wt/wt relative to cycloadduct 40a) in methanol (4.0 mL) was purged with H₂ for 20 min. After stirring at 23° C. under an atmosphere of H₂ (1 atm) for 18 h, the reaction was diluted with CH₂Cl₂ (1.0 mL) and filtered through celite (monster pipette, ˜4 cm tall) with CH₂Cl₂ (10.0 mL) as the eluent, and concentrated under reduced pressure. The crude residue was added to a scintillation vial along with MnO₂ (125 mg, 1.43 mmol, 50 equiv) and toluene (0.5 mL). The reaction vial was heated to 110° C. and left to stir. After 18 h, the reaction mixture was cooled to 23° C., filtered through celite (monster pipette, ˜4 cm tall) with CH₂Cl₂ (10.0 mL) as the eluent, and concentrated under reduced pressure. Purification of the crude residue via preparative thin layer chromatography (3:1 hexanes:EtOAc) afforded indoloisoquinoline 41a (11.0 mg, 68% yield, over 2 steps) as a yellow amorphous solid. Indoloisoquinoline 41a: R_{f} 0.54 (9:1 PhH:EtOAc); ¹H-NMR (500 MHz, CDCl₃): δ 9.13 (d, J=0.8, 1H), 8.37 (d, J=6.2, 1H), 7.70 (dd, J=5.2, 1.2, 1H), 7.63 (dd, J=9.6, 0.8, 1H), 7.57 (dd, J=6.2, 1.0, 1H), 7.53 (d, J=9.6, 1H), 7.45-7.41 (m, 2H), 7.39 (dd, J=5.2, 3.4, 1H), 7.21 (dd, J=3.3, 1.2, 1H), 7.17-7.14 (m, 2H), 7.10 (d, J=3.2), 5.66 (dd, J=3.2, 0.9, 1H), 3.97 (s, 3H), 1.70-1.63 (m, 3H), 1.12 (d, J=7.6, 18H); ¹³C-NMR (125 MHz, CDCl₃): δ 159.5, 153.2, 141.3, 140.9, 140.6, 138.4, 133.5, 132.5, 132.4, 130.7, 130.1, 128.9, 128.7, 128.5, 128.0, 127.2, 124.5, 124.4, 123.9, 121.8, 118.0, 117.3, 114.0, 108.8, 55.6, 18.3, 13.1; IR (film): 3068, 2949, 2928, 1392, 1247 cm⁻¹; HRMS-APCI (m/z) [M+H]⁺ calcd for C₃₅H₃₉N₂OSSi⁺, 563.2547; found, 563.2545.

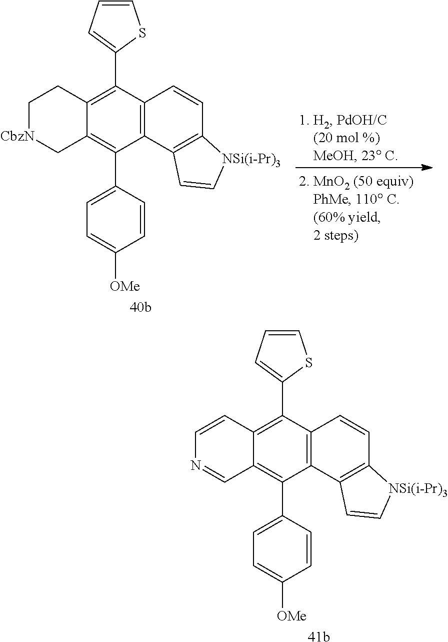

Indoloisoquinoline 41b. A solution of cycloadduct 40b (20 mg, 0.029 mmol, 1.0 equiv) and palladium hydroxide on carbon (20% wt %, 20 mg, 100% wt/wt relative to cycloadduct 40b) in methanol (4.0 mL) was purged with H₂ for 20 min. After stirring at 23° C. under an atmosphere of H₂ (1 atm) for 18 h, the reaction was diluted with CH₂Cl₂ (1.0 mL) and filtered through celite (monster pipette, ˜4 cm tall) with CH₂Cl₂ (10.0 mL) as the eluent, and concentrated under reduced pressure. The crude residue was added to a scintillation vial along with MnO₂ (125 mg, 1.43 mmol, 50 equiv) and toluene (0.5 mL). The reaction vial was heated to 110° C. and left to stir. After 18 h, the reaction mixture was cooled to 23° C., filtered through celite (monster pipette, ˜4 cm tall) with CH₂Cl₂ (10.0 mL) as the eluent, and concentrated under reduced pressure. Purification of the crude residue via preparative thin layer chromatography (3:1 hexanes:EtOAc) afforded indoloisoquinoline 41b (9.7 mg, 60% yield, over 2 steps) as a yellow amorphous solid. Indoloisoquinoline 41b: R_{f} 0.54 (9:1 PhH:EtOAc); ¹H-NMR (500 MHz, CDCl₃): δ 9.12 (d, J=0.9, 1H), 8.35 (d, J=6.1, 1H), 7.72 (dd, J=9.6, 0.8, 1H), 7.66-7.62 (m, 2H), 7.57 (dd, J=6.2, 1.0, 1H), 7.43-7.40 (m, 2H), 7.33 (dd, J=5.2, 3.4, 1H), 7.24 (dd, J=3.3, 1.2, 1H), 7.22-7.19 (m, 2H), 7.04 (d, J=3.2), 5.52 (dd, J=3.1, 0.8, 1H), 4.01 (s, 3H), 1.66 (sept, J=7.7, 3H), 1.12 (d, J=7.7, 18H); ¹³C-NMR (125 MHz, CDCl₃): δ 159.8, 153.0, 140.3, 139.2, 137.7, 137.2, 132.2, 131.94, 131.87, 131.0, 129.7, 128.7, 127.7, 127.4, 127.1, 126.9, 125.9, 125.5, 120.8, 119.0, 118.2, 114.8, 109.1, 55.6, 18.3, 13.1; IR (film): 3065, 2949, 2868, 1383, 1245 cm⁻¹; HRMS-APCI (m/z) [M+H]⁺ calcd for C₃₅H₃₉N₂OSSi⁺, 563.2547; found, 563.2537.

F. Synthesis of Donor-Acceptor Fluorophore and Polymer.

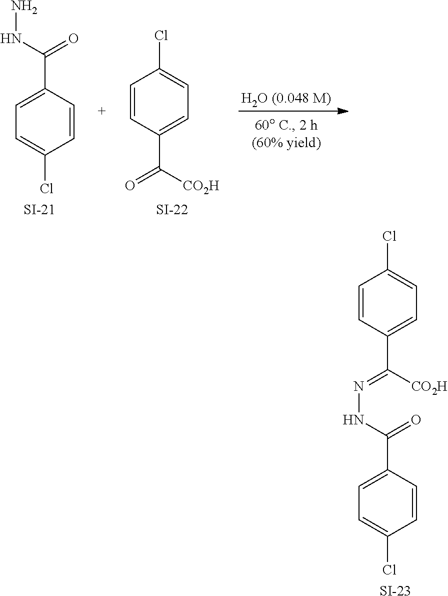

Hydrazone Sl-23. Followed General Procedure A using hydrazide Sl-21 (1.73 g, 9.37 mmol, 1.0 equiv) afforded hydrazone Sl-23 (2.00 g, 63% yield) as a white solid. Hydrazone Sl-23: mp 178-180° C.; R_{f} 0.56 (9:1 EtOAc:MeOH); ¹H-NMR (500 MHz, DMSO-d₆, 60° C.): δ 12.72 (br s, 1H), 7.87 (d, J=8.3, 2H), 7.72 (d, J=8.3, 2H), 7.62 (d, J=8.3, 2H), 7.49 (d, J=8.3, 2H); ¹³C-NMR (125 MHz, DMSO-d₆, 60° C.): δ 163.2, 141.6, 136.9, 134.0, 133.4, 131.4, 130.5, 129.7, 129.4, 128.6, 128.0; IR (film): 3359, 3087, 1729, 1662, 1093 cm⁻¹; HRMS-APCI (m/z) [M+H]⁺ calcd for C₁₅H₁₁Cl₂N₂O₃⁺, 337.0141; found, 337.0130.

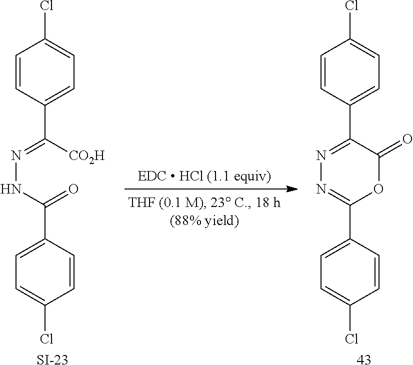

Oxadiazinone 43. Followed General Procedure A using hydrazone SI-23 (1.80 g, 5.34 mmol, 1.0 equiv) afforded oxadiazinone 43 (1.50 g, 88% yield) as a yellow solid after recrystallization from hot acetone. Oxadiazinone 43: mp 229-233° C.; R_{f} 0.64 (9:1 EtOAc:MeOH); ¹H-NMR (500 MHz, CDCl₃): δ 8.34-8.30 (m, 2H), 8.24-8.21 (m, 2H), 7.56-7.52 (m, 2H), 7.51-7.48 (m, 2H); ¹³C-NMR (125 MHz, CDCl₃): δ 157.2, 152.0, 147.9, 140.7, 138.9, 130.5, 129.8, 129.7, 129.4, 129.2, 126.0; IR (film): 3099, 1757, 1595, 1151, 1094 cm⁻¹; HRMS-APCI (m/z) [M+H]⁺ calcd for C₁₅H₉Cl₂N₂O₂⁺, 319.0036; found, 319.0032.

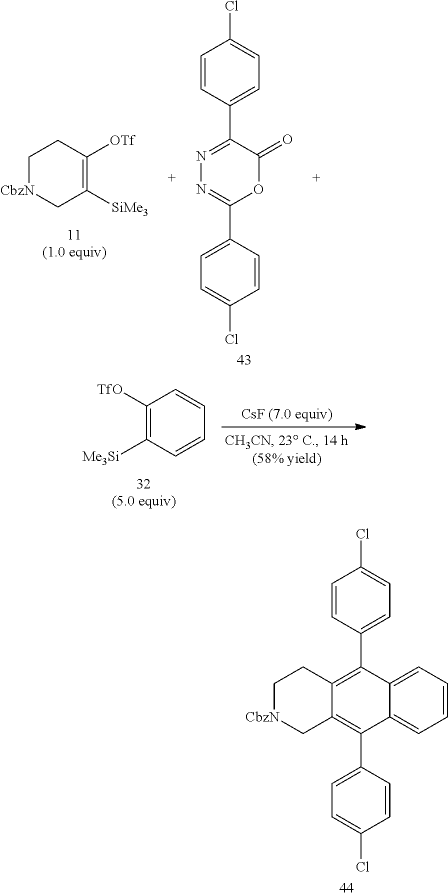

Tricycle 44. A solution of silyl triflate 11 (25.0 mg, 0.057 mmol, 1.0 equiv), oxadiazinone 43 (36.5 mg, 0.114 mmol, 2.0 equiv), and silyl triflate 32 (85.3 mg, 0.286 mmol, 5.0 equiv) in acetonitrile (5.7 mL) was purged with nitrogen for 10 min. Then, CsF (60.8 mg, 0.400 mmol, 7.0 equiv) was added and the reaction was allowed to stir at 23° C. for 14 h. Upon completion of the reaction, it was filtered through celite (monster pipette, ˜4 cm tall) using CH₂Cl₂ (˜10 mL) as the eluent and concentrated under reduced pressure. The crude residue was purified using preparative thin layer chromatography (100% benzene) to afford tricycle 44 (17.9 mg, 58% yield) as a yellow oil. Tricycle 44: R_{f} 0.58 (17:3 hexanes:EtOAc); ¹H-NMR (500 MHz, CD₃CN, 60° C.): δ 7.60-7.55 (m, 4H), 7.42-7.23 (m, 13H), 5.06 (s, 2H), 4.46 (s, 2H), 3.59 (t, J=6.0, 2H), 2.75 (t, J=6.3, 2H); ¹³C-NMR (125 MHz, CD₃CN, 60° C.): δ 156.3, 138.8, 138.5, 138.1, 137.9, 136.4, 134.6, 134.3, 133.1, 132.99, 132.96, 132.8, 132.4, 131.8, 130.1, 129.97, 129.97, 129.96, 129.95, 129.94, 129.63, 129.62, 129.61, 129.60, 129.59, 129.59, 129.0, 128.7, 127.1, 127.03, 127.00, 126.9, 67.8, 45.7, 43.0, 28.7; IR (film): 3065, 3033, 2935, 2891, 1699 cm⁻¹; HRMS-APCI (m/z) [M]^{⋅+} calcd for C₃₃H₂₅Cl₂NO₂⁺, 537.1257; found, 537.1204.

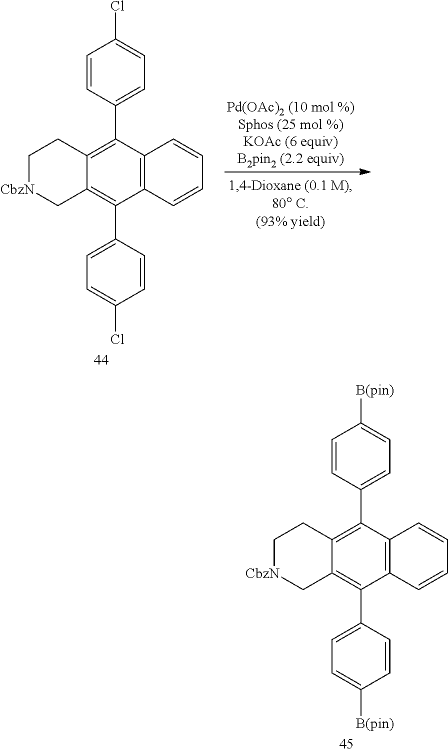

Boronic Ester 45. To a vial was added tricycle 44 (20 mg, 0.037 mmol, 1.0 equiv), B₂pin₂ (19.8 mg, 0.078 mmol, 2.2 equiv), KOAc (21.9 mg, 0.233 mmol, 6.0 equiv), Pd(OAc)₂ (0.8 mg, 0.0037 mmol, 10 mol %), and SPhos (3.8 mg, 0.0093 mmol, 25 mol %), and the vial was purged with nitrogen for 30 min. Then, 1,4-dioxane (3.7 mL) was added and the reaction was heated to 80° C. After stirring for 18 h, the reaction was cooled to 23° C. and concentrated under reduced pressure. The crude residue was purified via flash chromatography (Yamazen SiO₂, 3:1 hexanes:EtOAc) to afford boronic ester 45 (25.0 mg, 93% yield) as a white foam. Boronic Ester 45: R_{f} 0.29 (17:3 hexanes:EtOAc); ¹H-NMR (500 MHz, CD₃CN, 60° C.): δ 7.95-7.88 (m, 4H), 7.40-7.20 (m, 13H), 5.04 (s, 2H), 4.46 (s, 2H), 3.57 (t, J=6.3, 2H), 2.76 (t, J=6.3, 2H), 1.42 (s, 12H), 1.40 (s, 12H); ¹³C-NMR (125 MHz, CD₃CN, 60° C.): δ (29 of 35 signals observed) 156.3, 143.2, 142.5, 138.8, 138.5, 137.3, 136.0, 135.9, 132.7, 132.6, 132.3, 131.4, 131.0, 130.8, 129.6, 128.9, 128.6, 127.13, 127.09, 126.9, 126.7, 85.3, 85.2, 67.7, 45.8, 43.0, 28.7, 25.52, 25.48; IR (film): 3032, 2978, 2938, 1705, 1358 cm⁻¹; HRMS-APCI (m/z) [M+K]⁺ calcd for C₄₅H₄₉B₂NO₆K⁺, 760.3378; found, 760.3376.

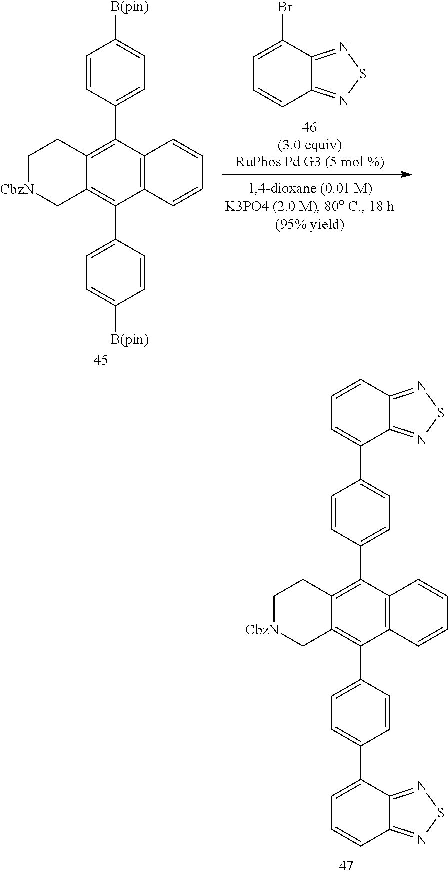

Donor-Acceptor 47. A vial was charged with boronic ester 45 (20 mg, 0.028 mmol, 1.0 equiv), 4-bromobenzothiadiazole (46) (17.9 mg, 0.083 mmol, 3.0 equiv), and RuPhos Pd G3 (1.2 mg, 0.0014 mmol, 5 mol %), and then evacuated and backfilled with nitrogen three times. A separate flask containing a 2.0 M solution of aqueous K₃PO₄ was sparged with nitrogen for 1 h. To the vial, was then added 1,4-dioxane (2.8 mL) and the reaction was heated to 80° C. After 10 min, K₃PO₄ (2.0 M solution, 0.28 mL) was added and the reaction was allowed to stir at 80° C. After 18 h, the reaction was cooled to 23° C., filtered over celite (monster pipette, ˜4 cm tall) using CH₂Cl₂ (˜10 mL) as the eluent, and concentrated under reduced pressure. The crude residue was purified via flash chromatography (Yamazen SiO₂, 17:3 hexanes:EtOAc) to afford donor-acceptor 47 (19.5 mg, 95% yield) as an off-white solid. Donor-acceptor 47: mp 237-238° C.; R_{f} 0.42 (3:1 hexanes:EtOAc); ¹H-NMR (500 MHz, DMSO-d₆, 70° C.): δ 8.24 (d, J=8.2, 4H), 8.18-8.12 (m, 2H), 8.06-8.01 (m, 2H), 7.93-7.87 (m, 2H), 7.58-7.41 (m, 8H), 7.30-7.10 (m, 5H), 5.05 (s, 2H), 4.61 (s, 2H), 3.64 (t, J=6.1, 2H), 2.88 (t, J=6.1, 2H); ¹³C-NMR (125 MHz, DMSO-d₆, 70° C.): δ (37 of 39 observed) 155.0, 154.9, 154.3, 152.59, 152.56, 138.1, 137.4, 136.6, 136.5, 135.9, 135.7, 135.2, 132.8, 132.7, 131.0, 130.9, 130.5, 130.0, 129.87, 129.86, 129.8, 129.12, 129.08, 127.9, 127.8, 127.7, 127.0, 125.51, 125.49, 125.3, 120.32, 120.27, 65.9, 44.2, 41.4, 28.6; IR (film): 3071, 3032, 2925, 2854, 1700 cm⁻¹; HRMS-APCI (m/z) [M+K]⁺ calcd for C₄₅H₃₂N₅O₂S₂⁺, 738.1992; found, 738.1983.

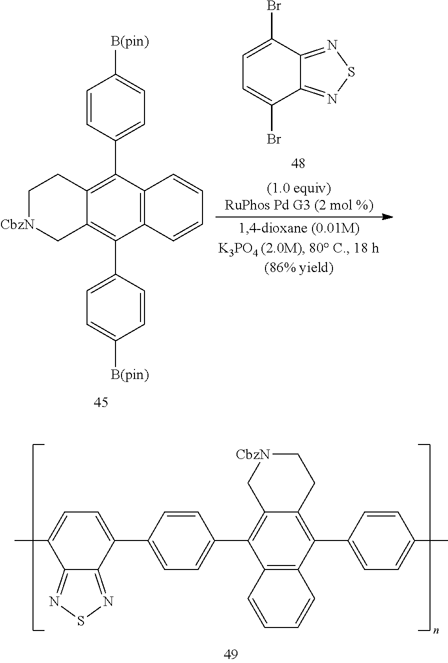

Polymer 49. A vial was charged with boronic ester 45 (2.01 mg, 0.0291 mmol, 1.00 equiv), 4,7-dibromobenzothiadiazole (48) (8.56 mg, 0.0291 mmol, 1.00 equiv), and RuPhos Pd G3 (0.487 mg, 0.000582 mmol, 2.00 mol %), and then evacuated and backfilled with nitrogen three times. A separate flask containing a 2.0 M solution of aqueous K₃PO₄ was sparged with nitrogen for 1 h. To the vial, was added 1,4-dioxane (2.91 mL) and the reaction was heated to 80° C. After 10 min, K₃PO₄ (2.0 M solution, 0.100 mL) was added and the reaction was allowed to stir at 80° C. After 18 h, the reaction was cooled to 23° C., filtered over celite (monster pipette, ˜4 cm tall) using CH₂Cl₂ (˜10 mL) as the eluent, and concentrated under reduced pressure. The crude residue was dissolved in CH₂Cl₂ (0.300 mL), then crashed out with methanol (5.00 mL) and filtered to afford polymer 49 (15.8 mg, 86% yield) as an off-white solid. Polymer 49: ¹H-NMR (500 MHz, CDCl₃): δ 8.37-7.84 (m), 7.81-7.08 (m), 5.26-5.01 (m), 4.79-4.53 (m), 3.79-3.58 (m), 3.05-2.76 (m).

Polymer Characterization. Near-monodisperse poly(styrene) standards (Polymer Laboratories) were employed for calibration and Mw, Mn, and DM were calculated from refractive index. Polymer 49 was found to have a Mw of 2.40 kDa, a Mn of 1.72 kDa, and a DM of 1.39.

Materials and Methods Related to Triphenylene Scaffold PAH scaffolds.

Unless stated otherwise, reactions were conducted in flame-dried glassware under an atmosphere of nitrogen or argon and commercially obtained reagents were used as received. Anhydrous solvents were either freshly distilled or passed through activated alumina columns, unless otherwise stated. Reaction temperatures were controlled using an IKAmag temperature modulator, and unless stated otherwise, reactions were performed at room temperature (approximately 23° C.). Cesium Fluoride (CsF), and Bis(dibenzylidenacetone)palladium(0) (Pd(dba)₂), and Palladium(II) acetate (Pd(OAc)₂) were obtained from Strem Chemicals and stored in a desiccator. Potassium hexafluorophosphate (KPF₆) was obtained from Oakwood Chemicals and used as received. Tri(o-tolyl)phosphine (P(o-tolyl)₃) and decolorizing carbon were obtained from Sigma-Aldrich. 2-Bromobiphenyl (17) was obtained from Combi-Blocks and purified by flash chromatography prior to use. Bromobiaryl SI-7 was obtained from Combi-Blocks. Bromobiaryls SI-3 (Zhang, Q.-W.; et al., Rhodium-Catalyzed Intramolecular C—H Silylation by Silacyclobutanes. Angew. Chem., Int. Ed. 2016, 55, 6319-6323, the disclosure of which is incorporated herein by reference), SI-5 (Wang, T.-F.; et al. Easily Accessible 2-(2-Bromophenyl)-4,4,5,5-tetramethyl-[1,3,2]dioxaborolane for Suzuki-Miyaura Reactions. J. Chin. Chem. Soc. 2007, 54, 811-816, the disclosure of which is incorporated herein by reference), SI-9 (Wu, D.; et al. Synthesis of 1,3-Azaphospholes with Pyrrolo[1,2-a]quinolone Skeleton and Their Optical Applications. Org. Lett. 2018, 20, 4103-4106, the disclosure of which is incorporated herein by reference), SI-11 (Wong, S. M.; et al. Preparation of 2-(2-(Dicyclohexylphosphino)phenyl)-1-methyl-1H-indole (CM-phos) Org. Synth. 2016, 93, 14-28, the disclosure of which is incorporated herein by reference), and SI-13 (Panteleev, J.; et al. C—H Bond Functionalization in the Synthesis of Fused 1,2,3-Triazoles. Org. Lett. 2010, 12, 5092-5095, the disclosure of which is incorporated herein by reference) were prepared according to literature procedures. The silyl triflate precursors to N-Me-4,5-indolyne (Bronner, S. M.; et al. Indolynes as Electrophilic Indole Surrogates: Fundamental Reactivity and Synthetic Applications. Org. Lett. 2009, 11, 1007-1010, the disclosure of which is incorporated herein by reference), N-Boc-4,5-indolyne (Im, G.-Y. J.; et al. Indolyne Experimental and Computational Studies: Synthetic Applications and Origins of Selectivities of Nucleophilic Additions. J. Am. Chem. Soc. 2010, 132, 17933-17944, the disclosure of which is incorporated herein by reference), N-Me-5,6-indolyne, and N-Cbz-3,4,-piperidyne (McMahon, T. C.; et al. Generation and Regioselective Trapping of a 3,4-Piperidyne for the Synthesis of Functionalized Heterocycles. J. Am. Chem. Soc. 2015, 137, 4082-4085 the disclosure of which is incorporated herein by reference) were prepared following literature procedures. The synthesis of the silyl triflate precursor to N-Me-2,3-carbazolyne will not be discussed in this report. Regioisomeric ratios for annulation products were determined from ¹H-NMR spectra of the crude mixtures. Thin-layer chromatography (TLC) was conducted with EMD gel 60 F254 pre-coated plates (0.25 mm for analytical chromatography and 0.50 mm for preparative chromatography) and visualized using UV. Silicycle Siliaflash P60 (particle size 0.040-0.063 mm) was used for flash column chromatography. ¹H NMR spectra were recorded on Bruker spectrometers (at 400, 500 and 600 MHz) and are reported relative to residual solvent signals. Data for ¹H NMR spectra are reported as follows: chemical shift (δ ppm), multiplicity, coupling constant (Hz), integration. Data for ¹³C NMR are reported in terms of chemical shift (at 101 and 125 MHz). IR spectra were recorded on a Perkin-Elmer UATR Two FT-IR spectrometer and are reported in terms of frequency absorption (cm⁻¹). DART-MS spectra were collected on a Thermo Exactive Plus MSD (Thermo Scientific) equipped with an ID-CUBE ion source and a Vapur Interface (lonSense Inc.). Both the source and MSD were controlled by Excalibur software v. 3.0. The analyte was spotted onto OpenSpot sampling cards (lonSense Inc.) using CH₂Cl₂ as the solvent. Ionization was accomplished using UHP He plasma with no additional ionization agents. The mass calibration was carried out using Pierce LTQ Velos ESI (+) and (−) Ion calibration solutions (Thermo Fisher Scientific).

### Experimental Procedures

A. Scope of Methodology

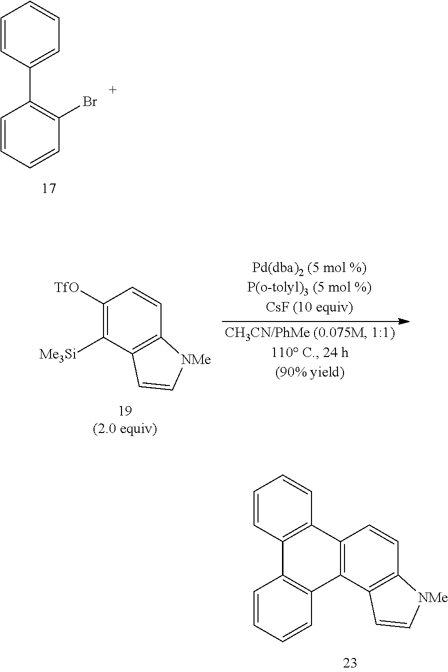 [SMILES: Brc1ccccc1-c1ccccc1 ; Cn1ccc2c([Si](C)(C)C)c(OS(=O)(=O)C(F)(F)F)ccc21 ; Cn1ccc2c3c4ccccc4c4ccccc4c3ccc21]

Representative Procedure (Palladium-catalyzed annulation of indolyne precursor 19 with 2-bromobiphenyl (17) is used as an example). Indole 23. A 1-dram vial was charged with Pd(dba)₂ (3.7 mg, 0.064 mmol, 5 mol %). Next, toluene (0.86 mL), P(o-tolyl)₃ (2.0 mg, 0.064 mmol, 5 mol %), 2-bromobiphenyl (17) (30.0 mg, 0.129 mmol, 1.0 equiv), silyl triflate 19 (90.5 mg, 0.257 mmol, 2.0 equiv), and acetonitrile (0.86 mL) were added followed by an oven-dried magnetic stirbar and then CsF (195 mg, 1.29 mmol, 10.0 equiv). The vial was sealed with a Teflon-lined screw cap and stirred at 110° C. for 24 h. After allowing to cool to 23° C., the mixture was transferred with CH₂Cl₂ (10 mL) and H₂O (2 mL) to a 150 mL separatory funnel containing brine (15 mL). The layers were separated and the aqueous layer was extracted with CH₂Cl₂ (3×15 mL). The combined organic layers were dried over MgSO₄, filtered, and concentrated in vacuo. The resulting crude product was purified by flash chromatography (100% Hexanes→200:1 Hexanes:EtOAc) to yield annulation product 23 (90% yield, average of two experiments) as an off-white solid. Indole 23: mp: 139-144° C.; R_{f} 0.41 (4:1 hexanes:EtOAc); ¹H NMR (500 MHz, CDCl₃): δ 9.24 (d, J=8.0, 1H), 8.78 (d, J=8.0, 1H), 8.72 (t, J=7.0, 2H), 8.57 (d, J=9.0, 1H), 7.75 (t, J=7.4, 1H), 7.73-7.62 (m, 4H), 7.53 (d, J=2.8, 1H), 7.28 (d, J=3.0, 1H), 3.92 (s, 3H); ¹³C NMR (125 MHz, CDCl₃): δ 136.4, 131.3, 131.2, 130.4, 128.9, 128.8, 127.17, 127.15, 126.7, 126.4, 125.9, 124.6, 124.1, 123.8, 123.6, 123.3, 123.3, 117.7, 110.5, 104.0, 33.3; IR (film): 3069, 2924, 2850, 1514, 1492, 1441, 1417, 1351, 1248, 754, 740, 718 cm⁻¹; HRMS-APCI (m/z) [M+H]⁺ calcd for C₂₁H₁₆N⁺, 282.12773; found 282.12717.

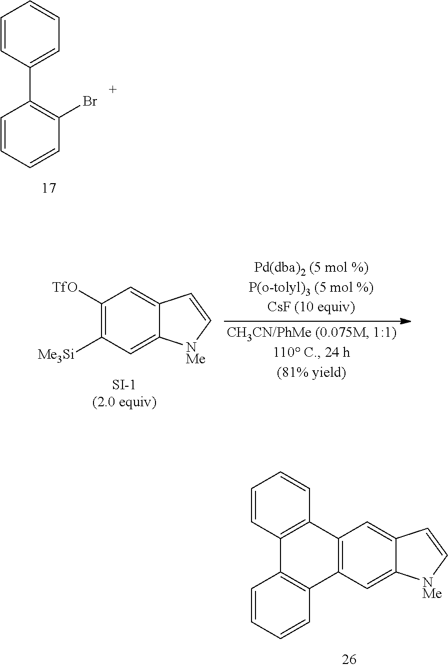 [SMILES: Brc1ccccc1-c1ccccc1 ; Cn1ccc2cc(OS(=O)(=O)C(F)(F)F)c([Si](C)(C)C)cc21 ; Cn1ccc2cc3c4ccccc4c4ccccc4c3cc21]

Indole 26. Purification by flash chromatography (hexanes to 1:1 hexanes/benzene) afforded indole 26 (81% yield, average of two experiments) as an off-white solid. Indole 26: R_{f} 0.25 (9:1 hexanes:EtOAc); ¹H NMR (500 MHz, CDCl₃): δ 8.92 (s, 1H), 8.73 (d, J=8.3, 2H), 8.62 (t, J=7.7, 2H), 8.51 (s, 1H), 7.66-7.56 (m, 4H), 7.27 (d, J=2.6, 1H), 6.70 (dd, J=0.8, 3.1, 1H), 4.00 (s, 3H); ¹³C NMR (125 MHz, CDCl₃): (27 of 28 signals observed) δ 137.4, 132.0, 131.4, 131.2, 129.6, 129.3, 129.0, 127.3, 127.1, 126.6, 126.1, 125.7, 123.56, 123.55, 123.4, 123.22, 123.15, 115.1, 102.3, 101.1, 33.2; IR (film): 3081, 2928, 2811, 1628, 1601, 1520, 1446, 1218, 1085, 754 cm⁻¹; HRMS-APCI (m/z) [M]⁺ calcd for C₂₁H₁₆N⁺, 281.11990; found 281.12065.

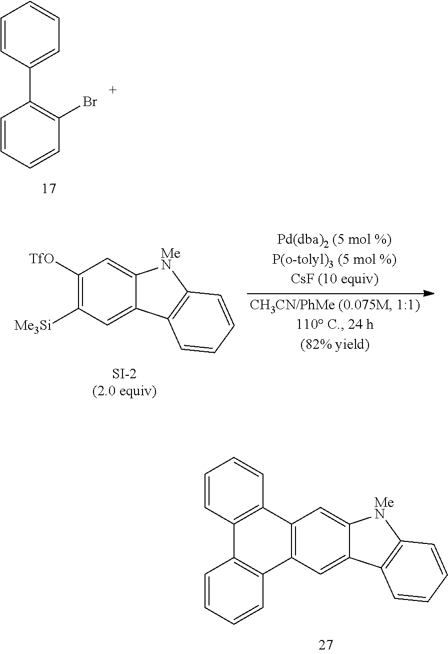 [SMILES: Brc1ccccc1-c1ccccc1 ; Cn1c2ccccc2c2cc([Si](C)(C)C)c(OS(=O)(=O)C(F)(F)F)cc21 ; Cn1c2ccccc2c2cc3c4ccccc4c4ccccc4c3cc21]

Carbazole 27. Purification by flash chromatography (100% Hexanes to 1:1 Hexanes:Benzene) afforded carbazole 27 (82% yield) as an off-white solid. Carbazole 27: R_{f} 0.74 (4:1 Hexanes:EtOAc); ¹H NMR (400 MHz, CDCl₃): δ 9.38 (s, 1H), 8.86 (d, J=8.2, 1H), 8.81 (d, J=7.8, 1H), 8.68 (td, J=8.6, 1.5, 2H), 8.54 (s, 1H), 8.31 (dt, J=7.8, 0.9, 1H), 7.72-7.60 (m, 4H), 7.63 (td, J=7.8, 1.1, 1H), 7.58 (td, J=7.8, 1.1, 1H), 7.47 (d, J=8.1, 1H), 7.33 (td, J=7.4, 0.8, 1H), 4.03 (s, 3H); ¹³C NMR (100 MHz, CDCl₃): (27 of 28 signals observed) δ 142.9, 141.3, 131.1, 130.6, 130.0, 128.85, 128.78, 127.4, 127.1, 127.0, 126.8, 126.1, 123.9, 123.6, 123.5, 123.4, 123.1, 123.0, 122.9, 120.8, 119.2, 114.8, 108.5, 101.1, 29.3; IR (film): 3049, 2923, 2854, 1638, 1603, 1500, 1443, 1258, 754 cm⁻¹; HRMS-APCI (m/z) [M]⁺ calcd for C₂₅H₁₇N⁺, 331.13555; found 331.13609.

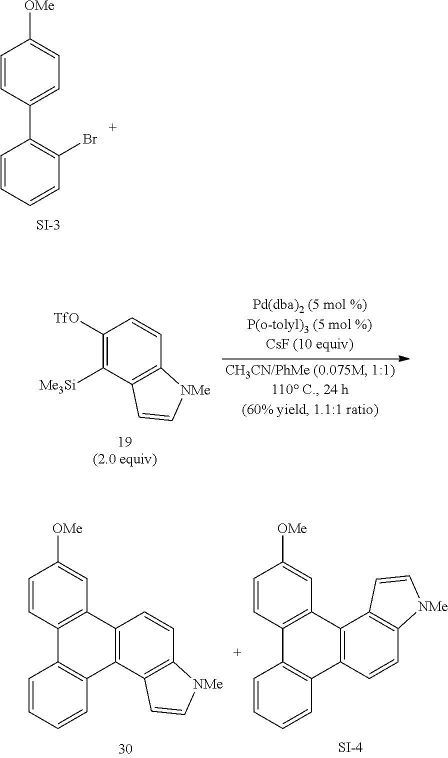

Indoles 30 and SI-4. Purification by flash chromatography (50:1 Hexanes:EtOAc) afforded an inseparable mixture of indoles 30 and SI-4 (60% yield, average of two experiments, 1.1:1 ratio) as a white solid. Indoles 30 and SI-4: R_{f} 0.27 (4:1 Hexanes:EtOAc); ¹H-NMR (500 MHz, C₆D₆, combined): δ 9.42 (dd, J=8.3, 1.3, 1H), 8.93 (d, J=2.6, 1H), 8.69 (dd, J=7.9, 1.3, 1H), 8.57-8.50 (m, 5H), 8.48 (d, J=9.0, 1H), 8.22 (d, J=2.5, 1H), 7.60-7.47 (m, 6H), 7.30-7.26 (m, 3H), 7.21 (dd, J=9.2, 2.6, 1H), 6.72 (d, J=6.7, 1H), 6.69 (d, J=3.2, 1H), 3.61 (s, 3H), 3.55 (s, 3H), 3.001 (s, 3H), 2.995 (s, 3H); ¹³C-NMR (500 MHz, C₆D₆, combined): δ 159.6, 159.3, 136.7, 136.6, 133.32, 133.31, 131.2, 131.0, 130.8, 129.7, 128.6, 128.5, 127.6, 126.6, 126.4, 126.2, 126.0, 125.8, 125.4, 125.3, 125.2, 125.1, 125.0, 124.6, 124.5, 124.4, 124.0, 123.6, 123.27, 123.25, 118.1, 118.0, 115.5, 115.0, 110.8, 110.5, 110.1, 106.5, 104.5, 104.0, 55.0, 54.9, 32.31, 32.28; IR (film): 2934, 2834, 1614, 1510, 1414, 1246, 1227 cm⁻¹; HRMS-APCI (m/z) [M+H⁺] calcd for C₂₂H₁₈NO⁺, 312.13829; found 312.13897.

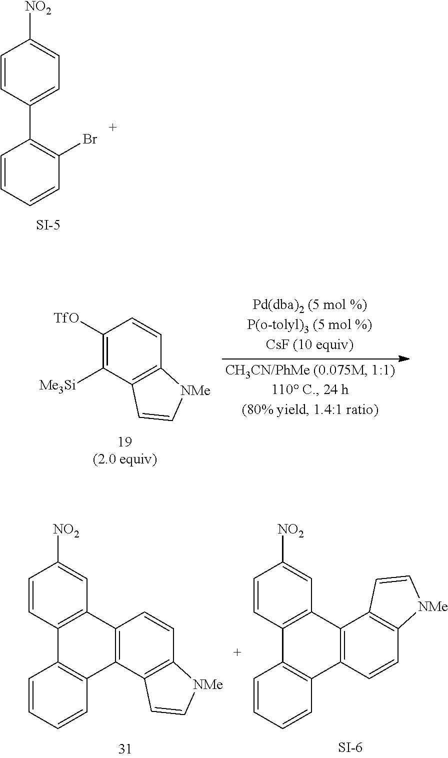

Indoles 31 and SI-6. Purification by flash chromatography (50:1 Hexanes:EtOAc→20:1 Hexanes:EtOAc) afforded an inseparable mixture of indoles 31 and SI-6 (80% yield, average of two experiments, 1.4:1 ratio) as a yellow solid. Indoles 31 and SI-6: R_{f} 0.45 (4:1 hexanes:EtOAc); ¹H-NMR (600 MHz, CDCl₃, major): δ 10.10 (d, J=2.4, 1H), 8.79 (d, J=9.1, 1H), 8.71 (d, J=8.3, 1H), 8.66 (d, J=8.2, 1H), 8.55 (d, J=9.2, 1H), 8.41 (dd, J=9.0, 2.3, 1H), 7.78-7.72 (m, 2H), 7.68-7.65 (ddd, J=8.1, 7.0, 1.2, 1H), 7.54 (d, J=3.1, 1H), 7.39 (d, J=3.2, 1H), 4.00 (s, 3H); ¹H-NMR (600 MHz, CDCl₃, minor): δ 9.54 (d, J=2.3, 1H), 9.21 (d, J=8.3, 1H), 8.75 (d, J=9.1, 1H), 8.70 (d, J=8.3, 1H), 8.55 (d, J=8.9, 1H), 8.35 (dd, J=9.0, 2.3, 1H), 7.82 (ddd, J=8.1, 7.0, 1.3, 1H), 7.76-7.71 (m, 2H), 7.51 (d, J=3.1, 1H), 7.36 (d, J=3.2, 1H), 4.00 (s, 3H); ¹³C-NMR (125 MHz, CDCl₃, combined): δ 146.4, 146.0, 136.8, 136.4, 134.8, 133.3, 132.51, 132.48, 131.3, 131.0, 129.9, 129.4, 129.0, 128.7, 128.6, 127.4, 127.3, 126.8, 126.4, 125.2, 124.6, 124.4, 124.21, 124.17, 124.14, 123.8, 123.60, 123.55, 123.5, 123.1, 122.8, 119.9, 119.53, 119.47, 117.5, 117.4, 111.7, 111.1, 104.0, 103.4, 33.4, 33.3; IR (film): 2919, 2852, 1597, 1515, 1346, 854, 747 cm⁻¹; HRMS-APCI (m/z) [M+H⁺] calcd for C₂₁H₁₅NO₂⁺, 327.11280; found 327.11387.

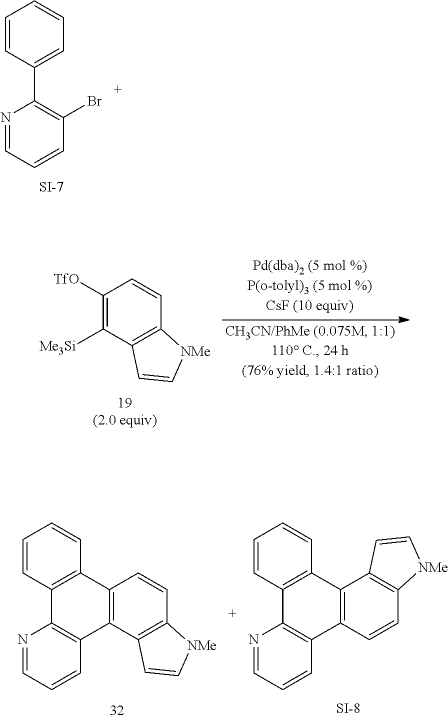

Indoles 32 and SI-8. Purification by flash chromatography (100% Hexanes→50:1 Hexanes:EtOAc→9:1 Hexanes:EtOAc) afforded an inseparable mixture of indoles 32 and SI-8 (76% yield, 1.4:1 ratio, average of two experiments) as a pale yellow solid. Indole 32: R_{f} 0.34 (4:1 hexanes:EtOAc); ¹H-NMR (500 MHz, CDCl₃): δ 9.40 (ddd, J=8.2, 4.7, 1.4, 2H), 8.99 (dd, J=4.3, 1.6, 1H), 8.66 (d, 8.2, 1H), 8.54 (d, 8.5, 1H), 7.76 (ddd, J=8.1, 6.9, 1.6, 1H), 7.71 (ddd, J=8.3, 6.8, 1.2, 1H), 7.65 (d, J=9.0, 1H), 7.64-7.60 (m, 1H), 7.38 (d, J=2.9, 1H), 7.27 (d, J=3.2, 1H), 3.91 (s, 3H). Indole SI-8: R_{f} 0.34 (4:1 hexanes:EtOAc); ¹H-NMR (500 MHz, CDCl₃): δ 9.46 (dd, J=8.1, 1.3, 1H), 9.18 (d, J=8.2, 1H), 8.95 (dd, J=4.3, 1.6, 1H), 8.89 (dd, J=8.4, 1.3, 1H), 8.41 (d, J=9.0, 1H), 7.83 (ddd, J=8.2, 7.0, 1.7, 1H), 7.77 (ddd, J=7.2, 5.6, 1.2, 1H), 7.64-7.60 (m, 1H), 7.55 (dd, J=8.3, 4.3, 1H), 7.53 (d, J=3.1, 1H), 7.29 (d, J=3.2, 1H), 3.90 (s, 3H); ¹³C-NMR (100 MHz, CDCl₃, combined): 147.69, 147.66, 146.9, 145.6, 136.6, 136.3, 134.2, 132.8, 132.7, 131.4, 131.0, 130.1, 129.3, 129.2, 128.8, 128.4, 126.6, 126.4, 126.2, 126.1, 125.8, 125.4, 125.2, 124.6, 124.2, 124.0, 123.9, 123.2, 122.9, 122.8, 122.1, 121.5, 117.7, 117.3, 111.1, 110.6, 104.0, 103.2, 33.3 (2C); IR (film): 3059, 2920, 1739, 1609, 1579, 1513, 1477, 1444, 1418, 1399, 1349, 1290, 1241 cm⁻¹; HRMS-APCI (m/z) [M+H⁺] calcd for C₂₀H₁₅N₂⁺, 283.12297; found 283.11932.

The structure of 32 was verified by 2D-NOESY and 2D-COSY of the mixture, as the following interactions were observed:

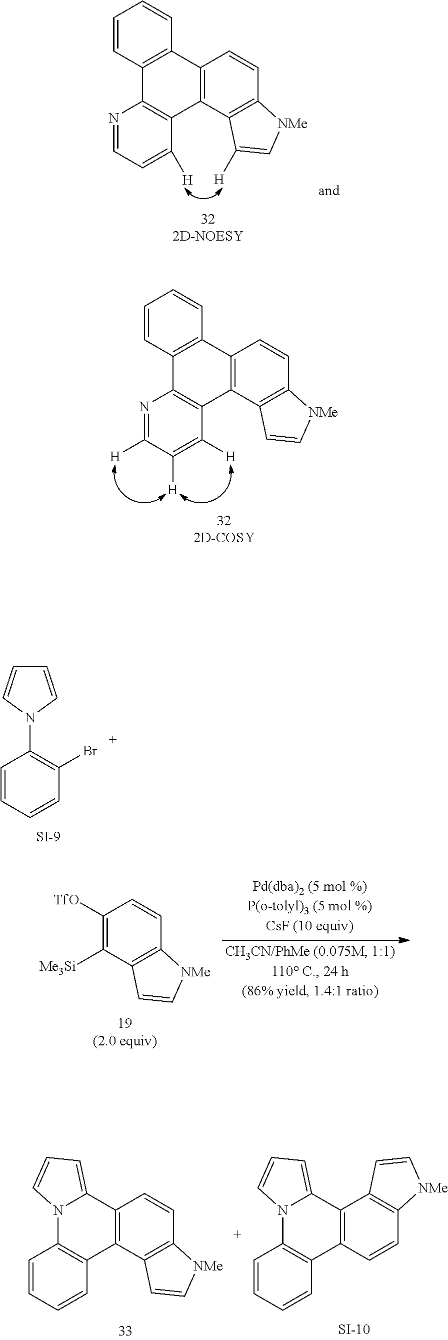

Pyrroles 33 and SI-10. Purification by flash chromatography (20:1 Hexanes:Benzene) afforded a mixture of pyrroles 33 and SI-10 (86% yield, 1.4:1 ratio, average of two experiments) as a yellow solid, mp: 135-140° C. Pyrroles 33 and SI-10: R_{f} 0.56 (4:1 Hexanes:EtOAc); ¹H-NMR (500 MHz, C₆D₆, combined): δ 9.01 (dd, J=8.0, 1.5, 1H), 8.33-8.28 (m, 1H), 8.13 (d, J=8.9, 1H), 8.01 (d, J=8.7, 1H), 7.65 (dd, J=3.0, 1.4, 1H), 7.58 (dd, J=3.0, 1.4, 1H), 7.4 (m, 3H), 7.28 (dd, J=3.2, 0.7, 1H), 7.27-7.23 (m, 2H), 7.22-7.19 (m, 1H), 7.19-7.17 (m, 2H), 7.10 (t, J=0.9, 1H), 7.09 (m, 2H), 6.92 (dd, J=4.0, 2.9, 1H), 6.84 (dd, J=3.9, 2.8, 1H), 6.67 (d, J=3.0, 1H), 6.62 (d, J=3.3, 1H), 2.96 (s, 3H), 2.92 (s, 3H); ¹³C-NMR (125 MHz, C₆D₆, combined): δ 136.7, 136.2, 134.0, 132.9, 131.5, 129.6, 128.9, 128.8, 127.60, 127.58, 127.0, 124.3, 124.2, 124.0, 123.7, 123.5, 123.4, 122.9, 121.5, 120.4, 118.9, 118.6, 117.9, 117.0, 115.3, 115.2, 113.0, 112.9, 112.7, 112.2, 111.5, 108.8, 105.6, 102.94, 102.88, 101.0, 32.22, 32.19; IR (film): 3102, 2923, 1500, 1441, 1355 cm⁻¹; DART-HR MS (m/z) [M+H⁺] calcd for C₁₉H₁₅N₂⁺, 271.12297; found 271.12191.

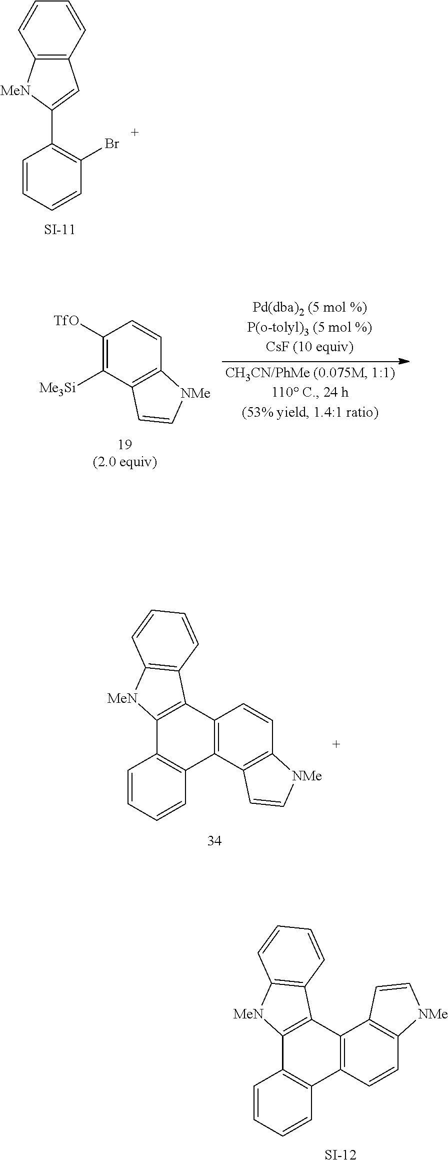

Indoles 34 and SI-12. Purification by flash chromatography (100% Hexanes→200:1 Hexanes:EtOAc→100:1 Hexanes:EtOAc) afforded an inseparable mixture of indoles 34 and SI-12 (53% yield, 1.4:1 ratio, average of two experiments) as a yellow amorphous solid. Indole 34: R_{f} 0.40 (3:1 hexanes:EtOAc); ¹H-NMR (500 MHz, C₆D₆): δ 9.74 (d, J=8.5, 1H), 9.09 (d, J=8.8, 1H), 8.92-8.88 (m, 1H), 8.48 (dd, J=8.3, 0.9, 1H) 7.70-7.65 (m, 2H), 7.59-7.43 (m, 4H), 7.29 (dd, J=7.5, 1.5, 1H), 6.81 (d, J=3.1, 1H), 3.57 (s, 3H), 3.11 (s, 3H); ¹³C-NMR (125 MHz, C₆D₆): 141.8, 134.8, 134.2, 132.7, 128.4, 128.2, 125.7, 125.3, 125.2, 124.4, 123.9, 123.8, 123.5, 123.3, 122.6, 122.1, 120.3, 118.8, 115.7, 111.4, 109.9, 103.9, 34.0, 32.4. Indole SI-12: R_{f} 0.40 (3:1 hexanes:EtOAc); ¹H-NMR (500 MHz, C₆D₆): δ 9.37 (d, J=8.1, 1H), 8.92-8.88 (m, 1H), 8.68 (d, J=9.1, 1H), 8.38 (dd, 8.4, 0.9, 1H), 7.95 (d, J=3.1, 1H), 7.59-7.43 (m, 3H), 7.40 (ddd, J=7.9, 7.1, 1.1, 1H), 7.34 (dd, J=9.1, 0.6, 1H), 7.26 (d, J=8.1, 1H), 6.72 (d, J=3.1, 1H), 3.54 (s, 3H), 3.1 (s, 3H); ¹³C-NMR (125 MHz, C₆D₆, 23 of 24 signals observed): 141.5, 136.4, 136.2, 132.8, 126.3, 125.8, 125.1, 124.8, 124.54, 124.51, 124.46, 123.7, 123.6, 123.1, 121.7, 119.2, 118.3, 115.3, 109.7, 108.1, 105.8, 34.0, 32.4; IR (film, entire mixture): 3055, 2923, 2854, 1737, 1509, 1472, 1374, 1342, 1245, 1102 cm⁻¹; HRMS-APCI (m/z) [M+H]⁺ calcd for C₂₄H₁₉N₂⁺, 335.15428; found 335.15396.

The structure of 34 was verified by 2D-NOESY of the mixture, as the following interaction was observed:

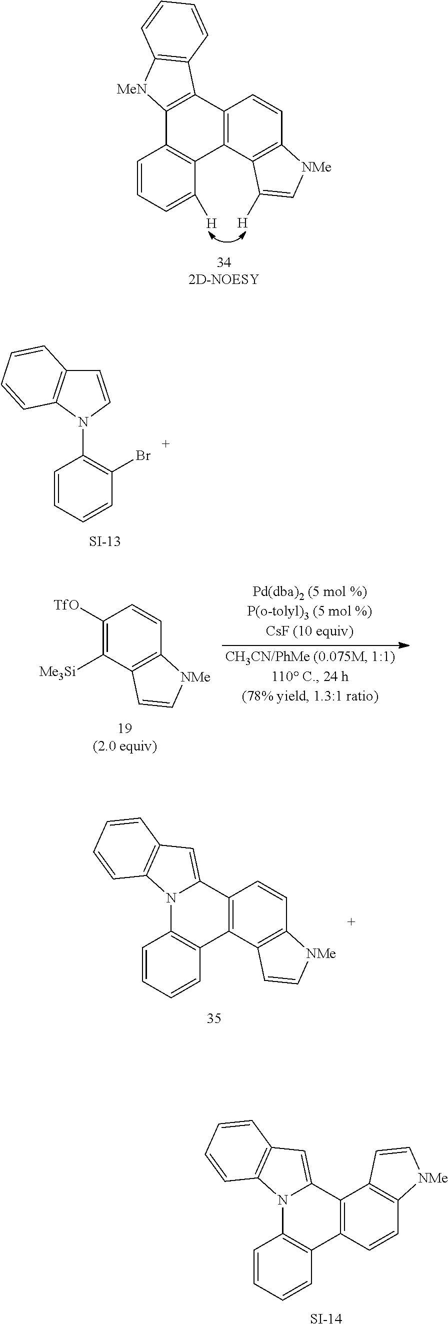

Indoles 35 and SI-14. Purification by flash chromatography (100% Hexanes→100:1 Hexanes:EtOAc→25:1 Hexanes:EtOAc→9:1 Hexanes: EtOAc, followed by a second run of 9:1 Hexanes:EtOAc) afforded an inseparable mixture of indoles 35 and SI-14 (78% yield, 1.3:1 ratio) as a bright yellow solid. Indoles 35 and SI-14: R_{f} 0.32 (4:1 hexanes:EtOAc); ¹H-NMR (500 MHz, CDCl₃, major): δ 8.43 (dd, J=8.2, 1.0, 1H), 8.28 (dd, J=8.0, 1.1, 2H), 8.04 (d, J=9.0, 1H), 7.98 (d, J=8.6, 1H), 7.79 (s, 1H), 7.41 (ddd, J=7.8, 6.8, 0.9, 1H), 7.36-7.31 (m, 1H), 7.30-7.17 (m, 3H), 7.08 (dd, J=8.9, 0.6, 1H), 6.63 (d, J=3.2, 1H), 2.94 (s, 3H); ¹H-NMR (500 MHz, CDCl₃, minor): δ 8.95 (dd, J=8.0, 1.6, 1H), 8.41 (dd, J=8.0, 1H), 8.23 (d, J=8.4, 1H), 7.98 (d, J=7.6, 1H), 7.91 (d, J=7.9, 1H), 7.41 (ddd, J=7.5, 6.9, 0.9, 1H), 7.36-7.31 (m, 1H), 7.30-7.17 (m, 4H), 7.04 (dd, J=8.7, 0.7, 1H), 6.58 (d, J=3.2, 1H), 2.92 (s, 3H); ¹³C-NMR (125 MHz, C₆D₆, combined): (45 of 46 signals observed) δ 137.6, 137.2, 136.7, 136.5, 135.6, 135.5, 134.1, 133.9, 131.7, 131.6, 129.3, 129.2, 127.6, 127.3, 124.6, 124.3, 124.0, 123.8, 123.7, 123.04, 122.95, 122.3, 122.11, 122.06, 121.6, 121.5, 121.4, 121.05, 120.98, 120.7, 119.9, 118.7, 116.8, 116.7, 116.6, 114.8, 111.3, 110.5, 103.2, 102.9, 99.8, 95.2, 32.2 (2C); IR (film): 3040, 2923, 1738, 1601, 1550, 1509, 1490, 1447, 1419, 1355; HRMS-APCI (m/z) [M+H]⁺ calcd for C₂₃H₁₇N₂⁺, 321.13862; found 321.13951.

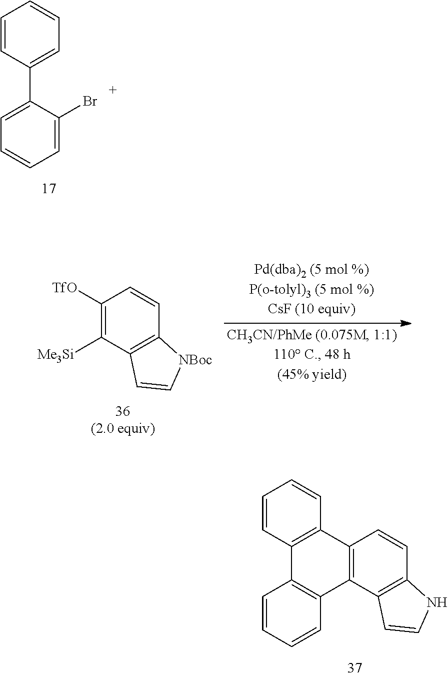 [SMILES: Brc1ccccc1-c1ccccc1 ; CC(C)(C)OC(=O)n1ccc2c([Si](C)(C)C)c(OS(=O)(=O)C(F)(F)F)ccc21 ; c1ccc2c(c1)c1ccccc1c1c3cc[nH]c3ccc21]

Indole 37. Purification by flash chromatography (100% Hexanes→200:1 Hexanes:EtOAc→10:1 Hexanes:EtOAc) afforded indole 37 (45% yield) as an orange solid. Indole 37: R_{f} 0.29 (4:1 hexanes:EtOAc); ¹H-NMR (500 MHz, CDCl₃): δ 9.35 (d, J=8.3, 1H), 8.63 (d, J=8.3, 1H), 8.59 (d, J=8.3, 1H), 8.55 (d, J=8.2, 1H), 8.48 (d, J=8.9, 1H), 7.59-7.42 (m, 5H), 7.26 (d, J=8.9, 1H), 7.45 (br s, 1H), 6.73 (t, J=2.9, 1H).

B. Piperidyne Annulation

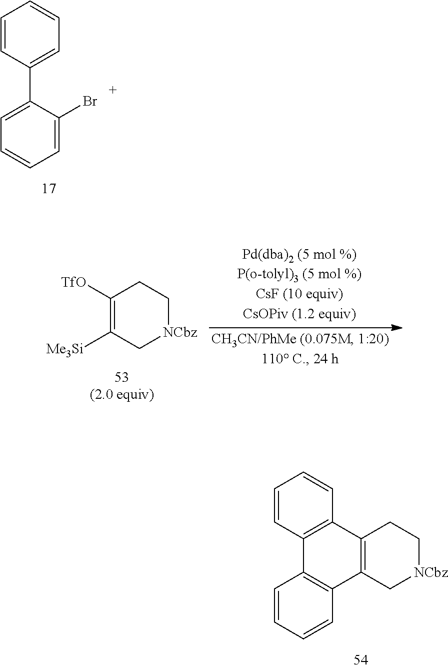 [SMILES: Brc1ccccc1-c1ccccc1 ; C[Si](C)(C)C1=C(OS(=O)(=O)C(F)(F)F)CCN(C(=O)OCc2ccccc2)C1 ; O=C(OCc1ccccc1)N1CCc2c(c3ccccc3c3ccccc23)C1]

Piperidine 54. A flame-dried 20 mL scintillation vial was charged with Pd(dba)₂ (11.3 mg, 0.064 mmol, 5 mol %). Next, toluene (4.9 mL), P(o-tolyl)₃ (6.2 mg, 0.020 mmol, 5 mol %), 2-bromobiphenyl (17) (90.6 mg, 0.389 mmol, 1.0 equiv), silyl triflate 53 (331 mg, 0.757 mmol, 2.0 equiv), CsOPiv (109 mg, 0.466 mmol, 1.2 equiv), and acetonitrile (0.24 mL) were added followed by an oven-dried magnetic stirbar and then CsF (195 mg, 1.29 mmol, 10.0 equiv). The vial was sealed with a Teflon-lined screw cap and stirred at 110° C. for 24 h. After allowing to cool to room temperature, the mixture was transferred with CH₂Cl₂ (10 mL) and H₂O (2 mL) to a 150 mL separatory funnel containing brine (15 mL). The layers were separated and the aqueous layer was extracted with CH₂Cl₂ (3×15 mL). The combined organic layers were dried over MgSO₄, filtered, and concentrated in vacuo. The resulting crude oil was purified via flash chromatography (10% Triethylamine in Hexanes→10% Triethylamine in 4:1 Hexanes:EtOAc) using silica gel neutralized with triethylamine to afford piperidine 54 (69.3 mg, 49% yield) as a yellow solid. Piperidine SI-19: R_{f} 0.39 (4:1 Hexanes:EtOAc); ¹H NMR (500 MHz, CDCl₃): δ 8.72-8.68 (m, 2H), 8.04-7.84 (m, 2H), 7.67-7.61 (m, 4H), 7.47-7.32 (m, 5H), 5.27 (s, 2H), 5.10 (s, 2H), 3.95 (s, 2H), 3.23 (d, J=16.4, 2H); IR (film): 3031, 2927, 2854, 1698, 1431, 1243, 1115; HRMS-APCI (m/z) [M+H]⁺ calcd for C₂₅H₂₂NO₂⁺, 368.16451; found 368.16218.

C. Annulations onto Ru(bpy)₃ Complexes

### Representative Procedure:

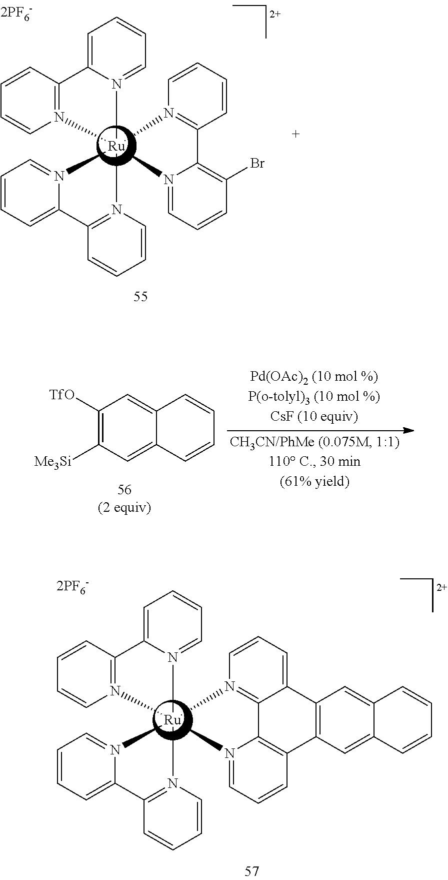

A 1-dram vial was charged with Pd(OAc)₂ (1.5 mg, 0.0067 mmol, 10 mol %). Next, bromo-Ru(bpy)₃[PF₆]2 55 (61.3 mg, 0.065 mmol, 1.0 equiv), P(o-tolyl)₃ (202 mg, 0.006 mmol, 10 mol %), acetonitrile (0.45 mL), toluene (0.45 mL), and silyl triflate 56 (40.1 mg, 0.134 mmol, 2.0 equiv) were added followed by an oven-dried magnetic stirbar and then CsF (101 mg, 1.29 mmol, 10.0 equiv). The vial was sealed with a Teflon-lined screw cap and stirred at 110° C. for 30 min. After allowing to cool to 23° C., the mixture was filtered through celite with acetonitrile (5 mL), and the resulting crude product was purified by flash chromatography (14:2:1 CH₃CN: H₂O: saturated aqueous KNO₃). KPF₆ (20 mL) was then added to the concentrated eluent to crash out the desired product. The mixture was then transferred to a 100 mL separatory funnel with CH₂Cl₂ (20 mL), and the layers were separated. The aqueous layer was extracted with CH₂Cl₂ (2×20 mL) and the combined organic layers were dried over magnesium sulfate, concentrated under reduced pressure, and redissolved in CH₃CN (5 mL). The solution was then agitated with decolorizing carbon (150 mg) and filtered, concentrated under reduced pressure, and dried on high vacuum overnight to afford the desired product in 61% yield. Ruthenium Complex 57: R_{f} 0.57 (7:2:1 CH₃CN: H₂O: saturated aqueous KNO₃); ¹H-NMR (500 MHz, CD₃CN): δ 9.41 (s, 2H), 9.29 (dd, J=8.0, 1.2, 2H), 8.56-8.51 (m, 4H), 8.30-8.27 (m, 2H), 8.11 (td, J=10, 1.4, 2H), 8.03-7.99 (m, 4H), 7.85 (dd, J=5.5, 0.5, 2H), 7.79-7.76 (m, 4H), 7.72 (dd, J=5.6, 0.5, 2H), 7.46 (ddd, J=7.8, 5.7, 1.3, 2H), 7.27 (ddd, J=7.8, 5.7, 1.3, 2H); ¹³C-NMR (500 MHz, CD₃CN): δ 158.1, 157.9, 152.9, 152.8, 152.3, 149.6, 138.8, 138.7, 134.0, 133.0, 132.3, 129.3, 129.1, 128.5, 128.4, 127.7, 126.4, 125.6, 125.2, 125.1 cm⁻¹; HR-ESI-MS (m/z) calcd for C₄₀H₂₇N₆PF₆Ru⁺, 839.1061; found 839.1050.

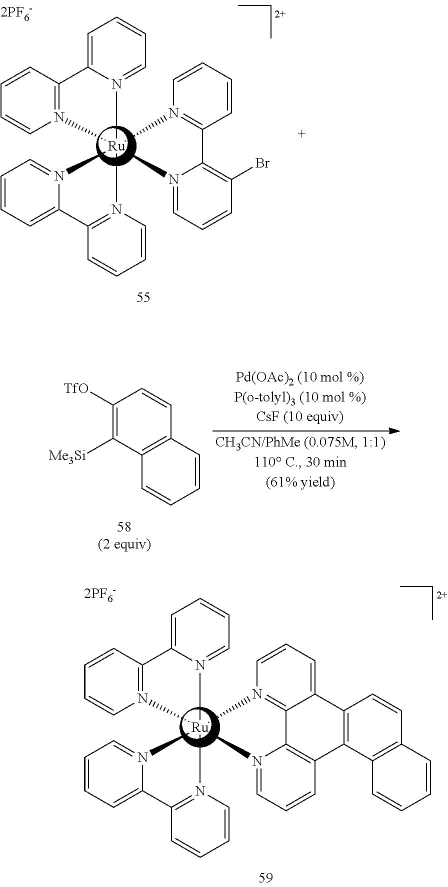

Ruthenium Complex 59. Purification by flash chromatography (14:2:1 CH₃CN: H₂O: saturated aqueous KNO₃) afforded 59 (61% yield, average of two experiments) as a red solid. Ru Complex 59: R_{f} 0.70 (7:2:1 CH₃CN: H₂O: saturated aqueous KNO₃); ¹H-NMR (600 MHz, CD₃CN): δ 9.52 (d, J=8.7, 1H), 9.29 (d, J=8.7, 1H), 8.96 (d, J=8.3, 1H), 8.82 (d, J=8.7, 1H), 8.57-8.50 (m, 4H), 8.37 (d, J=8.8, 1H), 8.24 (d, J=7.9, 1H), 8.15-8.09 (m, 4H) 8.00 (t, J=7.8, 2H), 7.89-7.78 (m, 6H), 7.69 (d, J=5.2, 1H), 7.64 (d, J=5.6, 1H), 7.49-7.44 (m, 2H), 7.23-7.20 (m, 2H); HR-ESI-MS (m/z) calcd for C₄₀H₂₇N₆PF₆Ru⁺, 839.1061; found 839.1119.

Although specific combinations of method steps and specific synthetic platforms are described above, it will be understood that modifications to the methods and synthetic platforms may be made in accordance with embodiments of the invention.

### DOCTRINE OF EQUIVALENTS

Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

What is claimed is:

1. A method for forming polycyclic aromatic hydrocarbons comprising:
providing a first cyclic alkyne, generated in situ from a first corresponding silyl triflate;
providing an oxadiazinone of formula: 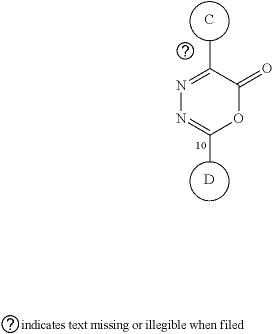
wherein rings C and D are functionalities individually chosen from: substituted or unsubstituted aromatic or heteroaromatic hydrocarbons, including polycyclic hydrocarbons;
providing a second cyclic alkyne or aryne, generated in situ from a second corresponding silyl triflate; and
reacting the first cyclic alkyne, the oxadiazinone, and the second cyclic alkyne or aryne in a plurality of sequential Diels-Alder reactions under reaction conditions to produce a polycyclic aromatic hydrocarbon comprising a 9,10-diarylanthracene scaffold.

2. The method of claim 1, wherein the plurality of sequential Diels-Alder reactions comprises a first Diels-Alder reaction, between the first cyclic alkyne and the oxadiazinone, to yield an intermediate pyrone; and a second Diels-Alder reaction, between the intermediate pyrone and the second cyclic alkyne or aryne, to yield the polycyclic aromatic hydrocarbon comprising a 9,10-diarylanthracene scaffold.

3. The method of claim 1, wherein the first cyclic alkyne comprises in its ring at least one substituted or unsubstituted heteroatom selected from: N, O, S, Se, Si, B, P; and further comprises any number of substitutions and functional groups, each individually selected from: H, halide, alkyl, aryl, heteroaryl, alkoxy, PEG.

4. The method of claim 3, wherein the first cyclic alkyne is 3,4,-piperidyne comprising an N-substitution selected from: H, alkyl, including Me, aryl, including phenyl, benzyl, carbamates, including Cbz and Boc, N-oxide, N-Borane.

5. The method of claim 1, wherein the rings C and D, independently, comprise one or more functionality selected from: an electron-donating functional group, including para-methoxyphenyl, an electron-withdrawing functional group, including para-NO₂, and a halogen atom, including F, Cl, Br, and I, heterocycles, including thiophene, alkenes, alkynes.

6. The method of claim 5, wherein one or both of the rings C and D comprise a functional handle and wherein the functional handle is used to further extend, including polymerize, the polycyclic aromatic hydrocarbon comprising a 9,10-diarylanthracene scaffold and at least one heteroatom.

7. The method of claim 1, wherein the second cyclic alkyne or aryne comprises at least one feature selected from:
comprises at least one substituted or unsubstituted heteroatom selected from: N, O, S. Se, Si, B, P;
is polycyclic or polyheterocyclic, wherein the cycles are aromatic, non-aromatic, or both;
comprises any number of substitutions or functional groups, each individually selected from: H, alkyl, aryl, heteroaryl, electron-withdrawing groups, electron-donating groups.

8. The method of claim 7, wherein the second cyclic alkyne or aryne is selected from: benzyne, naphthalyne, indolyne, and cyclohexyne, including cyclohexyne with at least one heteroatom, wherein the at least one heteroatom may be further functionalized.

9. The method of claim 1, wherein the reaction conditions comprise additional reagents, reagent stoichiometry, and physical conditions selected to promote an elimination of silyl triflate from the first and the second corresponding silyl triflates, and to promote Diels-Alder reactions between the first cyclic alkyne and the oxadiazinone, and between an intermediate pyrone and the second cyclic alkyne or aryne.

10. The method of claim 1 wherein the reaction conditions comprise an additional reagent providing F⁻, a solvent, a temperature, and a period of time.

11. The method of claim 10, wherein the additional reagent providing F⁻ is selected from: CsF, LiF, KF, NaF, N(nBu)₄F, HF, HF.pyridine, Poly[4-vinylpyridinium poly(hydrogen fluoride)], tetrabutylammonium difluorotriphenylsilicate.

12. The method of claim 10, wherein the solvent is selected from: acetonitrile, toluene, tetrahydrofuran, chloroform, dichloromethane, any other ethereal and halogenated solvents, and any mixture thereof.

13. The method of claim 10, wherein reacting the first cyclic alkyne, the oxadiazinone, and the second cyclic alkyne or aryne is conducted in a stepwise manner, wherein
first, 1 equivalent of the first cyclic alkyne is reacted with 1 to 5 equivalents of the oxadiazinone and 1 to 10 equivalents of CsF in acetonitrile as 0.1 M solution relative to the first cyclic alkyne for 12 to 24 hours to produce an intermediate pyrone; and
next, 1 equivalent of the intermediate pyrone is reacted with 1 to 5 equivalents of the second cyclic alkyne or aryne and 1 to 10 equivalents of CsF in acetonitrile as 0.1 M solution relative to the intermediate pyrone for 12 to 24 hours.

14. The method of claim 10, wherein
first, 1 equivalent of the first cyclic alkyne is reacted with 2 equivalents of the oxadiazinone and 2 equivalents of CsF in acetonitrile as 0.1 M solution relative to the first cyclic alkyne for 14 to 18 hours to produce an intermediate pyrone; and
next, 1 equivalent of the intermediate pyrone is reacted with 2 equivalents of the second cyclic alkyne or aryne and 5 equivalents of CsF in acetonitrile as 0.1 M solution relative to the intermediate pyrone for 18 hours.

15. The method of claim 13, wherein the intermediate pyrone is isolated and purified prior to being reacted with the second cyclic alkyne or aryne.

16. The method of claim 10, wherein the reacting of 1 equivalent of the first cyclic alkyne, 1 to 5 equivalents of the oxadiazinone, and 1 to 5 equivalents of the second cyclic alkyne or aryne is conducted in a one-pot manner, with addition of 1 to 10 equivalents of CsF in acetonitrile as 0.1 M solution relative to the first cyclic or heterocyclic alkyne for 12 to 24 hours.

17. The method of claim 16, wherein the reacting of 1 equivalent of the first cyclic alkyne, 1 equivalent of the oxadiazinone, and 1 equivalent of the second cyclic alkyne or aryne is conducted in a one-pot manner, with addition of 3 equivalents of CsF in acetonitrile as 0.1 M solution relative to the first cyclic alkyne for 14 hours.

18. The method of claim 1, wherein the polycyclic aromatic hydrocarbon comprising a 9,10-diarylanthracene scaffold further comprises at least one heteroatom.

19. The method of claim 18, wherein at least one heteroatom is nitrogen.

20. A heteroatom-containing polycyclic aromatic hydrocarbon selected from the group consisting of:

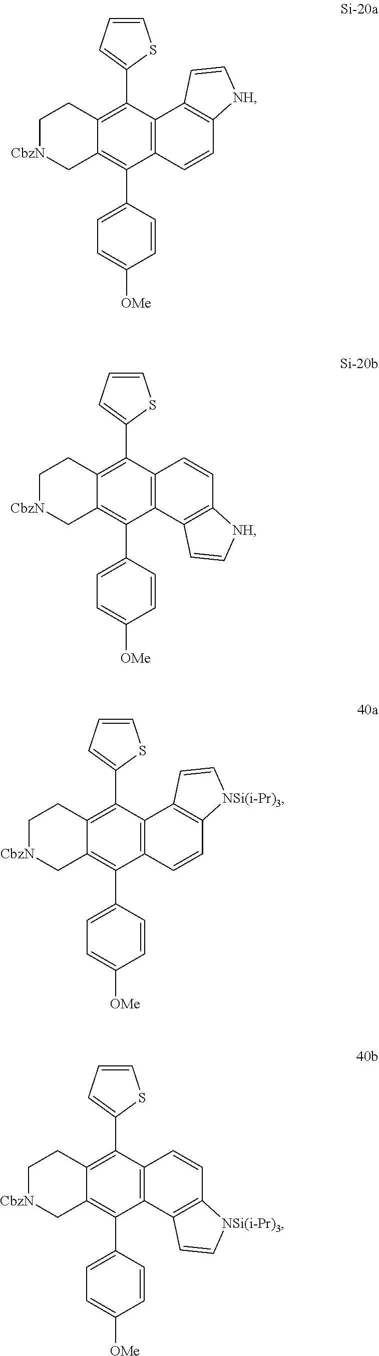

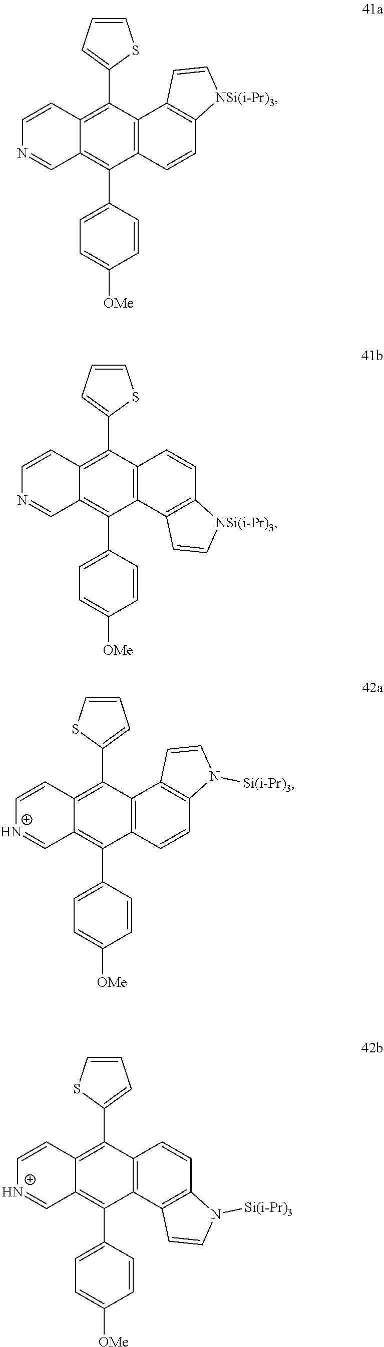

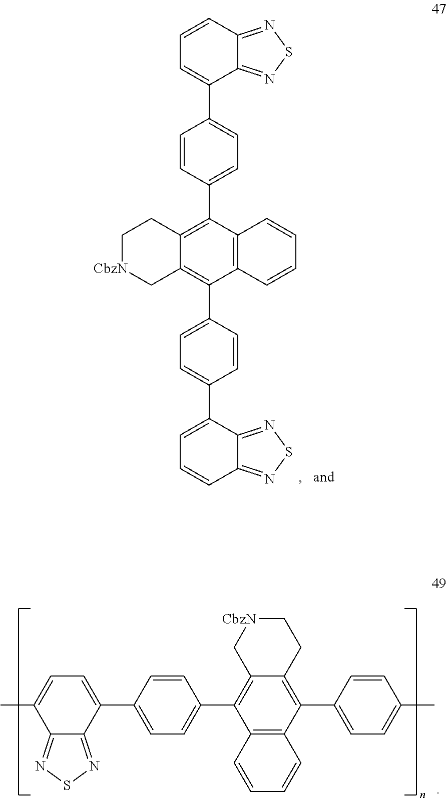

21. A method for forming polycyclic aromatic hydrocarbons comprising:
providing a cyclic alkyne or heterocyclic aryne, generated in situ from a corresponding silyl triflate;
providing a halo-biaryl; and
reacting the cyclic alkyne or aryne and the halo-biaryl in a transition metal-catalyzed cross-coupling reaction under reaction conditions to produce a polycyclic aromatic hydrocarbon comprising a triphenylene scaffold.

22. The method of claim 21, wherein the cyclic alkyne or aryne comprises at least one feature selected from:
comprises at least one substituted or unsubstituted heteroatom selected from: N, O, S, Se, Si, B, P;
is polycyclic or polyheterocyclic, wherein the cycles are aromatic, non-aromatic, or both;
comprises any number of substitutions or functional groups, each individually selected from: H, alkyl, aryl, heteroaryl, electron-withdrawing groups, electron-donating groups.

23. The method of claim 22, wherein the cyclic alkyne or aryne is selected from: naphthalyne, indolyne, carbazolyne, and cyclohexyne, including cyclohexyne with at least one heteroatom, wherein the at least one heteroatom may be further functionalized.

24. The method of claim 21, wherein the halo-biaryl is of formula: 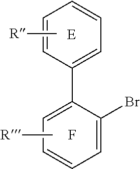
wherein rings E and F are functionalities individually chosen from: substituted or unsubstituted aromatic or heteroaromatic hydrocarbons, including polycyclic hydrocarbons; and
wherein R″ and R″′ are further functionalities individually chosen from H, alkyl, alkoxy, NO₂, amine, alkyl amine.

25. The method of claim 24, wherein the halo-biaryl is selected from:

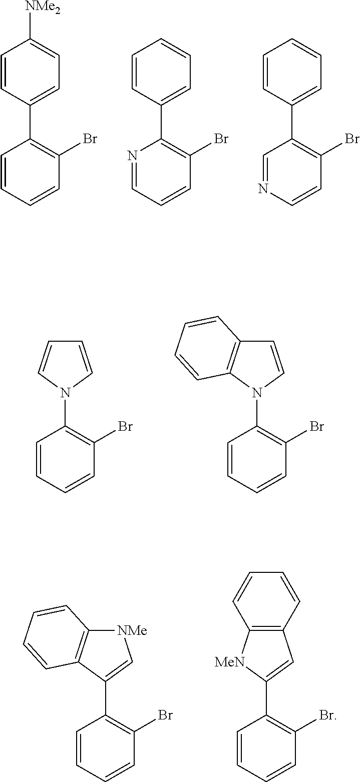

26. The method of claim 21, wherein the reaction conditions comprise additional reagents, reagent stoichiometry, and physical conditions selected to promote an elimination of silyl triflate from the corresponding silyl triflate, and to promote the transition metal-catalyzed cross-coupling reaction between the cyclic alkyne or aryne and the halo-biaryl.

27. The method of claim 26, wherein the reaction conditions comprise an additional reagent providing F⁻, the group 10 metal catalyst, a ligand, a solvent, reflux conditions, and a period of time.

28. The method of claim 27, wherein the additional reagent providing F⁻ is selected from: CsF, LiF, KF, NaF, N(nBu)₄F, HF, HF.pyridine, Poly[4-vinylpyridinium poly(hydrogen fluoride)], tetrabutylammonium difluorotriphenylsilicate.

29. The method of claim 27, wherein the cyclic alkyne or aryne is a cyclic or heterocyclic alkyne and an amount of CsOPiv is added.

30. The method of claim 27, wherein the group 10 metal catalyst is Pd selected from: Pd(dba)₂ and Pd(OAc)₂; and the ligand including P(o-tolyl)₃.

31. The method of claim 27, wherein the reaction conditions comprise one selected from the group consisting of:
1 equivalent of the halo-biaryl, 2 equivalents of the cyclic alkyne or aryne, 1 to 20 equivalents of CsF, 5 to 100 mol % Pd⁰, 1:1 ratio of Pd⁰ to its ligand, a solvent or solvent mixture allowing heating to 90-150° C., 0.5 to 24 hours;
1 equivalent of the halo-biaryl, 2 equivalents of the cyclic alkyne or aryne, 10 equivalents of CsF, 5 mol % Pd(dba)2, 5 mol % P(o-tolyl)₃, 1:1 acetonitrile/toluene 0.075M relative to halo-biaryl solvent mixture, 110° C., 24 hours;
wherein the halo-biaryl is a part of a transition metal organometallic complex and the reaction conditions comprise: 1 equivalent of the halo-biaryl, 2 equivalents of the cyclic alkyne or aryne, 10 equivalents of CsF, 10 mol % Pd(OAc)₂, 10 mol % P(o-tolyl)₃, 1:1 acetonitrile/toluene 0.075M relative to halo-biaryl solvent mixture, 110° C., 0.5 hours; and
wherein the halo-biaryl is a part of a transition metal organometallic complex and the reaction conditions comprise: 1 equivalent of the halo-biaryl, 2 equivalents of the cyclic alkyne or aryne, 10 equivalents of CsF, 10 mol % Pd(OAc)2, 10 mol % P(o-tolyl)₃, 1:1 acetonitrile/toluene 0.075M relative to halo-biaryl solvent mixture, 110° C., 0.5 hours and wherein the transition metal organometallic complex comprises a transition metal selected from: Co, Ir, Rh, Ni, Pd, Pt, Zn, Cu, Fe, Mn, Os.

32. The method of claim 21, wherein at least one heteroatom is employed to further decorate or otherwise extend the polycyclic aromatic hydrocarbon comprising a triphenylene scaffold and at least one heteroatom.

33. The method of claim 21, wherein the halo-biaryl is bromo-biaryl.

34. A heteroatom-containing polycyclic aromatic hydrocarbon selected from the group consisting of: 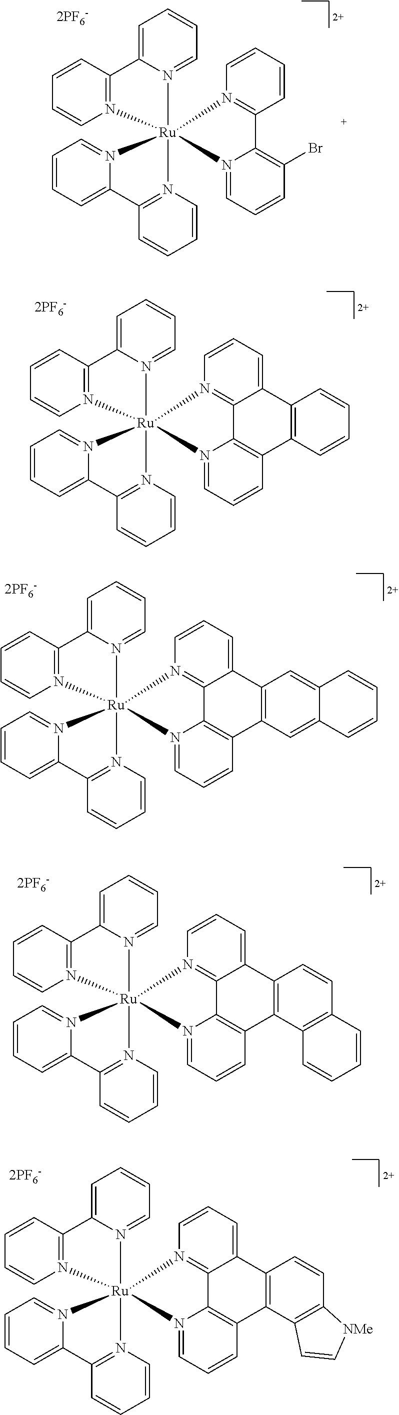 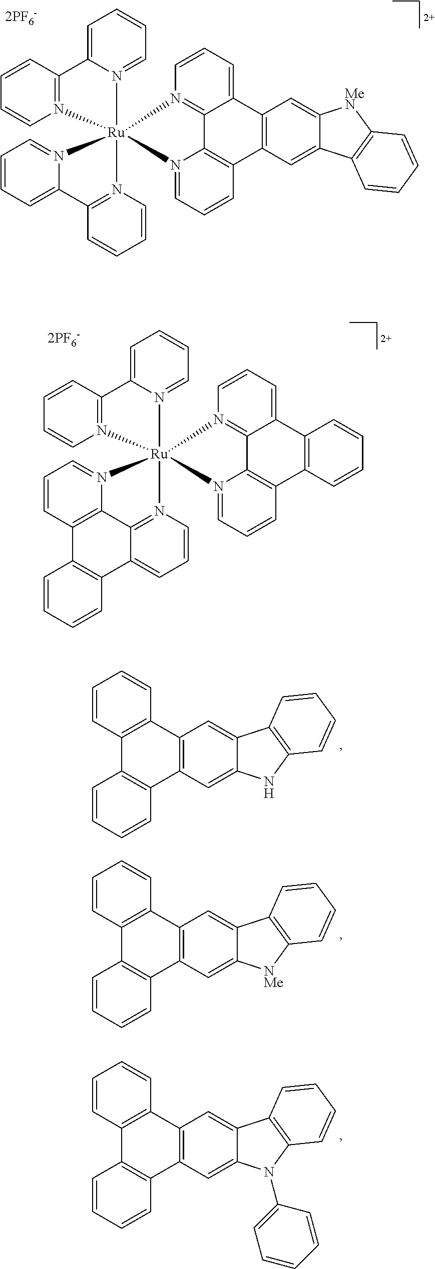 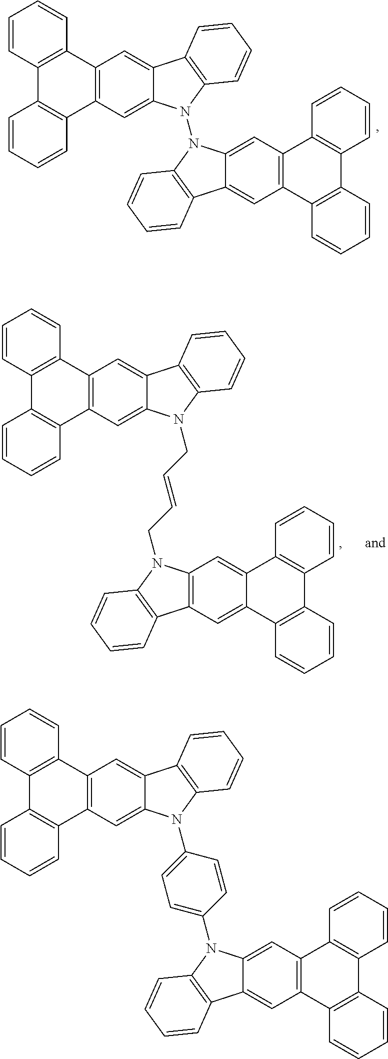 [SMILES: c1ccc2c(c1)c1ccccc1c1cc3c(cc21)c1ccccc1n3-n1c2ccccc2c2cc3c4ccccc4c4ccccc4c3cc21 ; C(=C\Cn1c2ccccc2c2cc3c4ccccc4c4ccccc4c3cc21)/Cn1c2ccccc2c2cc3c4ccccc4c4ccccc4c3cc21 ; c1ccc2c(c1)c1ccccc1c1cc3c(cc21)c1ccccc1n3-c1ccc(-n2c3ccccc3c3cc4c5ccccc5c5ccccc5c4cc32)cc1]
